(19) United States
(12) Patent Application Publication
(10) Pub. No.: US 2020/0061026 A1
(43) Pub. Date: Feb. 27, 2020

(54) METHODS AND COMPOSITIONS FOR PREVENTING VECTOR-BORNE DISEASE TRANSMISSION

(71) Applicants: The Scripps Research Institute, La Jolla, CA (US); TropIQ Health Sciences, Nijmegen (NL)

(72) Inventors: Matthew S. TREMBLAY, San Diego, CA (US); Arnab K. CHATTERJEE, San Diego, CA (US); Peter G. SCHULTZ, La Jolla, CA (US); Koen DECHERING, Nijmegen (NL); Marie MIGLIANICO, Nijmegen (NL)

(21) Appl. No.: 16/346,425

(22) PCT Filed: Oct. 30, 2017
(86) PCT No.: PCT/US17/59084

§ 371 Date: Apr. 30, 2019

Related U.S. Application Data
(60) Provisional application No. 62/415,287, filed on Oct. 31, 2016.

(51) Int. Cl. A61K 31/42; A61K 9/00; A61K 31/397; A61P 33/14

(52) U.S. Cl. CPC A61K 31/42; A61P 33/14; A61K 31/397; A61K 9/0053

(57) ABSTRACT

Disclosed herein are methods of preventing transmission of vector-borne diseases by mass administration of insecticidal drugs to a human population. Exemplary vectors targeted by the drugs are of the class Insecta, and include the genera Anopheles and Aedes.

### CROSS-REFERENCE

This application claims benefit to U.S. Provisional Application No. 62/415,287 filed Oct. 31, 2016, the entirety of which is incorporated by reference herein.

### BACKGROUND OF THE INVENTION

Vectors are living organisms that can transmit infectious diseases between humans and between humans and animals. Exemplary vectors include insects such as mosquitos, triatomine bugs, tsetse flies, and black flies, as well as ectoparasites such as ticks and fleas. Generally, the infectious diseases are caused by organisms transferred between the vector and human or animal. Organisms may be transferred when a vector ingests the organism during a bite or blood meal with an infected human or animal, and then injects the organism into a new human or animal during a subsequent bite or blood meal. Exemplary organisms which are causative agents of disease include parasites, such as those of the Plasmodium genus that cause malaria; and viruses, such as Zika virus.

### SUMMARY OF THE INVENTION

The present disclosure provides methods and compositions for treating or preventing vector-borne transmission of infectious organisms by administrating to a human or animal a compound that is lethal to the vector. During a bite or blood meal with the human or animal, the vector ingests the compound and subsequently dies, thus preventing the vector from further transmitting the organism to another host. In particular methods, a compound is formulated for mass drug administration to humans, whereby a significant portion of a human population at risk for acquiring a vector-borne disease is administered the compound. Formulations include those which allow for administration of the compound in a single course which may be lethal to an infectious organism for months at a time. In such cases, the single course may be administered to correspond with the beginning of a particular season, when organisms like the mosquito are prevalent and there is an increased risk of transmission.

In one aspect, provided herein is a method of vector control comprising administering an insecticide to a human; wherein the insecticide is lethal to a vector exposed to the administered insecticide during a bite or blood meal with the human. In some embodiments, the human is administered the insecticide in: (a) a single dose or (b) a plurality of doses over a course of less than or equal to about 3 days; and wherein the single dose or the plurality of doses is administered once or not more frequently than every 3 months. In some embodiments, the single dose or the plurality of doses is administered not more frequently than every 9 months. In some embodiments, if the vector is exposed to the administered insecticide within about 30, 60, 90, or 120 days after administration, the administered insecticide is effective in killing the vector. In some embodiments, the insecticide is lethal to the vector within about 8, 7, 6, 5, 4, 3, 2 or 1 days of exposure. In some embodiments, the vector is an insect vector selected from a mosquito, triatomine bug, tsetse fly, sandfly, and black fly. In some embodiments, the insect vector is a mosquito of a genus selected from Aedes, Anopheles, Culex, and Phlebotomus. In some embodiments, the insect vector is a mosquito capable of transmitting a parasite. In some embodiments, the parasite is of the Plasmodium genus. In some embodiments, the insect vector is a mosquito capable of transmitting a virus selected from a flavivirus, bunyavirus, and a togavirus. In some embodiments, the insecticide is an ectoparasiticide. In some embodiments, the insecticide is an isoxazoline compound. In some embodiments, the insecticide is a compound having Formula (I), or pharmaceutically acceptable salt or solvate thereof:

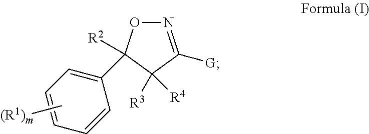

wherein:
each R¹ is independently selected from -D, —OR⁵, —SR⁵, —N(R⁶)(R⁷), —F, —Cl, —Br, —I, —C(O)R⁵, —CO₂R⁵, —CN, —NO₂, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
   each R⁵ is independently selected from —H, substituted or unsubstituted C₁-C₆alkyl, substituted or unsubstituted C₃-C₇cycloalkyl, substituted or unsubstituted aryl, and substituted or unsubstituted heteroaryl;
   each R⁶ and R⁷ are independently selected from —H, substituted or unsubstituted C₁-C₇alkyl, substituted or unsubstituted C₁-C₇fluoroalkyl, and substituted or unsubstituted C₁-C₇heteroalkyl;
   R⁶ and R⁷ can optionally be taken together with the N-atom to which they are attached to form a N-containing heterocycle;
R² is —H, —F, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, substituted or unsubstituted benzyl, or substituted or unsubstituted heteroaryl;
each R³ and R⁴ are independently selected from —H, —F, substituted or unsubstituted C₁-C₇alkyl, substituted or unsubstituted C₁-C₇fluoroalkyl, and substituted or unsubstituted C₁-C₇heteroalkyl; substituted or unsubstituted C₃-C₇cycloalkyl, substituted or unsubstituted C₂-C₆heterocycloalkyl, substituted or unsubstituted aryl, and substituted or unsubstituted heteroaryl;
m is 0, 1, 2, 3, 4, or 5; and
G is substituted or unsubstituted aryl or substituted or unsubstituted heteroaryl.

In some embodiments, G is

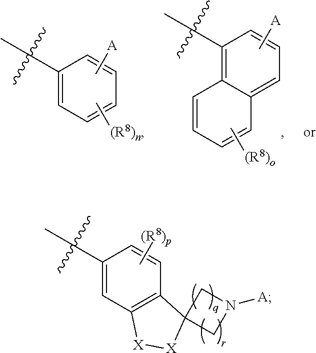

each R⁸ is independently selected from -D, —N(R⁶)(R⁷), —F, —Cl, —Br, —I, substituted or unsubstituted C₁-C₇alkyl, substituted or unsubstituted C₂-C₇alkenyl, substituted or unsubstituted C₂-C₇alkynyl, substituted or unsubstituted C₁-C₇fluoroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
two R⁸ groups can optionally be taken together with the adjacent carbon atoms to which they are attached to form aromatic or partially saturated carbocycle or heterocycle;
each X is independently selected from —O—, —S—, —S(═O)—, —S(═O)₂—, —NR⁶—, —C(═O)—, and —(CR⁹R¹⁰)ₛ—, wherein each R⁹ and R¹⁰ are independently selected from —H, -D, —F, —OR⁵, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl; s is 1, 2, or 3;
n is 0, 1, 2, 3, or 4;
o is 0, 1, 2, 3, 4, 5, or 6;
p is 0, 1, 2, or 3;
q is 0, 1, or 2;
r is 0, 1, or 2;
A is

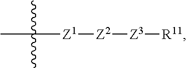

wherein
Z¹, Z², and Z³ are independently absent or selected from —(CR¹²R¹³)ᵤ—, —NR⁶—, —C(═O)—, —S(═O)—, —S(═O)₂—, —C(═O)(CR¹²R¹³)ᵤ—, —(CR¹²R¹³)ᵤC(═O)—, —C(═O)NR⁶—, —NR⁶C(═O)—, —C(═O)O—, —OC(═O)—, —OC(═O)NR⁶—, —NR⁶C(═O)O—, —NR⁶C(═O)NR⁶—, —C(═O)NR⁶(CR¹²R¹³)ᵤ—, —NR⁶C(═O)(CR¹²R¹³)ᵤ—, —(CR¹²R¹³)ᵤC(═O)NR⁶—, and —(CR¹²R¹³)ᵤNR⁶C(═O)—;
   each R¹² and R¹³ are independently selected from —H, -D, —F, —OR⁵, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl;
   u is 1, 2, 3, or 4; and
R¹¹ is substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, or substituted or unsubstituted heteroaryl.

In some embodiments,

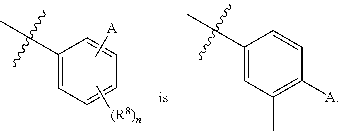

In some embodiments,

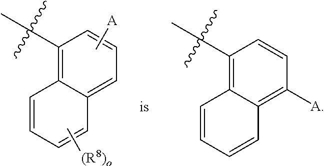

In some embodiments,

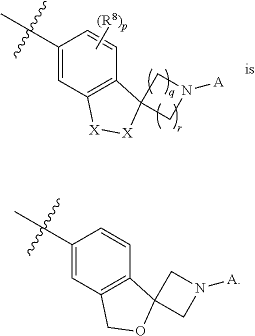

In some embodiments, A is

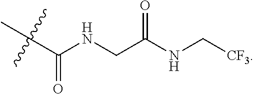 [SMILES: *C(=O)NCC(=O)NCC(F)(F)F]

In some embodiments, A is

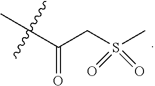 [SMILES: *C(=O)CS(C)(=O)=O]

In some embodiments, the compound of Formula (I) is fluralaner,

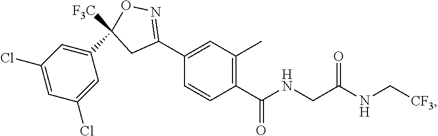 [SMILES: Cc1cc(C2=NO[C@](c3cc(Cl)cc(Cl)c3)(C(F)(F)F)C2)ccc1C(=O)NCC(=O)NCC(F)(F)F]

or pharmaceutically acceptable salt or solvate thereof.

In some embodiments, the compound of Formula (I) is (S)-fluralaner,

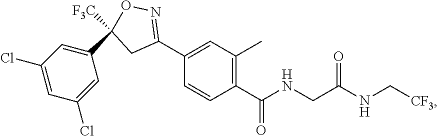 [SMILES: Cc1cc(C2=NO[C@@](c3cc(Cl)cc(Cl)c3)(C(F)(F)F)C2)ccc1C(=O)NCC(=O)NCC(F)(F)F]

or pharmaceutically acceptable salt or solvate thereof.

In some embodiments, the compound of Formula (I) is afoxolaner,

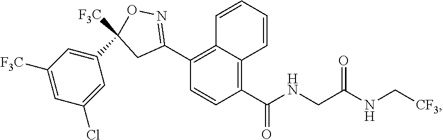 [SMILES: O=C(CNC(=O)c1ccc(C2=NO[C@](c3cc(Cl)cc(C(F)(F)F)c3)(C(F)(F)F)C2)c2ccccc12)NCC(F)(F)F]

or pharmaceutically acceptable salt or solvate thereof.

In some embodiments, the compound of Formula (I) is (S)-afoxolaner,

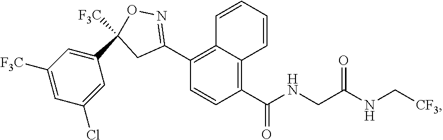 [SMILES: O=C(CNC(=O)c1ccc(C2=NO[C@@](c3cc(Cl)cc(C(F)(F)F)c3)(C(F)(F)F)C2)c2ccccc12)NCC(F)(F)F]

or pharmaceutically acceptable salt or solvate thereof.

In some embodiments, the compound of Formula (I) is (R)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide,

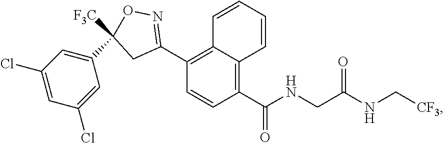 [SMILES: O=C(CNC(=O)c1ccc(C2=NO[C@](c3cc(Cl)cc(Cl)c3)(C(F)(F)F)C2)c2ccccc12)NCC(F)(F)F]

or pharmaceutically acceptable salt or solvate thereof.

In some embodiments, the compound of Formula (I) is (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide,

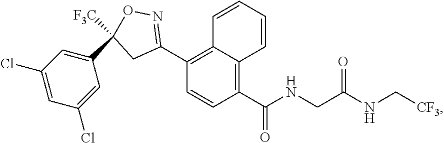 [SMILES: O=C(CNC(=O)c1ccc(C2=NO[C@@](c3cc(Cl)cc(Cl)c3)(C(F)(F)F)C2)c2ccccc12)NCC(F)(F)F]

or pharmaceutically acceptable salt or solvate thereof.

In some embodiments, the compound of Formula (I) is sarolaner,

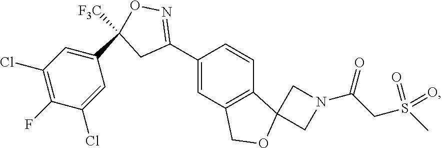 [SMILES: CS(=O)(=O)CC(=O)N1CC2(C1)OCc1cc(C3=NO[C@@](c4cc(Cl)c(F)c(Cl)c4)(C(F)(F)F)C3)ccc12]

or pharmaceutically acceptable salt or solvate thereof.

In some embodiments, the insecticide is administered in an oral dosage form. In some embodiments, each dose of the insecticide administered to the human is between about 1 mg/kg and about 50 mg/kg. In some embodiments, each dose of the insecticide administered to the human is between about 150 mg and about 750 mg.

In another aspect, provided herein is a method of preventing transmission of a disease-causing organism from a vector to a human population, the method comprising administering to each of a plurality of individuals of the population an insecticide; wherein the vector is exposed to the administered insecticide during a bite or blood meal with a member of the plurality of individuals, and if the vector is exposed to the administered insecticide within about 30, 60, 90, or 120 days after administration, the administered insecticide is effective in killing the vector. In some embodiments, the insecticide is administered to each of the plurality of individuals in a single dose, and the single dose is optionally repeated no more than every 3 months. In some embodiments, the insecticide is administered to each of the plurality of individuals in a plurality of doses over a course of less than or equal to about 3 days, and the plurality of doses is optionally repeated no more than every 3 months. In some embodiments, the vector is an insect vector selected from a mosquito, triatomine bug, tsetse fly, sandfly, and black fly. In some embodiments, the insect vector is a mosquito of a genus selected from Aedes, Anopheles, Culex, and Phlebotomus. In some embodiments, the insect vector is a mosquito capable of transmitting a parasite. In some embodiments, the parasite is of the Plasmodium genus. In some embodiments, the insect vector is a mosquito capable of transmitting a virus selected from a flavivirus, bunyavirus, and a togavirus. In some embodiments, the insecticide is administered in an oral dosage form. In some embodiments, each dose of the insecticide administered to the plurality of individuals is between about 1 mg/kg and about 50 mg/kg. In some embodiments, each dose of the insecticide administered to the plurality of individuals is between about 150 mg and about 750 mg. In some embodiments, the insecticide is an ectoparasiticide. In some embodiments, the insecticide is an isoxazoline compound. In some embodiments, the insecticide is a compound having Formula (I), or pharmaceutically acceptable salt or solvate thereof:

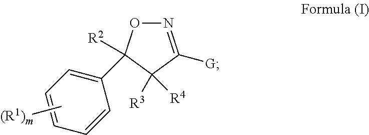

wherein:
each R¹ is independently selected from -D, —OR⁵, —SR⁵, —N(R⁶)(R⁷), —F, —Cl, —Br, —I, —C(O)R⁵, —CO₂R⁵, —CN, —NO₂, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
   each R⁵ is independently selected from —H, substituted or unsubstituted C₁-C₆alkyl, substituted or unsubstituted C₃-C₇cycloalkyl, substituted or unsubstituted aryl, and substituted or unsubstituted heteroaryl;
   each R⁶ and R⁷ are independently selected from —H, substituted or unsubstituted C₁-C₇alkyl, substituted or unsubstituted C₁-C₇fluoroalkyl, and substituted or unsubstituted C₁-C₇heteroalkyl;
   R⁶ and R⁷ can optionally be taken together with the N-atom to which they are attached to form a N-containing heterocycle;
R² is —H, —F, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, substituted or unsubstituted benzyl, or substituted or unsubstituted heteroaryl;
each R³ and R⁴ are independently selected from —H, —F, substituted or unsubstituted C₁-C₇alkyl, substituted or unsubstituted C₁-C₇fluoroalkyl, and substituted or unsubstituted C₁-C₇heteroalkyl; substituted or unsubstituted C₃-C₇cycloalkyl, substituted or unsubstituted C₂-C₆heterocycloalkyl, substituted or unsubstituted aryl, and substituted or unsubstituted heteroaryl;
m is 0, 1, 2, 3, 4, or 5; and
G is substituted or unsubstituted aryl or substituted or unsubstituted heteroaryl.

In some embodiments, G is

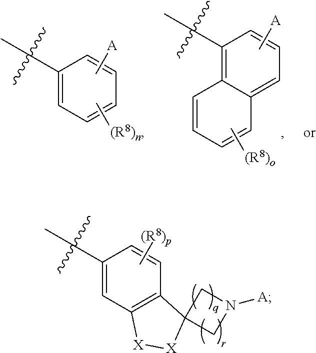

each R⁸ is independently selected from -D, —N(R⁶)(R⁷), —F, —Cl, —Br, —I, substituted or unsubstituted C₁-C₇alkyl, substituted or unsubstituted C₂-C₇alkenyl, substituted or unsubstituted C₂-C₇alkynyl, substituted or unsubstituted C₁-C₇fluoroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
two R⁸ groups can optionally be taken together with the adjacent carbon atoms to which they are attached to form aromatic or partially saturated carbocycle or heterocycle;
each X is independently selected from —O—, —S—, —S(═O)—, —S(═O)₂—, —NR⁶—, —C(═O)—, and —(CR⁹R¹⁰)ₛ—, wherein each R⁹ and R¹⁰ are independently selected from —H, -D, —F, —OR⁵, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl; s is 1, 2, or 3;
n is 0, 1, 2, 3, or 4;
o is 0, 1, 2, 3, 4, 5, or 6;
p is 0, 1, 2, or 3;
q is 0, 1, or 2;
r is 0, 1, or 2;
A is

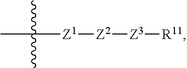

wherein
Z¹, Z², and Z³ are independently absent or selected from —(CR¹²R¹³)ᵤ—, —NR⁶—, —C(═O)—, —S(═O)—, —S(═O)₂—, —C(═O)(CR¹²R¹³)ᵤ—, —(CR¹²R¹³)ᵤC(═O)—, —C(═O)NR⁶—, —NR⁶C(═O)—, —C(═O)O—, —OC(═O)—, —OC(═O)NR⁶—, —NR⁶C(═O)O—, —NR⁶C(═O)NR⁶—, —C(═O)NR⁶(CR¹²R¹³)ᵤ—, —NR⁶C(═O)(CR¹²R¹³)ᵤ—, —(CR¹²R¹³)ᵤC(═O)NR⁶—, and —(CR¹²R¹³)ᵤNR⁶C(═O)—;
   each R¹² and R¹³ are independently selected from —H, -D, —F, —OR⁵, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl;
   u is 1, 2, 3, or 4; and
R¹¹ is substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, or substituted or unsubstituted heteroaryl.

In some embodiments,

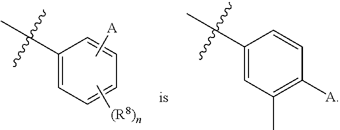

In some embodiments,

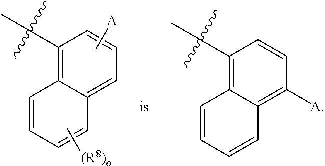

In some embodiments,

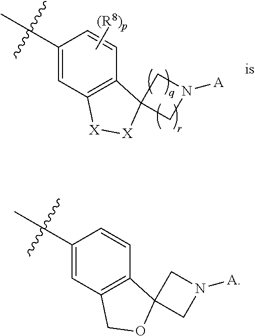

In some embodiments, A is

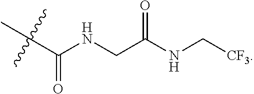 [SMILES: *C(=O)NCC(=O)NCC(F)(F)F]

In some embodiments, A is

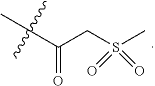 [SMILES: *C(=O)CS(C)(=O)=O]

In some embodiments, the compound of Formula (I) is fluralaner,

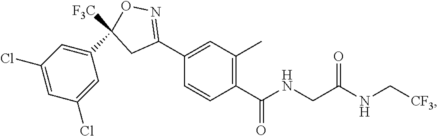 [SMILES: Cc1cc(C2=NO[C@](c3cc(Cl)cc(Cl)c3)(C(F)(F)F)C2)ccc1C(=O)NCC(=O)NCC(F)(F)F]

or pharmaceutically acceptable salt or solvate thereof.

In some embodiments, the compound of Formula (I) is (S)-fluralaner,

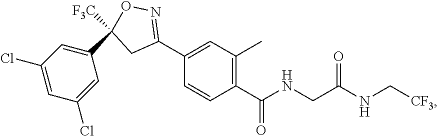 [SMILES: Cc1cc(C2=NO[C@@](c3cc(Cl)cc(Cl)c3)(C(F)(F)F)C2)ccc1C(=O)NCC(=O)NCC(F)(F)F]

or pharmaceutically acceptable salt or solvate thereof.

In some embodiments, the compound of Formula (I) is afoxolaner,

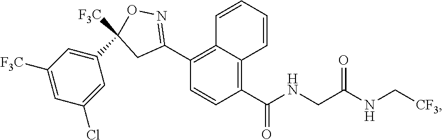 [SMILES: O=C(CNC(=O)c1ccc(C2=NO[C@](c3cc(Cl)cc(C(F)(F)F)c3)(C(F)(F)F)C2)c2ccccc12)NCC(F)(F)F]

or pharmaceutically acceptable salt or solvate thereof.

In some embodiments, the compound of Formula (I) is (S)-afoxolaner,

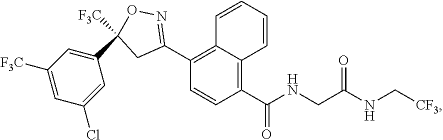 [SMILES: O=C(CNC(=O)c1ccc(C2=NO[C@@](c3cc(Cl)cc(C(F)(F)F)c3)(C(F)(F)F)C2)c2ccccc12)NCC(F)(F)F]

or pharmaceutically acceptable salt or solvate thereof.

In some embodiments, the compound of Formula (I) is (R)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide,

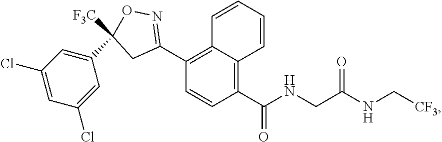 [SMILES: O=C(CNC(=O)c1ccc(C2=NO[C@](c3cc(Cl)cc(Cl)c3)(C(F)(F)F)C2)c2ccccc12)NCC(F)(F)F]

or pharmaceutically acceptable salt or solvate thereof.

In some embodiments, the compound of Formula (I) is (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide,

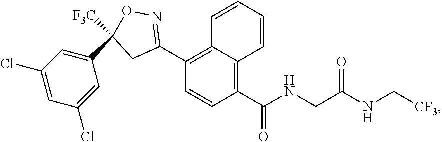 [SMILES: O=C(CNC(=O)c1ccc(C2=NO[C@@](c3cc(Cl)cc(Cl)c3)(C(F)(F)F)C2)c2ccccc12)NCC(F)(F)F]

or pharmaceutically acceptable salt or solvate thereof.

In some embodiments, the compound of Formula (I) is sarolaner,

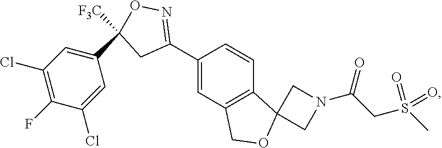 [SMILES: CS(=O)(=O)CC(=O)N1CC2(C1)OCc1cc(C3=NO[C@@](c4cc(Cl)c(F)c(Cl)c4)(C(F)(F)F)C3)ccc12]

or pharmaceutically acceptable salt or solvate thereof.

In another aspect, provided herein is a method of vector control comprising administering an insecticide to a human; wherein the insecticide is lethal to a vector exposed to the administered insecticide during a bite or blood meal with the human. In some cases, the insecticide is lethal to the vector within 8, 7, 6, 5, 4, 3, 2 or 1 days of exposure. In some cases, the insecticide is an ectoparasiticide. In some cases, the insecticide is an isoxazoline compound. In some cases, the isoxazoline compound has Formula (I)

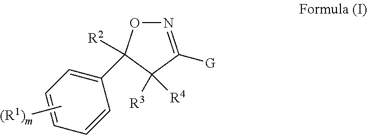

wherein,
each R¹ is independently selected from -D, —OR⁵, —SR⁵, —N(R⁶)(R⁷), —F, —Cl, —Br, —I, —C(O)R⁵, —CO₂R⁵, —CN, —NO₂, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
each R⁵ is independently selected from —H, substituted or unsubstituted C₁-C₆alkyl, substituted or unsubstituted C₃-C₇cycloalkyl, substituted or unsubstituted aryl, and substituted or unsubstituted heteroaryl;
each R⁶ and R⁷ are independently selected from —H, substituted or unsubstituted C₁-C₇alkyl, substituted or unsubstituted C₁-C₇fluoroalkyl, and substituted or unsubstituted C₁-C₇heteroalkyl;
R⁶ and R⁷ can optionally be taken together with the N-atom to which they are attached to form a N-containing heterocycle;
R² is —H, —F, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, substituted or unsubstituted benzyl, or substituted or unsubstituted heteroaryl;
each R³ and R⁴ are independently selected from —H, —F, substituted or unsubstituted C₁-C₇alkyl, substituted or unsubstituted C₁-C₇fluoroalkyl, and substituted or unsubstituted C₁-C₇heteroalkyl; substituted or unsubstituted C₃-C₇cycloalkyl, substituted or unsubstituted C₂-C₆heterocycloalkyl, substituted or unsubstituted aryl, and substituted or unsubstituted heteroaryl;
m is 0, 1, 2, 3, 4, or 5; and
G is substituted or unsubstituted aryl or substituted or unsubstituted heteroaryl.

In some cases, G is

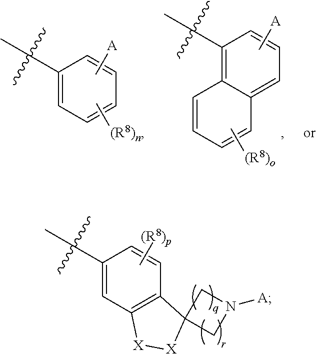

each R⁸ is independently selected from -D, —OR⁵, —SR⁵, —N(R⁶)(R⁷), —F, —Cl, —Br, —I, substituted or unsubstituted C₁-C₇alkyl, substituted or unsubstituted C₂-C₇alkenyl, substituted or unsubstituted C₂-C₇alkynyl, substituted or unsubstituted C₁-C₇fluoroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
two R⁸ groups can optionally be taken together with the adjacent carbon atoms to which they are attached to form aromatic or partially saturated carbocycle or heterocycle;
each X is independently selected from —O—, —S—, —S(═O)—, —S(═O)₂—, —NR⁶—, —C(═O)—, and —(CR⁹R¹⁰)ₛ—, wherein each R⁹ and R¹⁰ are independently selected from —H, D, —F, —OR⁵, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl; s is 1, 2, or 3;
n is 0, 1, 2, 3, or 4;
o is 0, 1, 2, 3, 4, 5, or 6;
p is 0, 1, 2, or 3;
q is 0, 1, or 2;
r is 0, 1, or 2;
A is

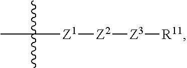

wherein
Z¹, Z², and Z³ are independently absent or selected from —(CR¹²R¹³)ᵤ—, —NR⁶—, —C(═O)—, —S(═O)—, —S(═O)₂—, —C(═O)(CR¹²R¹³)ᵤ—, —(CR¹²R¹³)ᵤC(═O)—, —C(═O)NR⁶—, —NR⁶C(═O)—, —C(═O)O—, —OC(═O)—, —OC(═O)NR⁶—, —NR⁶C(═O)O—, —NR⁶C(═O)NR⁶—, —C(═O)NR⁶(CR¹²R¹³)ᵤ—, —NR⁶C(═O)(CR¹²R¹³)ᵤ—, —(CR¹²R¹³)ᵤC(═O)NR⁶—, and —(CR¹²R¹³)ᵤNR⁶C(═O)—;
each R¹² and R¹³ are independently selected from —H, -D, —F, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl;
u is 1, 2, 3, or 4; and
R¹¹ is substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, or substituted or unsubstituted heteroaryl.

In some cases,

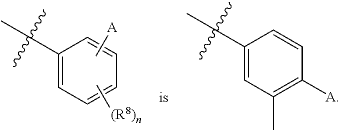

In some cases,

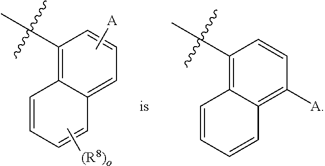

In some cases,

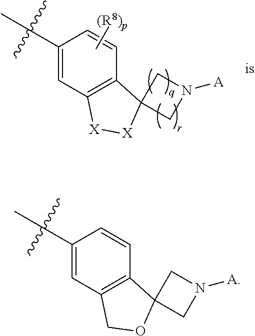

In some cases, A is

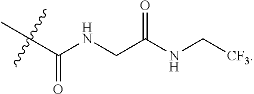 [SMILES: *C(=O)NCC(=O)NCC(F)(F)F]

In some cases, A is

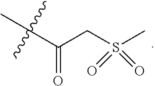 [SMILES: *C(=O)CS(C)(=O)=O]

In some cases, the compound of Formula (I) is fluralaner,

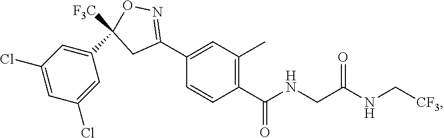 [SMILES: Cc1cc(C2=NO[C@](c3cc(Cl)cc(Cl)c3)(C(F)(F)F)C2)ccc1C(=O)NCC(=O)NCC(F)(F)F]

or pharmaceutically acceptable salt or solvate thereof.

In some cases, the compound of Formula (I) is afoxolaner,

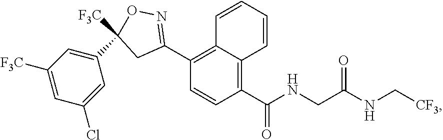 [SMILES: O=C(CNC(=O)c1ccc(C2=NO[C@](c3cc(Cl)cc(C(F)(F)F)c3)(C(F)(F)F)C2)c2ccccc12)NCC(F)(F)F]

or pharmaceutically acceptable salt or solvate thereof.

In some cases, the compound of Formula (I) is (S)-afoxolaner,

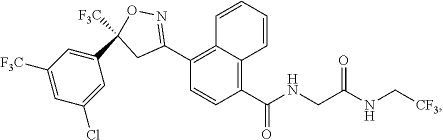 [SMILES: O=C(CNC(=O)c1ccc(C2=NO[C@@](c3cc(Cl)cc(C(F)(F)F)c3)(C(F)(F)F)C2)c2ccccc12)NCC(F)(F)F]

or pharmaceutically acceptable salt or solvate thereof.

In some cases, the compound of Formula (I) is (R)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide,

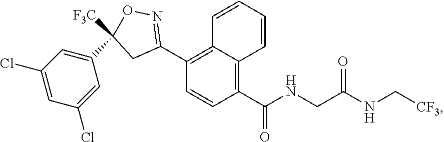 [SMILES: O=C(CNC(=O)c1ccc(C2=NO[C@](c3cc(Cl)cc(Cl)c3)(C(F)(F)F)C2)c2ccccc12)NCC(F)(F)F]

or pharmaceutically acceptable salt or solvate thereof.

In some cases, the compound of Formula (I) is (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide,

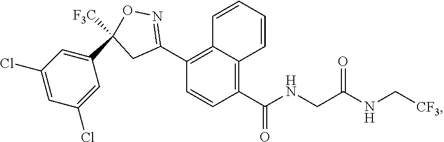 [SMILES: O=C(CNC(=O)c1ccc(C2=NO[C@@](c3cc(Cl)cc(Cl)c3)(C(F)(F)F)C2)c2ccccc12)NCC(F)(F)F]

or pharmaceutically acceptable salt or solvate thereof.

In some cases, the compound of Formula (I) is sarolaner,

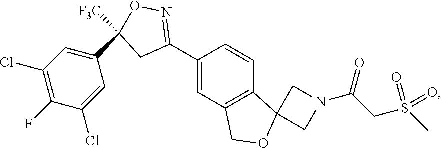 [SMILES: CS(=O)(=O)CC(=O)N1CC2(C1)OCc1cc(C3=NO[C@@](c4cc(Cl)c(F)c(Cl)c4)(C(F)(F)F)C3)ccc12]

or pharmaceutically acceptable salt or solvate thereof.

In some cases, the insecticide comprises fluralaner, afoxolaner, sarolaner, allethrin, resmethrin, phenothrin, etofenprox, permethrin, imidacloprid, fipronil, methoprene, fenoxycarb, pyriproxyfen, lufenuron, diflubenzuron, amitraz, selamectin, nitenpyram, dinotefuran, spinosad, or a pharmaceutically acceptable salt or derivative thereof. In some cases, the insecticide targets the glutamate gated chloride channel. In some cases, the insecticide targets γ-aminobutyric acid (GABA)-gated chloride channel (GABAC1). In some cases, the insecticide targets the γ-aminobutyric acid (GABA)-gated chloride channel in a location distinct from dieldrin. In some cases, the vector has a mutation in the rdl locus conferring resistance to a cyclodiene, lindane, picrotoxinin, other convulsant, or a combination thereof. In some cases, the vector has a mutation in the rdl locus conferring partial resistance to fipronil. In some cases, the cyclodiene is dieldrin. In some cases, the other convulsant comprises BIDN (3,3-bis(trifluoromethyl)bicyclo[2,2,1]heptane-2,2-dicarbonitrile), EBOB (ethynylbicycloorthobenzoate), or a combination thereof.

In some cases, the vector is an insect vector. In some cases, the insect vector is selected from a mosquito, triatomine bug, tsetse fly, and black fly. In some cases, the insect vector is a mosquito of a genus selected from Aedes, Anopheles, Culex, and Phlebotomus. In some cases, the insect vector is a mosquito capable of transmitting a virus selected from a flavivirus, bunyavirus and a togavirus. In some cases, the flavivirus is selected from zika virus, Japanese encephalitis, dengue virus, yellow fever virus, Powassan virus and usutu virus. In some cases, the bunyavirus is selected from Rift Valley fever, Punta Toro virus, La Crosse virus, Maporal virus, Heartland virus, and Severe Fever thrombocytopenia syndrome virus. In some cases, the togavirus is selected from Venezuelan equine encephalitis virus, Eastern equine encephalitis virus, Western equine encephalitis virus, and chikungunya virus. In some cases, the insect vector is the Anopheles mosquito and the Anopheles mosquito is capable of transmitting o′nyong-nyong virus. In some cases, the insect vector is the Anopheles mosquito and the Anopheles mosquito is capable of transmitting a Plasmodium parasite. In some cases, the Plasmodium parasite is selected from P. falciparum, P. malariae, P. ovale, P. vivax and P. knowlesi. In some cases, the Plasmodium parasite causes malaria. In some cases, the insect vector is the Culex mosquito and the Culex mosquito is capable of transmitting a virus selected from Japanese encephalitis virus and West Nile virus. In some cases, the insect vector is the Culex mosquito and the Culex mosquito is capable of transmitting a parasitic nematode. In some cases, the parasitic nematode is Wuchereria bancrofti. In some cases, insect vector is the Phlebotomus sandfly and the Phlebotomus sandfly mosquito is capable of transmitting a Leishmania parasite. In some cases, the insect vector is the Phlebotomus sandfly and the Phlebotomus sandfly is capable of transmitting a virus within the Phlebovirus genus of the Bunyaviridae family. In some cases, the insect vector is the triatomine bug and the triatomine bug is capable of transmitting a Trypanosoma cruzi parasite. In some cases, the insect vector is the tsetse fly and the tsetse fly is capable of transmitting a Trypanosoma brucei parasite. In some cases, the insect vector is the black fly and the black fly is capable of transmitting an Onchocerca volvulus parasite. In some cases, the vector is an ectoparasite. In some cases, the ectoparasite is selected from a tick and a flea. In some cases, the ectoparasite is the tick and the tick is capable of transmitting a virus selected from Crimean-Congo haemorrhagic fever (CCHF) virus and tick-borne encephalitis virus. In some cases, the ectoparasite is the tick and the tick is capable of transmitting a bacterium selected from Borrelia burgdorferi, Borrelia spirochetes, Anaplasma phagocytophilum, Ehrlichia chaffeensis, Ehrlichia muris, Ehrlichia ewingii, Neoehrlichia mikurensis, Rickettsia aeschlimannii, Rickettsia africae, Rickettsia australis, Rickettsia conorii, Rickettsia heilong-jiangensis, Rickettsia helvetica, Rickettsia honei, Rickettsia japonica, Rickettsia massiliae, Rickettsia monacensis, Rickettsia parkeri, Rickettsia raoultii, Rickettsia rickettsii, Rickettsia sibirica, Rickettsia sibiricamongolotimonae, Rickettsia slovaca, and Francisella tularensis. In some cases, the ectoparasite is the flea and the flea is capable of transmitting a bacterium selected from Yersinia pestis, Rickettsia felis, and Rickettsia typhi.

In some cases, the insecticide is administered in an oral dosage form. In some cases, a dose of between 1 mg/kg and 50 mg/kg of the insecticide is administered to the human. In some cases, the insecticide is administered in a single dose, and the single dose is optionally repeated no more than every 3 months. In some cases, the single dose is repeated no more than every 9-12 months. In some cases, the single dose is administered once yearly for a period of 1, 2, 3, 4, 5, 6, 7, 8, 9 or 10 years. In some cases, the single dose is effective in killing the exposed vector at least about 30, 40, 50, 60, 70, 80, 90, 100, 110, 120, 130, 140, 150, 160, 170, 180, 240, or 360 days after administration. In some cases, the insecticide is administered in a single regimen comprising administration of a plurality of doses over a period of 1 week or less. In some cases, the total dose of the insecticide administered in the single regimen is between 1 mg/kg and 50 mg/kg. In some cases, the plurality of doses is 2, 3, 4 or 5 doses. In some cases, the insecticide is administered over a period of 2, 3, 4, 5, 6 or 7 days. In some cases, the single regimen is effective in killing the exposed vector at least about 30, 40, 50, 60, 70, 80, 90, 100, 110, 120, 130, 140, 150, 160, 170, 180, 240, or 360 days after administration.

In some cases, the vector is capable of transmitting a vector-borne disease to the human during a transmission season. In some cases, the insecticide is administered within 30, 20, 10, 5, 4, 3, 2 or 1 day of the beginning of the transmission season. In some cases, the transmission season correlates to a season having collectively an average daily rain fall that is higher than the average daily rain fall for an entire year. In some cases, the transmission season is dependent on rainfall patterns, temperature and/or humidity. In some cases, a calendar year comprises 1 transmission season. In some cases, a calendar year comprises 2 or more transmission seasons. In some cases, the transmission season comprises at least 30, 45, 60, 75, 90, or 120 days. In some cases, the transmission season is less than or equal to about 180, 150, 120, 90, 75 or 60 days. In some cases, the human is administered the insecticide prior to travel or deployment to a region comprising the vector. In some cases, the human resides, works or is deployed within a region comprising the vector. In some cases, there is a risk of the human acquiring a vector-borne disease after the bite or blood meal with the vector. In some cases, the risk is described in the 2016, 2018, or a current edition of CDC Health Information for International Travel book as a high or moderate risk. In some cases, the human is deployed to the region. In some cases, the human is a military member. In some cases, the human is a civilian. In some cases, the human is about 5 years or older in age. In some cases, the human is about 18 years or older in age. In some cases, the human is one of a plurality of individuals in a human population and the plurality of individuals is administered the insecticide within an administration period. In some cases, the plurality of individuals excludes those from the human population having an existing condition, contraindication to the insecticide, pregnant women, children under the age of 5, or a combination thereof. In some cases, the plurality of individuals comprises at least about 50%, 60%, 70%, 80%, 85%, 90% or 95% of the human population. In some cases, the plurality of individuals comprises at least 50% of the human population, and the number of clinically identified disease transmissions between the administration period and about 3 months following the administration period is less than about 50% of the number of clinically identified transmissions for the same 3 month time period in one or more of the previous 10 years. In some cases, the plurality of individuals comprises at least 50% of the human population, and the number of vectors identified between the administration period and about 3 months following the administration period is less than about 50% of the number of vectors identified for the same 3 month time period in one or more of the previous 10 years.

In some cases, the method further comprises administering to the human dihydroartemisinin-piperaquine; artemether and lumefantrine; artesunate and amodiaquine; artesunate and mefloquine; artesunate and sulfadoxine-pyrimethamine; primaquine; quinine and clindamycin; chloroquine; atovoquone/proguanil; or a combination thereof. In some cases, the method further comprises administering to the human ivermectin; albendazole; diethylcarbamazine citrate; ribavirin; pentavalent antimonials; ampthotericin B deoxycholate; paromycin; pentamidine isethionate; miltefosine; azoles medicines; pentamidine; suramin; melarsoprol; elfornithine; nifurtimox; antibiotic; or a combination thereof. In some cases, the antibiotic is doxycycline. In some cases, the human uses a bed-net to avoid the bite or blood meal with the vector. In some cases, the bed-net comprises or is applied with a pyrethroid. In some cases, the method further comprises applying to a region in which the human resides, works or is deployed with an additional insecticide. In some cases, the additional insecticide is a pyrethroid, an organochlorine, an organophosphate, a carbamate, a phenylpyrazole, a pyrrole, a macrocylcic lactone or a meta-diamide.

In another aspect, provided herein is a method of preventing transmission of a disease-causing organism from a vector to a human population, the method comprising administering to each of a plurality of individuals of the population an insecticide in a single dose or single regimen over the course of less than or equal to 7 days; wherein the insecticide is present in one or more of the plurality of individuals at a concentration that is lethal to the vector within 90 days of the vector biting or engaging in a blood meal with the one or more of the plurality of individuals. In some cases, the insecticide is present in the one or more of the plurality of individuals at a concentration that is lethal to the vector within 60 days of the vector biting or engaging in a blood meal with the one or more of the plurality of individuals. In some cases, the insecticide is present in the one or more of the plurality of individuals at a concentration that is lethal to the vector within 30 days of the vector biting or engaging in a blood meal with the one or more of the plurality of individuals. In some cases, the human population resides, works, travels, and/or is deployed within a region comprising the vector. In some cases, at least one of the plurality of individuals is administered the insecticide prior to travel or deployment to the region. In some cases, the plurality of individuals excludes those from the human population having an existing condition that is adverse to the insecticide. In some cases, the plurality of individuals excludes those from the human population that are pregnant. In some cases, the plurality of individuals excludes those from the human population that are nursing. In some cases, the plurality of individuals excludes those from the human population that are children under the age of about 5. In some cases, the plurality of individuals comprises at least about 50%, 60%, 70%, 80%, 85%, 90% or 95% of the human population. In some cases, the plurality of individuals is administered the insecticide within an administration period. In some cases, the administration period is between about 1 day and about 1 month. In some cases, the number of clinically identified disease transmissions within the population that occur between the administration period and about 3 months following the administration period is less than about 50% of the number of clinically identified disease transmissions for the same 3 month time period in one or more of the previous 10 years. In some cases, the number of vectors identified between the administration period and about 3 months following the administration period is less than about 50% of the number of vectors identified for the same 3 month time period in one or more of the previous 10 years.

In some cases, the single dose or single regimen comprises oral administration. In some cases, between 1 mg/kg and 50 mg/kg of the insecticide is administered to the human in the single dose or single regimen. In some cases, the insecticide is administered in the single dose, and the single dose is optionally repeated no more than every 3 months. In some cases, the single dose is repeated no more than every 9-12 months. In some cases, the single dose is administered once yearly for a period of 1, 2, 3, 4, 5, 6, 7, 8, 9 or 10 years. In some cases, the insecticide is administered in the single regimen comprising a plurality of doses. In some cases, the total dose of the insecticide administered in the single regimen is between 1 mg/kg and 50 mg/kg. In some cases, the plurality of doses is 2, 3, 4 or 5 doses. In some cases, the insecticide is administered over a period of 2, 3, 4, 5, 6 or 7 days. In some cases, the vector is capable of transmitting the disease-causing organism to the human population during a transmission season. In some cases, the insecticide is administered within 30, 20, 10, 5, 4, 3, 2 or 1 day of the beginning of the transmission season. In some cases, the transmission season correlates to a season having collectively an average daily rain fall that is higher than the average daily rain fall for an entire year. In some cases, the transmission season is dependent on rainfall patterns, temperature and/or humidity. In some cases, a calendar year comprises 1 transmission season. In some cases, a calendar year comprises 2 or more transmission seasons. In some cases, the transmission season comprises at least 30, 45, 60, 75, 90, or 120 days. In some cases, the transmission season is less than or equal to about 180, 150, 120, 90, 75 or 60 days.

In some cases, the insecticide is an ectoparasiticide. In some cases, the insecticide is an isoxazoline compound. In some cases, the isoxazoline compound has Formula (I)

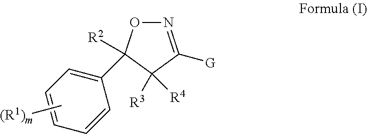

wherein,
each R¹ is independently selected from -D, —OR⁵, —SR⁵, —N(R⁶)(R⁷), —F, —Cl, —Br, —I, —C(O)R⁵, —CO₂R⁵, —CN, —NO₂, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
each R⁵ is independently selected from —H, substituted or unsubstituted C₁-C₆alkyl, substituted or unsubstituted C₃-C₇cycloalkyl, substituted or unsubstituted aryl, and substituted or unsubstituted heteroaryl;
each R⁶ and R⁷ are independently selected from H, substituted or unsubstituted C₁-C₇alkyl, substituted or unsubstituted C₁-C₇fluoroalkyl, and substituted or unsubstituted C₁-C₇heteroalkyl;
R⁶ and R⁷ can optionally be taken together with the N-atom to which they are attached to form a N-containing heterocycle;
R² is —H, —F, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, substituted or unsubstituted benzyl, or substituted or unsubstituted heteroaryl;
each R³ and R⁴ are independently selected from H, —F, substituted or unsubstituted C₁-C₇alkyl, substituted or unsubstituted C₁-C₇fluoroalkyl, and substituted or unsubstituted C₁-C₇heteroalkyl; substituted or unsubstituted C₃-C₇cycloalkyl, substituted or unsubstituted C₂-C₆heterocycloalkyl, substituted or unsubstituted aryl, and substituted or unsubstituted heteroaryl;
m is 0, 1, 2, 3, 4, or 5; and
G is substituted or unsubstituted aryl or substituted or unsubstituted heteroaryl.

In some cases, G is

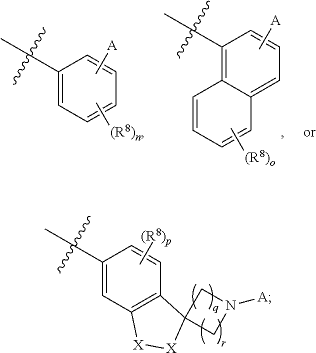

each R⁸ is independently selected from -D, —OR⁵, —SR⁵, —N(R⁶)(R⁷), —F, —Cl, —Br, —I, substituted or unsubstituted C₁-C₇alkyl, substituted or unsubstituted C₂-C₇alkenyl, substituted or unsubstituted C₂-C₇alkynyl, substituted or unsubstituted C₁-C₇fluoroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
two R⁸ groups can optionally be taken together with the adjacent carbon atoms to which they are attached to form aromatic or partially saturated carbocycle or heterocycle;
each X is independently selected from —O—, —S—, —S(═O)—, —S(═O)₂—, —NR⁶—, —C(═O)—, and —(CR⁹R¹⁰)ₛ—, wherein each R⁹ and R¹⁰ are independently selected from —H, -D, —F, —OR⁵, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl; s is 1, 2, or 3;
n is 0, 1, 2, 3, or 4;
o is 0, 1, 2, 3, 4, 5, or 6;
p is 0, 1, 2, or 3;
q is 0, 1, or 2;
r is 0, 1, or 2;

A is

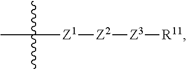

wherein
Z¹, Z², and Z³ are independently absent or selected from —(CR¹²R¹³)ᵤ—, —NR⁶—, —C(═O)—, —S(═O)—, —S(═O)₂—, —C(═O)(CR¹²R¹³)ᵤ—, —(CR¹²R¹³)ᵤC(═O)—, —C(═O)NR⁶—, —NR⁶C(═O)—, —C(═O)O—, —OC(═O)—, —OC(═O)NR⁶—, —NR⁶C(═O)O—, —NR⁶C(═O)NR⁶—, —C(═O)NR⁶(CR¹²R¹³)ᵤ—, —NR⁶C(═O)(CR¹²R¹³)ᵤ—, —(CR¹²R¹³)ᵤC(═O)NR⁶—, and —(CR¹²R¹³)ᵤNR⁶C(═O)—;
each R¹² and R¹³ are independently selected from —H, -D, —F, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl;
u is 1, 2, 3, or 4; and
R¹¹ is substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, or substituted or unsubstituted heteroaryl.

In some cases,

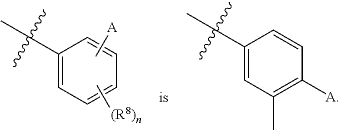

In some cases,

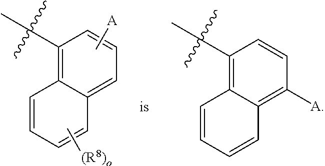

In some cases,

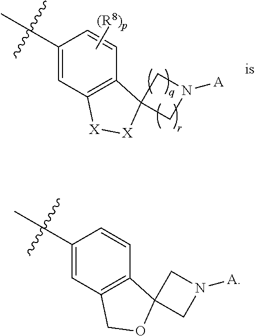

In some cases, A is

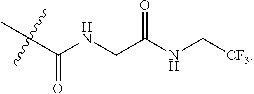 [SMILES: *C(=O)NCC(=O)NCC(F)(F)F]

In some cases, A is

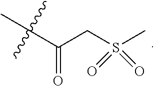 [SMILES: *C(=O)CS(C)(=O)=O]

In some cases, the compound of Formula (I) is fluralaner,

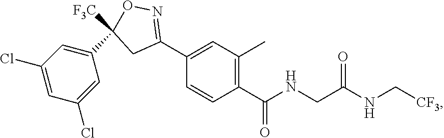 [SMILES: Cc1cc(C2=NO[C@](c3cc(Cl)cc(Cl)c3)(C(F)(F)F)C2)ccc1C(=O)NCC(=O)NCC(F)(F)F]

or pharmaceutically acceptable salt or solvate thereof.

In some cases, the compound of Formula (I) is afoxolaner,

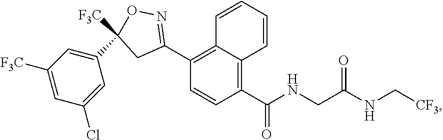 [SMILES: O=C(CNC(=O)c1ccc(C2=NO[C@](c3cc(Cl)cc(C(F)(F)F)c3)(C(F)(F)F)C2)c2ccccc12)NCC(F)(F)F]

or pharmaceutically acceptable salt or solvate thereof.

In some cases, the compound of Formula (I) is (S)-afoxolaner,

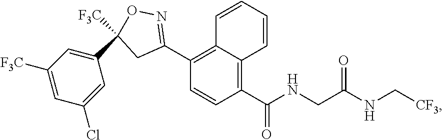 [SMILES: O=C(CNC(=O)c1ccc(C2=NO[C@@](c3cc(Cl)cc(C(F)(F)F)c3)(C(F)(F)F)C2)c2ccccc12)NCC(F)(F)F]

or pharmaceutically acceptable salt or solvate thereof.

In some cases, the compound of Formula (I) is (R)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide,

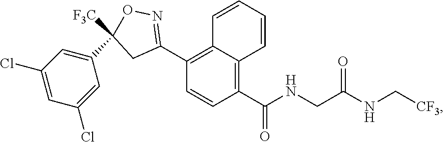 [SMILES: O=C(CNC(=O)c1ccc(C2=NO[C@](c3cc(Cl)cc(Cl)c3)(C(F)(F)F)C2)c2ccccc12)NCC(F)(F)F]

or pharmaceutically acceptable salt or solvate thereof.

In some cases, the compound of Formula (I) is (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide,

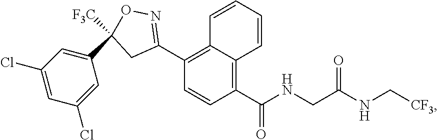 [SMILES: O=C(CNC(=O)c1ccc(C2=NO[C@@](c3cc(Cl)cc(Cl)c3)(C(F)(F)F)C2)c2ccccc12)NCC(F)(F)F]

or pharmaceutically acceptable salt or solvate thereof.

In some cases, the compound of Formula (I) is sarolaner,

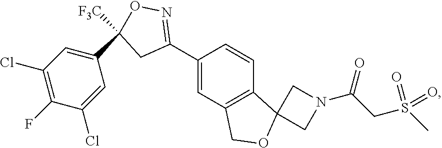 [SMILES: CS(=O)(=O)CC(=O)N1CC2(C1)OCc1cc(C3=NO[C@@](c4cc(Cl)c(F)c(Cl)c4)(C(F)(F)F)C3)ccc12]

or pharmaceutically acceptable salt or solvate thereof.

In some cases, the insecticide comprises fluralaner, afoxolaner, sarolaner, allethrin, resmethrin, phenothrin, etofenprox, permethrin, imidacloprid, fipronil, methoprene, fenoxycarb, pyriproxyfen, lufenuron, diflubenzuron, amitraz, selamectin, nitenpyram, dinotefuran, spinosad, or a pharmaceutically acceptable salt or derivative thereof. In some cases, the insecticide targets the glutamate gated chloride channel. In some cases, the insecticide targets γ-aminobutyric acid (GABA)-gated chloride channel (GABAC1). In some cases, the insecticide targets the γ-aminobutyric acid (GABA)-gated chloride channel in a location distinct from dieldrin. In some cases, the vector has a mutation in the rdl locus conferring resistance to a cyclodiene, lindane, picrotoxinin, other convulsant, or a combination thereof. In some cases, the vector has a mutation in the rdl locus conferring partial resistance to fipronil. In some cases, the cyclodiene is dieldrin. In some cases, the other convulsant comprises BIDN, EBOB, or a combination thereof.

In some cases, the vector is an insect vector. In some cases, the insect vector is selected from a mosquito, triatomine bug, tsetse fly, and black fly. In some cases, the insect vector is a mosquito of a genus selected from Aedes, Anopheles, Culex, and Phlebotomus. In some cases, the insect vector is a mosquito capable of transmitting a virus selected from a flavivirus, bunyavirus and a togavirus. In some cases, the flavivirus is selected from zika virus, Japanese encephalitis, dengue virus, yellow fever virus, Powassan virus and usutu virus. In some cases, the bunyavirus is selected from Rift Valley fever, Punta Toro virus, La Crosse virus, Maporal virus, Heartland virus, and Severe Fever thrombocytopenia syndrome virus. In some cases, the togavirus is selected from Venezuelan equine encephalitis virus, Eastern equine encephalitis virus, Western equine encephalitis virus, and chikungunya virus. In some cases, the insect vector is the Anopheles mosquito and the Anopheles mosquito is capable of transmitting o′nyong-nyong virus. In some cases, the insect vector is the Anopheles mosquito and the Anopheles mosquito is capable of transmitting a Plasmodium parasite. In some cases, the Plasmodium parasite is selected from P. falciparum, P. malariae, P. ovale, P. vivax and P. knowlesi. In some cases, the Plasmodium parasite causes malaria. In some cases, the insect vector is the Culex mosquito and the Culex mosquito is capable of transmitting a virus selected from Japanese encephalitis virus and West Nile virus. In some cases, the insect vector is the Culex mosquito and the Culex mosquito is capable of transmitting a parasitic nematode. In some cases, the parasitic nematode is Wuchereria bancrofti. In some cases, the insect vector is the Phlebotomus sandfly and the Phlebotomus sandfly is capable of transmitting a Leishmania parasite. In some cases, the insect vector is the Phlebotomus sandfly and the Phlebotomus sandfly is capable of transmitting a virus within the Phlebovirus genus of the Bunyaviridae family. In some cases, the insect vector is the triatomine bug and the triatomine bug is capable of transmitting a Trypanosoma cruzi parasite. In some cases, the insect vector is the tsetse fly and the tsetse fly is capable of transmitting a Trypanosoma brucei parasite. In some cases, the insect vector is the black fly and the black fly is capable of transmitting an Onchocerca volvulus parasite. In some cases, the vector is an ectoparasite. In some cases, the ectoparasite is selected from a tick and a flea. In some cases, the ectoparasite is the tick and the tick is capable of transmitting a virus selected from Crimean-Congo haemorrhagic fever (CCHF) virus and tick-borne encephalitis virus. In some cases, the ectoparasite is the tick and the tick is capable of transmitting a bacterium selected from Borrelia burgdorferi, Borrelia spirochetes, Anaplasma phagocytophilum, Ehrlichia chaffeensis, Ehrlichia muris, Ehrlichia ewingii, Neoehrlichia mikurensis, Rickettsia aeschlimannii, Rickettsia africae, Rickettsia australis, Rickettsia conorii, Rickettsia heilong-jiangensis, Rickettsia helvetica, Rickettsia honei, Rickettsia japonica, Rickettsia massiliae, Rickettsia monacensis, Rickettsia parkeri, Rickettsia raoultii, Rickettsia rickettsii, Rickettsia sibirica, Rickettsia sibiricamongolotimonae, Rickettsia slovaca, and Francisella tularensis. In some cases, the ectoparasite is the flea and the flea is capable of transmitting a bacterium selected from Yersinia pestis, Rickettsia felis, and Rickettsia typhi.

In some cases, the method further comprises administering to the plurality of individuals dihydroartemisinin-piperaquine; artemether and lumefantrine; artesunate and amodiaquine; artesunate and mefloquine; artesunate and sulfadoxine-pyrimethamine; primaquine; quinine and clindamycin; chloroquine; atovoquone/proguanil; or a combination thereof. In some cases, the method further comprises administering to the human ivermectin; albendazole; diethylcarbamazine citrate; ribavirin; pentavalent antimonials; ampthotericin B deoxycholate; paromycin; pentamidine isethionate; miltefosine; azoles medicines; pentamidine; suramin; melarsoprol; elfornithine; nifurtimox; antibiotic; or a combination thereof. In some cases, the antibiotic comprises doxycycline. In some cases, one or more members of the human population uses a bed-net to avoid the bite or blood meal with the vector. In some cases, the bed-net comprises or is applied with a pyrethroid.

### BRIEF DESCRIPTION OF THE DRAWINGS

The foregoing summary, as well as the following detailed description of the disclosure, will be better understood when read in conjunction with the appended figures. It should be understood, however, that the disclosure is not limited to the precise examples shown. It is emphasized that, according to common practice, the various features of the drawings are not to-scale. On the contrary, the dimensions of the various features are arbitrarily expanded or reduced for clarity. Included in the drawings are the following figures.

FIG. 1 shows a structural model (left panel) and a cartoon model (right panel) of isoxazoline, ivermectin and fipronil binding to a GABA receptor.

FIG. 2A shows killing of Anopheles stephensi mosquitos fed with various concentrations of fluralaner in a membrane feeding assay.

FIG. 2B shows killing of Anopheles stephensi mosquitos fed with various concentrations of afoxolaner in a membrane feeding assay.

FIG. 2C shows killing of Anopheles stephensi mosquitos fed with various concentrations of fluralaner in another membrane feeding assay.

FIG. 2D shows killing of Anopheles stephensi mosquitos fed with various concentrations of afoxolaner in another membrane feeding assay.

FIG. 2E shows killing of Anopheles stephensi mosquitos fed with various concentrations of (S)- and (R)-enantiomers of afoxolaner.

FIG. 2F shows killing of Anopheles stephensi mosquitos fed with various concentrations of (S)- and (R)-enantiomers of 4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide.

FIG. 3A shows a plot of percentage survival of Kisumu and Tiassale strains of Anopheles gambiae after feeding with various concentrations of dieldrin.

FIG. 3B shows a plot of Kisumu and Tiassale strain survival after feeding with 10 μM dieldrin.

FIG. 4A shows a plot of percentage survival of Kisumu and Tiassale strains of Anopheles gambiae after feeding with various concentrations of fluralaner.

FIG. 4B shows a plot of percentage survival of Kisumu and Tiassale strains of Anopheles gambiae after feeding with various concentrations of afoxolaner.

FIG. 5A shows a plot of percentage survival of New Orleans and Cayman strains of Aedes aegypti after feeding with various concentrations of fluralaner.

FIG. 5B shows a plot of percentage survival of New Orleans and Cayman strains of Aedes aegypti after feeding with various concentrations of afoxolaner.

FIG. 6A shows long intrinsic pharmacokinetic properties of fluralaner in Beagle dogs at 54 mg/kg p.o. QD. This graph is adapted from data in Walther et al., Parasites & Vectors 8:508.

FIG. 6B shows long intrinsic pharmacokinetic properties of afoxolaner in Beagle dogs at 4 mg/kg p.o. QD. This graph is adapted from data in Letendre et al., Veterinary Parasitology, 201, 190-197.

FIG. 7 shows a pharmacokinetic profile of fluralaner and afoxolaner modeled by extrapolating pharmacokinetic curves from published studies in Beagle dogs to the killing concentrations determined by the membrane feeding studies described in Example 2. Data for fluralaner in Beagle dogs was obtained from Walther et al., Parasites & Vectors 8:508. Data from afoxolaner in Beagle dogs was obtained from Letendre et al., Veterinary Parasitology, 201, 190-197.

FIG. 8A shows a modeled impact of a mass drug administration with isoxazoline versus no intervention on prevalence of malaria as identified by microscopy.

FIG. 8B shows a modeled impact of a mass drug administration with isoxazoline versus no intervention on clinical incidence of malaria.

FIG. 9A shows a modeled impact of a mass drug administration with isoxazoline and dihydroartemisinin-piperaquine (DHA-P) versus no intervention on prevalence of malaria as identified by microscopy.

FIG. 9B shows a modeled impact of a mass drug administration with isoxazoline and DHA-P versus no intervention on clinical incidence of malaria.

FIG. 10 shows a modeled impact of mass drug administration of an isoxazoline drug. Reduction in infection incidence (both symptomatic (clinical) and asymptomatic infections) in Zika (panel A) and clinical incidence and cumulative clinical incidence in malaria (panel B) after two years of fluralaner/afoxolaner mass drug administration (MDA) during the transmission season (indicated shaded areas) where either 30% or 80% of the population over the age of 5 received the drug each year. The model assumes a mosquitocidal drug dose resulting in blood levels >IC99 for 90 days.

FIG. 11 shows predicted impact of mass drug administration of an isoxazoline drug on malaria incidence in Africa. The figure shows cumulative reduction in incidence during 2 years of fluralaner/afoxolaner mass drug administration covering 80% of the population over the age of 5 and dosed once per year optimally timed in relation to the start of the transmission season. The model integrates available data on regional disease prevalence and seasonality profile as illustrated in extended FIG. 10.

FIG. 12A shows a plot of percentage survival (mortality) of L. longipalpis after feeding with various concentrations of fluralaner.

FIG. 12B shows a plot of percentage survival (mortality) of L. longipalpis after feeding with various concentrations of afoxolaner.

FIG. 12C shows a plot of percentage survival (mortality) of P. argentipes after feeding with various concentrations of fluralaner.

FIG. 12D shows a plot of percentage survival (mortality) of P. argentipes after feeding with various concentrations of afoxolaner.

FIG. 13A shows a plot of viable Brugia pahangi 24 hours after feeding with fluralaner, afoxolaner, or control compound (moxidectin and ivermectin).

FIG. 13B shows a plot of viable Brugia pahangi 48 hours after feeding with fluralaner, afoxolaner, or control compound (moxidectin and ivermectin).

FIG. 13C shows a plot of viable Brugia pahangi 72 hours after feeding with fluralaner, afoxolaner, or control compound (moxidectin and ivermectin).

### DETAILED DESCRIPTION OF THE INVENTION

In one aspect of the disclosure, provided are methods of vector control involving administration to a human an insecticide that is lethal to a vector after exposure to blood from the human. Exemplary insecticides are of the isoxazoline class and include those which are marketed for administration to animals for control of ectoparasites such as ticks and fleas because they are selectively toxic to insects over mammals. Isoxazoline insecticides are antagonists of the γ-aminobutyric acid (GABA) receptor of the chloride channel at a distinct binding site from conventional insecticides such as avermectins and fipronil. Non-limiting examples of isoxazoline insecticides useful for the methods described herein include fluralaner, afoxolaner, sarolaner, and derivatives thereof.

Methods of vector control include mass drug administration (MDA) of an insecticide to members of a population at risk of spreading vector-borne diseases. The insecticide may be formulated for long-acting use and thus can be administered in a single dose or regimen that is effective for periods of weeks, months or longer. For example, for vectors prevalent during a particular season of the year, members of an at-risk population are administered the insecticide in a single dose or regimen prior to or at the beginning of the season. After partaking in a blood meal from those receiving the insecticide, the vectors are killed and thus cannot spread the disease causing organisms to other humans or hosts. Such methods may be useful for a variety of populations at risk of vector-borne diseases such as malaria, Zika, West-Nile fever, dengue fever and yellow fever.

Before the present methods and compositions are described, it is to be understood that this disclosure is not limited to a particular method or composition described, as such may vary. It is also to be understood that the terminology used herein is for the purpose of describing particular embodiments only, and is not intended to be limiting, since the scope of the present disclosure will be limited only by the appended claims. Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a bacterium” includes a plurality of such bacteria and reference to “the compound” includes reference to one or more compounds and derivatives or analogs thereof known to those skilled in the art, and so forth. As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### Insecticide Compounds

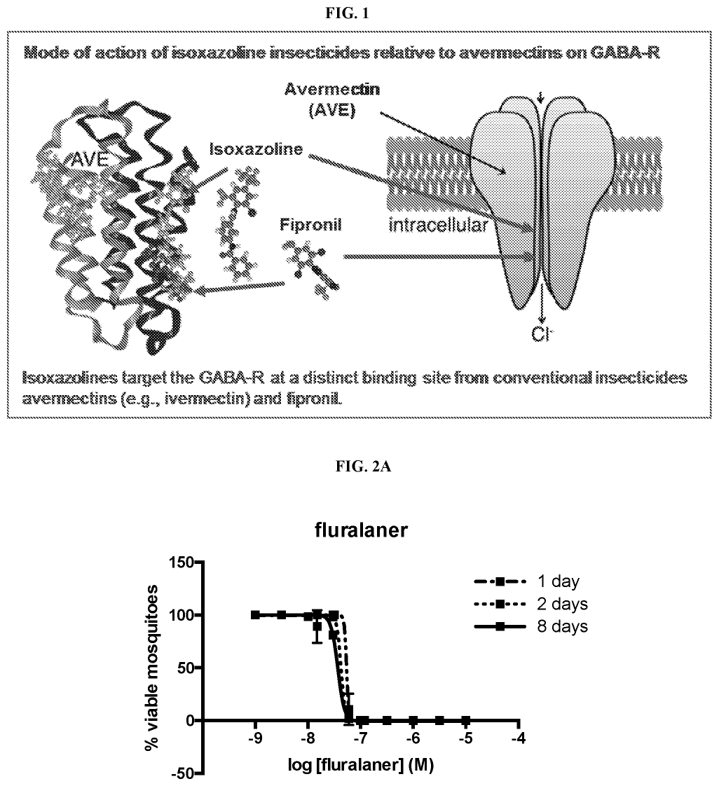

Provided herein are insecticide compounds for human administration. Such insecticide compounds may be present in the human at concentrations lethal to a vector that bites or engages in a blood meal with the human after the administration. In some cases, the insecticide functions by targeting GABA-R in the vector. FIG. 1 shows differential targeting of insecticide compounds to GABA-R. The insecticide may be of the isoxazoline class, which may target a different location of GABA-R than avermectins and/or fipronil. The insecticide may be derived from an isoxazoline compound. Such derivatives include those which are amenable for formulation into a composition for administration to a human.

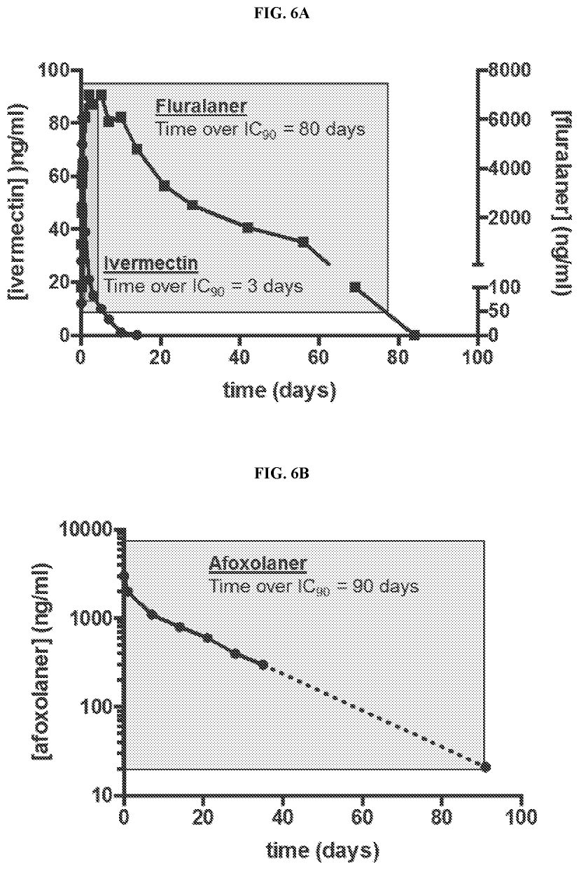

An insecticide compound may have long intrinsic pharmacokinetic properties. As non-limiting examples, the isoxazoline compounds fluralaner and afoxolaner have been shown to have a long half-life in Beagle dogs as evidenced by published pharmacokinetic (PK) data at doses used for tick/flea control (fluralaner: 54 mg/kg by mouth daily, afoxolaner: 4 mg/kg by mouth daily). At these doses, the isoxazoline compound levels are above the mosquitocidal IC₉₀ for 80 days or more as illustrated in FIG. 6A and FIG. 6B (adapted from Walther et al., Parasites & Vectors 8:508; Letendre et al., Veterinary Parasitology, 201, 190-197).

An exemplary insecticide is an isoxazoline compound having Formula (I)

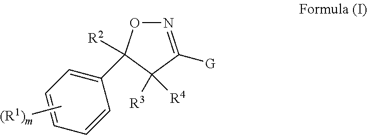

wherein,
each R¹ is independently selected from -D, —OR⁵, —SR⁵, —N(R⁶)(R⁷), —F, —Cl, —Br, —I, —C(O)R⁵, —CO₂R⁵, —CN, —NO₂, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
   each R⁵ is independently selected from H, substituted or unsubstituted C₁-C₆alkyl, substituted or unsubstituted C₃-C₇cycloalkyl, substituted or unsubstituted aryl, and substituted or unsubstituted heteroaryl;
   each R⁶ and R⁷ are independently selected from H, substituted or unsubstituted C₁-C₇alkyl, substituted or unsubstituted C₁-C₇fluoroalkyl, and substituted or unsubstituted C₁-C₇heteroalkyl;
   R⁶ and R⁷ can optionally be taken together with the N-atom to which they are attached to form a N-containing heterocycle;
R² is —H, —F, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, substituted or unsubstituted benzyl, or substituted or unsubstituted heteroaryl;
each R³ and R⁴ are independently selected from —H, —F, substituted or unsubstituted C₁-C₇alkyl, substituted or unsubstituted C₁-C₇fluoroalkyl, and substituted or unsubstituted C₁-C₇heteroalkyl; substituted or unsubstituted C₃-C₇cycloalkyl, substituted or unsubstituted C₂-C₆heterocycloalkyl, substituted or unsubstituted aryl, and substituted or unsubstituted heteroaryl;
m is 0, 1, 2, 3, 4, or 5; and
G is substituted or unsubstituted aryl or substituted or unsubstituted heteroaryl.

In some embodiments, G is

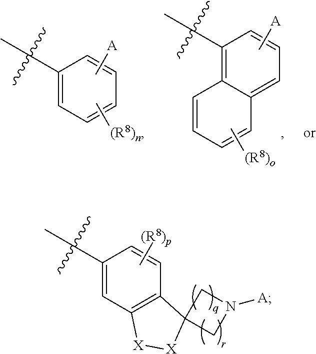

each R⁸ is independently selected from -D, —N(R⁶)(R⁷), —F, —Cl, —Br, —I, substituted or unsubstituted C₁-C₇alkyl, substituted or unsubstituted C₂-C₇alkenyl, substituted or unsubstituted C₂-C₇alkynyl, substituted or unsubstituted C₁-C₇fluoroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
two R⁸ groups can optionally be taken together with the adjacent carbon atoms to which they are attached to form aromatic or partially saturated carbocycle or heterocycle;
each X is independently selected from —O—, —S—, —S(═O)—, —S(═O)₂—, —NR⁶—, —C(═O)—, and —(CR⁹R¹⁰)ₛ—, wherein each R⁹ and R¹⁰ are independently selected from —H, -D, —F, —OR⁵, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl; s is 1, 2, or 3;
n is 0, 1, 2, 3, or 4;
o is 0, 1, 2, 3, 4, 5, or 6;
p is 0, 1, 2, or 3;
q is 0, 1, or 2;
r is 0, 1, or 2;
A is

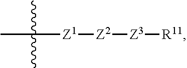

wherein
Z¹, Z², and Z³ are independently absent or selected from —(CR¹²R¹³)ᵤ—, —NR⁶—, —C(═O)—, —S(═O)—, —S(═O)₂—, —C(═O)(CR¹²R¹³)ᵤ—, —(CR¹²R¹³)ᵤC(═O)—, —C(═O)NR⁶—, —NR⁶C(═O)—, —C(═O)O—, —OC(═O)—, —OC(═O)NR⁶—, —NR⁶C(═O)O—, —NR⁶C(═O)NR⁶—, —C(═O)NR⁶(CR¹²R¹³)ᵤ—, —NR⁶C(═O)(CR¹²R¹³)ᵤ—, —(CR¹²R¹³)ᵤC(═O)NR⁶—, and —(CR¹²R¹³)ᵤNR⁶C(═O)—;
   each R¹² and R¹³ are independently selected from —H, -D, —F, —OR⁵, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl;
   u is 1, 2, 3, or 4; and
R¹¹ is substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, or substituted or unsubstituted heteroaryl.

In some embodiments,

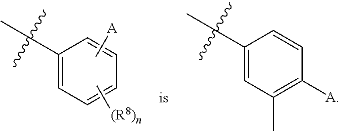

In some embodiments,

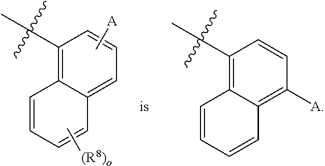

In some embodiments,

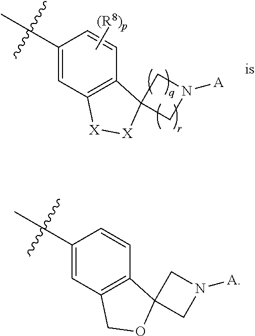

In some embodiments, A is

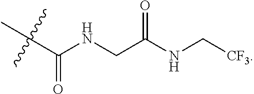 [SMILES: *C(=O)NCC(=O)NCC(F)(F)F]

In some embodiments, A is

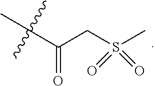 [SMILES: *C(=O)CS(C)(=O)=O]

In some embodiments, the compound of Formula (I) is fluralaner,

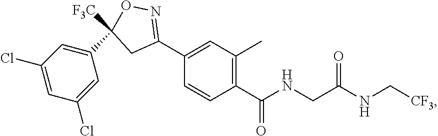 [SMILES: Cc1cc(C2=NO[C@](c3cc(Cl)cc(Cl)c3)(C(F)(F)F)C2)ccc1C(=O)NCC(=O)NCC(F)(F)F]

or pharmaceutically acceptable salt or solvate thereof.

In some cases, the compound of Formula (I) is (S)-fluralaner,

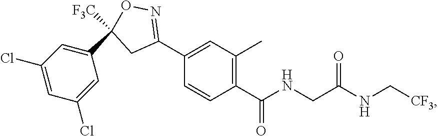 [SMILES: Cc1cc(C2=NO[C@@](c3cc(Cl)cc(Cl)c3)(C(F)(F)F)C2)ccc1C(=O)NCC(=O)NCC(F)(F)F]

or pharmaceutically acceptable salt or solvate thereof.

In some embodiments, the compound of Formula (I) is afoxolaner,

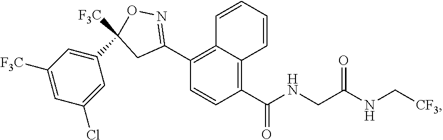 [SMILES: O=C(CNC(=O)c1ccc(C2=NO[C@](c3cc(Cl)cc(C(F)(F)F)c3)(C(F)(F)F)C2)c2ccccc12)NCC(F)(F)F]

or pharmaceutically acceptable salt or solvate thereof.

In some cases, the compound of Formula (I) is (S)-afoxolaner,

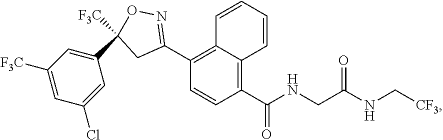 [SMILES: O=C(CNC(=O)c1ccc(C2=NO[C@@](c3cc(Cl)cc(C(F)(F)F)c3)(C(F)(F)F)C2)c2ccccc12)NCC(F)(F)F]

or pharmaceutically acceptable salt or solvate thereof.

In some cases, the compound of Formula (I) is (R)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide,

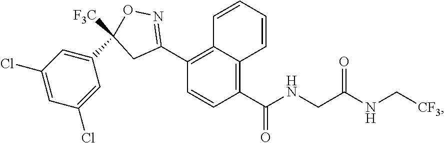 [SMILES: O=C(CNC(=O)c1ccc(C2=NO[C@](c3cc(Cl)cc(Cl)c3)(C(F)(F)F)C2)c2ccccc12)NCC(F)(F)F]

or pharmaceutically acceptable salt or solvate thereof.

In some cases, the compound of Formula (I) is (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide,

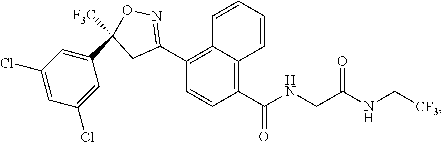 [SMILES: O=C(CNC(=O)c1ccc(C2=NO[C@@](c3cc(Cl)cc(Cl)c3)(C(F)(F)F)C2)c2ccccc12)NCC(F)(F)F]

or pharmaceutically acceptable salt or solvate thereof.

In some embodiments, the compound of Formula (I) is sarolaner,

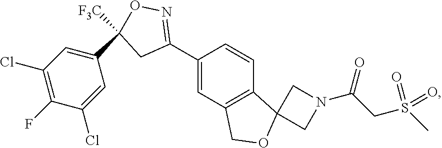 [SMILES: CS(=O)(=O)CC(=O)N1CC2(C1)OCc1cc(C3=NO[C@@](c4cc(Cl)c(F)c(Cl)c4)(C(F)(F)F)C3)ccc12]

or pharmaceutically acceptable salt or solvate thereof.

In some cases, the compound of Formula (I) is (S)-sarolaner,

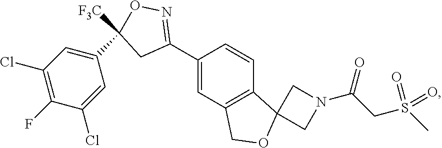 [SMILES: CS(=O)(=O)CC(=O)N1CC2(C1)OCc1cc(C3=NO[C@](c4cc(Cl)c(F)c(Cl)c4)(C(F)(F)F)C3)ccc12]

or pharmaceutically acceptable salt or solvate thereof.

The insecticide compounds disclosed herein, including the compounds of Formula (I), may be prepared by methods known in the field of synthetic chemistry. A compound of Formula (I), or a pharmaceutically acceptable salt, solvate or prodrug thereof, may be formulated with a pharmaceutically acceptable excipient in a pharmaceutical composition. The pharmaceutical composition may be used in a mass drug administration program for vector control. In some cases, the pharmaceutical composition is combined with another insecticide specific for the vector and/or an additional active agent that targets an infectious organism transferred by the vector. In some cases, the pharmaceutical composition further includes the insecticide and/or additional active agent.

In the preceding description of insecticide compounds suitable for use in the methods described herein, definitions of referred-to standard chemistry terms may be found in reference works (if not otherwise defined herein), including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the ordinary skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients.

### Vectors, Organisms, and Infectious Diseases

Insecticide compounds provided herein may be effective in killing a vector exposed to the insecticide during a blood meal with a human treated with the insecticide. Death may occur within 1, 2, 3, 4, 5, 6 or 7 days after feeding on a treated human. Vectors include any organism that carries and transmits an infectious pathogen between subjects. Vectors include arthropods, such as mosquitos, fleas, ticks, lice and mites. Vectors also include the triatomine bug, tsetse fly and black fly. While specific embodiments herein describe insecticide compounds that are useful for killing a vector that transmits a disease, other non-disease transmitting organisms may also be killed by the insecticide. In some such cases, the vector is of the family Cimidicae, e.g., a bed bug.

In some cases, the vector is a mosquito of a genus selected from Aedes, Anopheles, Culex, and Phlebotomus sandflies. In some cases, the vector is the Aedes mosquito and the Aedes mosquito is capable of transmitting a virus selected from a flavivirus, bunyavirus, and a togavirus. In some cases, the vector is capable of transmitting a flavivirus. Non-limiting examples of flaviviruses include zika virus, Japanese encephalitis, dengue virus, yellow fever virus, Powassan virus and usutu virus. In some cases, the vector is capable of transmitting a virus of the bunyaviridae family, such as Rift Valley fever, Punta Toro virus, La Crosse virus, Maporal virus, Heartland virus, Severe Fever thrombocytopenia syndrome virus, or a combination thereof. In some cases, the vector is capable of transmitting a virus of the togaviridae family, such as Venezuelan equine encephalitis virus, Eastern equine encephalitis virus, Western equine encephalitis virus, chikungunya virus, or a combination thereof.

In some cases, the vector is the Aedes mosquito and the Aedes mosquito is capable of transmitting a parasitic nematode. In some cases, the parasitic nematode is Brugia malayi, Brugia pahangi, and Brugia timori. In some cases, the vector is the Anopheles mosquito and the Anopheles mosquito is capable of transmitting a Plasmodium parasite. In some cases, the Plasmodium parasite is selected from P. falciparum, P. malariae, P. ovale, P. vivax and P. knowlesi. In some cases, the Plasmodium parasite causes malaria. In some cases, the vector is the Anopheles mosquito and the Anopheles mosquito is capable of transmitting o′nyong-nyong virus. In some cases, the vector is the Culex mosquito and the Culex mosquito is capable of transmitting a virus selected from Japanese encephalitis virus and West Nile virus. In some cases, the vector is the Culex mosquito and the Culex mosquito is capable of transmitting a parasitic nematode. In some cases, the parasitic nematode is Wuchereria bancrofti. In some cases, the vector is the Phlebotomus sandfly mosquito and the Phlebotomus sandfly mosquito is capable of transmitting a Leishmania parasite. In some cases, the vector is the Phlebotomus sandfly mosquito and the Phlebotomus sandfly mosquito is capable of transmitting a virus within the Phlebovirus genus of the Bunyaviridae family.

In some cases, the vector is the triatomine bug and the triatomine bug is capable of transmitting a Trypanosoma cruzi parasite.

In some cases, the vector is the tsetse fly and the tsetse fly is capable of transmitting a Trypanosoma brucei parasite.

In some cases, the vector is the black fly and the black fly is capable of transmitting an Onchocerca volvulus parasite.

In some cases, the vector is an ectoparasite. In some cases, the ectoparasite is selected from a tick and a flea. In some cases, the ectoparasite is the tick and the tick is capable of transmitting a virus selected from Crimean-Congo haemorrhagic fever (CCHF) virus and tick-borne encephalitis virus. In some cases, the ectoparasite is the tick and the tick is capable of transmitting a bacterium selected from Borrelia burgdorferi, Borrelia spirochetes, Anaplasma phagocytophilum, Ehrlichia chaffeensis, Ehrlichia muris, Ehrlichia ewingii, Neoehrlichia mikurensis, Rickettsia aeschlimannii, Rickettsia africae, Rickettsia australis, Rickettsia conorii, Rickettsia heilong-jiangensis, Rickettsia helvetica, Rickettsia honei, Rickettsia japonica, Rickettsia massiliae, Rickettsia monacensis, Rickettsia parkeri, Rickettsia raoultii, Rickettsia rickettsii, Rickettsia sibirica, Rickettsia sibiricamongolotimonae, Rickettsia slovaca, and Francisella tularensis. In some cases, the ectoparasite is the flea and the flea is capable of transmitting a bacterium selected from Yersinia pestis, Rickettsia felis, and Rickettsia typhi.

### Methods of Use

In one aspect of the disclosure, provided are insecticide compounds as drugs for vector transmission control. The vectors may be killed after exposure to an insecticide compound administered to a human subject. Although the subject may become infected with a disease causing organism from the vector, the resulting killing of the vector will avoid further dissemination of the organism in the region. If the subject is not capable of taking the insecticide, for example, the subject is a small child or is susceptible to an adverse reaction, those living in the vicinity of the subject may be administered the insecticide. For instance, some methods provided herein involve mass drug administration (MDA), where a threshold number of a human population are administered an insecticide compound. In non-limiting examples, a plurality of individuals making up a subset of the human population are administered the insecticide. In some cases, the plurality of individuals make up at least about 50%, 60%, 70%, 80%, 85%, 90% or 95% of the human population. The plurality of individuals may include those that are about 5, 6, 7, 8, 9 or 10 years of age or older. The plurality of individuals may exclude those having an existing condition, contraindication to the insecticide, pregnant women, nursing women, children under the age of 5, or any combination thereof.

The insecticide compound may be administered to individuals regardless of symptoms or disease, eliminating the need for diagnosis. In many cases, the insecticide is orally administered. Accordingly, the MDA may be performed in rural areas without health clinics.

The insecticide compound may be singly administered or administered over a span one or more days. For example, the insecticide compound is administered in a single-dose or in one or more doses over the course of 1, 2, 3, 4, 5, 6, or 7 days. The one or more doses may make up a single regimen.

The insecticide compound may be administered at the beginning of the transmission season. This includes the beginning of the rainy season, which includes 8, 7, 6, 5, 4, 3, 2, or 1 weeks before, or 7, 6, 5, 4, 3, 2, or 1 day before the rainy season. The timing of administration may be selected depending on the elimination kinetics of the insecticide compound and/or the duration of the transmission season. A transmission season may depend on the weather, for instance, prevalence of rain. In some embodiments, for a transmission season which last less than about 120, 110, 100, 90, 80, 70, 60, 50, 40 or 30 days, the insecticide compound is administered once during or before the transmission season, in a single-dose or in or more doses over the course of less than or equal to about 7 days. The MDA may span one or more transmission seasons, optionally with modifications to the regimen, e.g., dosage and/or population, between seasons. One or more transmission seasons may include 2, 3, 4, 5, 6, 7, 8, 9, 10 or more transmission seasons.

An insecticide compound described herein may have a half-life of at least about 10, 20, 30, 40, 50, 60, 70, 80, 90, 100, 110, 120 or 150 days. In some cases, the insecticidal concentration of active agent in human is present at least about 10, 20, 30, 40, 50, 60, 70, 80, 90, 100, 110, 120 or 150 days after insecticide compound administration. Such administration includes a single-dose and multiple-dose administered over a period of time of less than about a week.

The methods described herein are exemplified for human use, however non-human subjects may be administered an insecticide compound herein. As used herein, a “subject” means an animal, such as a mammal, including humans, other higher primates, lower primates, and animals of veterinary importance, such as dogs, cats, horses, sheep, goats, cattle and the like.

Administration frequencies for a pharmaceutical composition of an insecticide compound having Formula (I) and/or one or more additional active agents may vary based on the method being practiced, the physical characteristics of the subject, the identity of vector targeted, and the formulation and the means used to administer the compound. Administration frequencies may include 6, 5, 4, 3, 2 or once daily, every other day, every third day, every fourth day, every fifth day, every sixth day, once weekly, every eighth day, every ninth day, every tenth day, bi-weekly, monthly, every 3 months, every 6 months, every 12 months, or every transmission season. In certain aspects, the pharmaceutical composition is administered once per transmission season in a single dose or single regimen. The duration and/or dosage of administration may be based on the vector to be killed. Under some conditions, administration is continued for a number of transmission seasons and/or years. Under some conditions, a pharmaceutical composition is administered in one, two or three doses over an administration period. In certain aspects, complete administration can be achieved using a single dose of the pharmaceutical composition.

Each of the methods may also be practiced by administering an additional active agent to the subject. Such additional active agents may be included in a pharmaceutical formulation comprising a compound of Formula (I) and/or an insecticide compound, or the additional active agent may be administered separately, whether concurrently or sequentially, in either order. A wide range of additional active agents can be used in combination with the compounds, compositions and methods described herein. An additional active agent may act by preventing the survival, the reproduction or the development of the pathogen in the human host. A non-limiting list of additional active agents includes: artemisinin-based combination therapies such as dihydroartemisinin-piperaquine (DHA-P), artemether and lumefantrine, artesunate and amodiaquine, artesunate and mefloquine, and artesunate and sulfadoxine-pyrimethamine; primaquine; quinine and clindanycin; chloroquine; atovoquone/proguanil; ivermectin; albendazole; diethylcarbamazine citrate; ribavirin; pentavalent antimonials; ampthotericin B deoxycholate; paromycin; pentamidine isethionate; miltefosine; azoles medicines; pentamidine; suramin; melarsoprol; elfornithine; nifurtimox; and antibiotics such as doxycycline.

In some cases, an insecticide compound may be administered in combination with one or more additional means of disease prevention. In some cases, this combination is part of the MDA. Such methods may include administration of an additional active agent, as well as use of environmental control activities such as bed nets and/or spraying of an additional active agent. In some instances, the bed nets are treated with an additional active agent. In some cases, an additional active agent is a prophylaxis and/or therapeutic agent for a disease transmitted by the vector targeted by the insecticide compound.

Mass drug administration methods described herein involve administration of a pharmaceutical composition that includes at least one compound of Formula (I) or a pharmaceutically acceptable salt, pharmaceutically acceptable prodrug, or pharmaceutically acceptable solvate thereof in therapeutically effective amounts, and/or a therapeutically effective amount of an additional active agent to a plurality of individuals of a population. A therapeutically effective amount may be an amount of an insecticide compound that when administered to an individual remains at concentrations in the individual sufficient to be lethal to a vector after the vector ingests or is otherwise exposed to the insecticide through biting. The insecticide may be lethal to a vector within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 140, 150, 160, 180, 200, 220, 240, 260, 280, 300, 320, 340 or 360 days after insecticide compound administration to the individual. Amounts of insecticide compound effective for this use depend on the vector, the individual's health status, weight, and response to the compound, and/or the judgment of the treating physician. Therapeutically effective amounts may be optionally determined by methods including, but not limited to, a dose escalation clinical trial.

The amount of a given agent (e.g., insecticide compound, additional active agent) administered varies depending upon factors such as the particular compound, identity of vector targeted, the identity (e.g., weight, sex) of the individual human or other subject, but can nevertheless be determined according to the particular circumstances surrounding the case, including, e.g., the specific agent being administered, the route of administration, the vector being targeted, and the subject being treated. In general, however, doses employed for adult human treatment are typically in the range of 0.01 mg-5000 mg per single dose or total dose in a single regimen. In one aspect, doses employed for adult human treatment are from about 1 mg to about 1000 mg per single dose or total dose in a single regimen. In one embodiment, the single regimen is presented in divided doses administered simultaneously (or over a short period of time) or at appropriate intervals, for example as two, three, four or more sub-doses per day.

In one embodiment, dosages appropriate for a compound of Formula (I) described herein are from about 0.01 to about 10 mg/kg per body weight. In specific embodiments, an indicated dosage in a large mammal, including, but not limited to, humans, is in the range from about 0.5 mg to about 1000 mg, conveniently administered in divided doses, including, but not limited to, up to four times a day over a single regimen. In one embodiment, the dosage is administered in extended release form. In certain embodiments, suitable unit dosage forms for oral administration comprise from about 1 to 1000 mg active ingredient. In other embodiments, the dosage or the amount of active in the dosage form are lower or higher than the ranges indicated herein, based on a number of variables in regard to an individual treatment regimen. In various embodiments, the daily and unit dosages are altered depending on a number of variables including, but not limited to, the activity of the compound used, the vector to be targeted, the mode of administration, the requirements of the individual subject, and the judgment of the practitioner.

Toxicity and therapeutic efficacy of such regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

### Pharmaceutical Compositions and Formulations

Disclosed herein are insecticide compounds having Formula (I), formulated into pharmaceutical compositions. Also disclosed herein are additional active agents, such as DHA-P, formulated into pharmaceutical compositions. Further disclosed herein is an insecticide compound having Formula (I) and an additional active agent, formulated into a pharmaceutical composition. The pharmaceutical composition may comprise fluralaner, afoxolaner, sarolaner or a combination thereof. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure.

Provided herein are pharmaceutical compositions that include a compound of Formula (I), and/or an additional active agent, and at least one pharmaceutically acceptable inactive ingredient. In some embodiments, the compounds described herein are administered as pharmaceutical compositions in which compounds of Formula (I), and/or additional active agent, are mixed with other active ingredients, as in combination therapy. In other embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In yet other embodiments, the pharmaceutical compositions include other therapeutically valuable substances.

A pharmaceutical composition, as used herein, refers to a mixture of a compound of Formula (I), and/or additional active agent, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to a subject. In practicing the methods of treatment or use provided herein, therapeutically effective amounts of compounds described herein are administered in a pharmaceutical composition to a subject in a population exposed to a vector harboring a disease-causing organism. In some embodiments, the subject is a human. A therapeutically effective amount can vary widely depending on the severity of the vector, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound of Formula (I), and/or additional active agent, are manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound of Formula (I), and/or additional active agent, as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, compounds of Formula (I) and/or additional active agent, exist in unsolvated form or in solvated forms with pharmaceutically acceptable solvents such as water, ethanol, and the like. The solvated forms of the compounds of Formula (I) and/or additional active agent, are also considered to be disclosed herein.

In some embodiments, the compounds of Formula (I), and/or additional active agent, exist as tautomers. All tautomers are included within the scope of the compounds presented herein. As such, it is to be understood that a compounds of the Formula (I), and/or additional active agent, or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the present disclosure encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein.

In some embodiments, compounds of Formula (I), and/or additional active agent, exist as enantiomers, diastereomers, or other steroisomeric forms. The compounds disclosed herein include all enantiomeric, diastereomeric, and epimeric forms as well as mixtures thereof.

In some embodiments, compounds described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrug forms of the herein described compounds, wherein the prodrug is metabolized in vivo to produce a compound of (I) as set forth herein are included within the scope of the claims. Prodrug forms of the herein described compounds, wherein the prodrug is metabolized in vivo to produce a compound of Formula (I), and/or additional active agent, as set forth herein are included within the scope of the claims. In some cases, some of the compounds described herein may be a prodrug for another derivative or active compound. In some embodiments described herein, hydrazones are metabolized in vivo to produce a compound of Formula (I), and/or additional active agent.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

The pharmaceutical compositions described herein, which include a compound of Formula (I), and/or additional active agent, are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

### Certain Systemically Administered Compositions

In one aspect, a compound of Formula (I), and/or additional active agent, is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, compounds described herein are formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a compound of Formula (I), and/or additional active agent, is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

Representative intranasal formulations are described in, for example, U.S. Pat. Nos. 4,476,116, 5,116,817 and 6,391,452. Formulations that include a compound of Formula (I), and/or additional active agent, are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

In some embodiments, pharmaceutical formulations of a compound of Formula (I), and/or additional active agent, are in the form of a capsules, including push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added. A capsule may be prepared, for example, by placing the bulk blend of the formulation of the compound described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration.

In one aspect, solid oral dosage forms are prepared by mixing a compound of Formula (I) with one or more of the following: antioxidants, flavoring agents, and carrier materials such as binders, suspending agents, disintegration agents, filling agents, surfactants, solubilizers, stabilizers, lubricants, wetting agents, and diluents.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder.

Compressed tablets are solid dosage forms prepared by compacting the bulk blend of the formulations described above. In various embodiments, tablets will include one or more flavoring agents.

In other embodiments, the tablets will include a film surrounding the final compressed tablet. In some embodiments, the film coating can provide a delayed release of the compound of Formula (I), and/or additional active agent, from the formulation. In other embodiments, the film coating aids in patient compliance (e.g., Opadry® coatings or sugar coating). Film coatings including Opadry® typically range from about 1% to about 3% of the tablet weight.

In some embodiments, solid dosage forms, e.g., tablets, effervescent tablets, and capsules, are prepared by mixing particles of a compound with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to an insecticide compound the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include a compound of Formula (I), and/or additional active agent, are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, an insecticide compound is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations are common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of a compound of Formula (I), and/or additional active agent, and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a compound of Formula (I), and/or additional active agent, is formulated to include one or more pharmaceutical excipients and flavors. Such a powder is prepared, for example, by mixing the compound and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

### Controlled Release Formulations

In some embodiments, the pharmaceutical dosage forms are formulated to provide a controlled release of a compound of Formula (I), and/or additional active agent. Controlled release refers to the release of the compound from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a compound of Formula (I), and/or additional active agent, that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following:

Shellac—this coating dissolves in media of pH>7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)—PVAP dissolves in pH>5, and it is much less permeable to water vapor and gastric fluids.

Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e. multiparticulate) each containing the formulation described herein. The first group of particles provides a substantially immediate dose of the compound of Formula (I), and/or additional active agent, upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a compound of Formula (I), and/or additional active agent, and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of a compound of Formula (I), the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween ° 60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, glycyrrhiza (licorice) syrup, monoammonium glyrrhizinate (MagnaSweet®), maltol, mannitol, menthol, neohesperidine DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

### Dosages

In some embodiments, a compound of Formula (I) is administered in one or a plurality of doses, each dose comprising from about 1 mg to about 2000 mg, from about 10 mg to about 2000 mg, from about 20 mg to about 2000 mg, from about 30 mg to about 2000 mg, from about 40 mg to about 2000 mg, from about 50 mg to about 2000 mg, from about 60 mg to about 2000 mg, from about 70 mg to about 2000 mg, from about 80 mg to about 2000 mg, from about 90 mg to about 2000 mg, from about 100 mg to about 2000 mg, from about 150 mg to about 2000 mg, from about 200 mg to about 2000 mg, from about 250 mg to about 2000 mg, from about 300 mg to about 2000 mg, from about 350 mg to about 2000 mg, from about 400 mg to about 2000 mg, from about 450 mg to about 2000 mg, from about 500 mg to about 2000 mg, from about 550 mg to about 2000 mg, from about 600 mg to about 2000 mg, from about 650 mg to about 2000 mg, from about 700 mg to about 2000 mg, from about 750 mg to about 2000 mg, from about 800 mg to about 2000 mg, from about 850 mg to about 2000 mg, from about 900 mg to about 2000 mg, from about 950 mg to about 2000 mg, from about 1000 mg to about 2000 mg, from about 50 mg to about 1000 mg, from about 60 mg to about 1000 mg, from about 70 mg to about 1000 mg, from about 80 mg to about 1000 mg, from about 90 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg of a compound of Formula (I). In some cases, the dose comprises from about 50 mg to about 1000 mg, from about 50 mg to about 900 mg, from about 50 mg to about 800 mg, from about 50 mg to about 700 mg, from about 50 mg to about 600 mg, from about 50 mg to about 500 mg, from about 100 mg to about 1000 mg, from about 100 mg to about 900 mg, from about 100 mg to about 800 mg, from about 100 mg to about 700 mg, from about 100 mg to about 600 mg, from about 100 mg to about 500 mg, from about 200 mg to about 1000 mg, from about 200 mg to about 900 mg, from about 200 mg to about 800 mg, from about 200 mg to about 700 mg, from about 200 mg to about 600 mg, from about 200 mg to about 500 mg, from about 300 mg to about 1000 mg, from about 300 mg to about 900 mg, from about 300 mg to about 800 mg, from about 300 mg to about 700 mg, from about 300 mg to about 600 mg, from about 300 mg to about 500 mg, from about 400 mg to about 1000 mg, from about 400 mg to about 900 mg, from about 400 mg to about 800 mg, from about 400 mg to about 700 mg, from about 400 mg to about 600 mg, or from about 400 mg to about 500 mg of a compound of Formula (I). In some cases, the dose comprises about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of a compound of Formula (I).

In some embodiments, a compound of Formula (I) is administered in a single dose. In some embodiments, a compound of Formula (I) is administered in a plurality of doses, e.g., about 2, 3, 4, 5, 6 or 7 doses. In some cases, the compound is not administered in more than 1, 2, 3, 4, 5, 6, or 7 doses. In an exemplary embodiment, the dose is orally administered. If the compound of Formula (I) is administered in a plurality of doses, in some cases, the plurality of doses is administered over a course of less than or equal to about 7, 6, 5, 4, 3, 2 or 1 days. In some cases, the plurality of doses is administered over a course of less than or equal to 3 days.

In some embodiments, a compound of Formula (I) is administered not more than every 1 month, 2 months, 3 months, 4 months, 5 months, 6 months, 7 months, 8 months, 9 months, 10 months, 11 months, 12 months, 1 year, or 2 years. In some cases, the compound of Formula (I) is administered once per season, e.g., a season based on the prevalence of mosquitos, such as a rainy season.

### Certain Topical Compositions

In some embodiments, compounds of Formula (I), and/or additional active agent, are prepared as transdermal dosage forms. In one embodiment, the transdermal formulations described herein include at least three components: (1) a formulation of a compound of Formula (I), and/or additional active agent; (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further includes a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of compounds described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the compounds described herein can be accomplished by means of iontophoretic patches and the like. In one aspect, transdermal patches provide controlled delivery of a compound of Formula (I), and/or additional active agent. In one aspect, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

In alternative embodiments, a compound of Formula (I), and/or additional active agent, is formulated and presented as a wash or rinse liquid which is used to irrigate the affected area. In further embodiments, a compound of Formula (I), and/or additional active agent, is formulated and presented as a spray which is applied to the affected area.

### EXAMPLES

Mosquito colonies utilized in the examples were generally maintained as described: The colony of Anopheles stephensi (Sind-Kasur Nijmegen strain) was maintained at the Radboud University Medical Center, Nijmegen, The Netherlands, by 30° C. and 70-80% humidity and on a 12/12 hour day/night cycle. The Anopheles gambiae strains Kisumu and Tiassalé and the Aedes aegypti strains New Orleans and Cayman were reared at the Liverpool Insect Testing Establishment, Liverpool, UK. Kisumu is an insecticide susceptible lab strain. New Orleans was originally colonized by the Centers for Disease Control and Prevention. The Tiassalé strain was colonized from Southern Cote D'Ivoire where resistance to all classes of insecticide is found and the Cayman strain was colonized from Grand Cayman, where Aedes aegypti are highly resistant to DDT and pyrethroid insecticides. Both resistant strains are routinely selected with insecticides to ensure the maintenance of resistance, 0.75% permethrin for Cayman and 0.05% deltamethrin for Tiassalé, and profiled for resistance to a range of insecticides, including 4% dieldrin, to which Tiassalé is resistant but Cayman is susceptible.

### Example 1. Identification of Mosquitocidal Agents

A high throughput phenotypic screening method was developed to identify new insect vector control agents. The method involves DNA-barcoding to trace individual insects during experiments. To identify novel mosquitocidal agents, DNA-barcoded microspheres were mixed with a bloodmeal and test compound prior to membrane feeding of Anopheles stephensi on 96-well plates. Each well contained a unique barcode and test specimen. Twenty-four hours post feeding, dead mosquitos were pooled and used in a PCR amplification and Luminex-based multiplex detection of barcode sequences to identify compounds with mosquitocidal activity. Similarly, the fraction of live mosquitos was analyzed to assess sampling of every well. Plate feeding was very efficient, with over 90% of fed mosquitos and minimal cross-feeding between different wells. The barcoding approach reliably detected positive control compounds that were spiked in different wells on the plate. Screening of a chemical library identified a number of compounds with potent adulticidal activity against Anopheles. Two of these compounds (isoxazoline compounds fluralaner and afoxolaner) appeared to have excellent pharmacokinetic properties in Beagle dogs and showed plasma levels well above the IC₉₀ for more than eighty days at well-tolerated doses. These compounds are promising candidates for development of mosquitocidal drugs for human or veterinary use.

### Example 2. Activity of Isoxazoline Compounds in Anopheles and Aedes Mosquitos

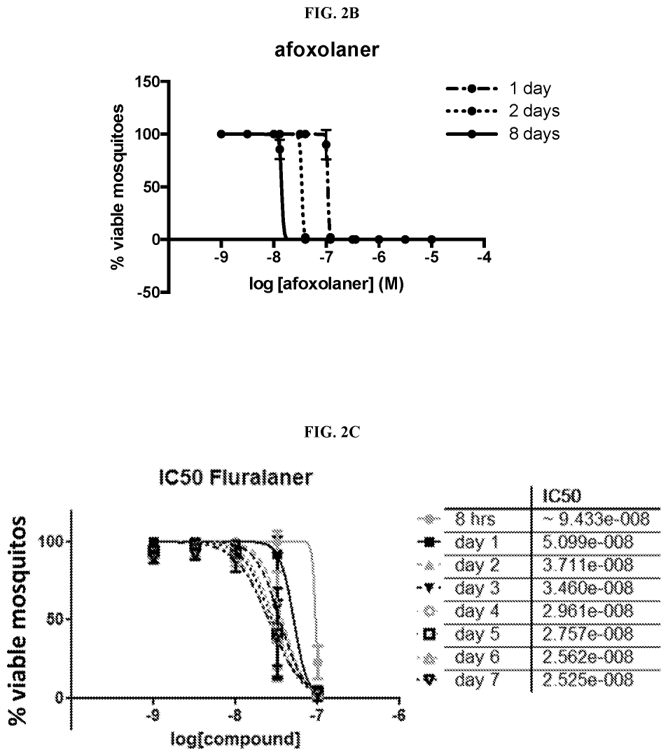

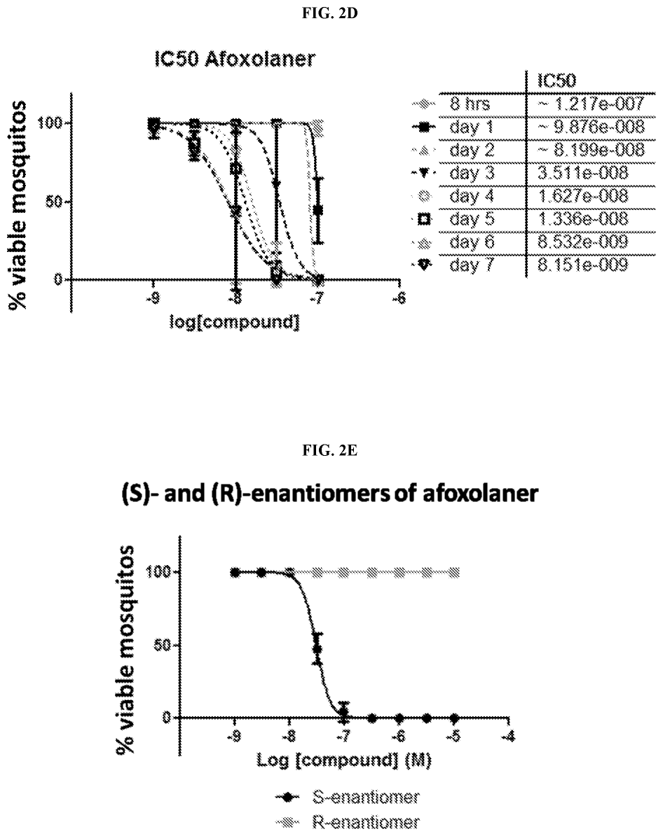

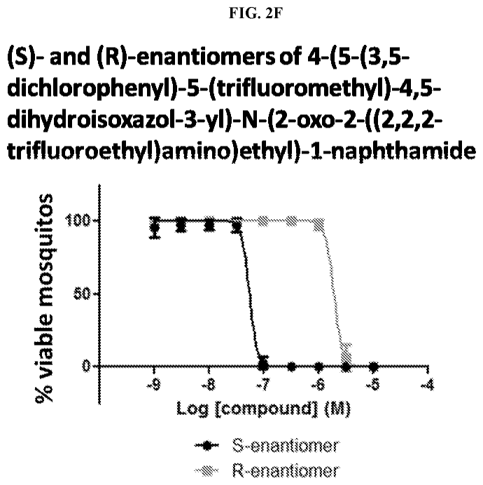

Isoxazoline compounds fluralaner or afoxolaner were reconstituted in 50% human serum and 50% red blood cells and administered to adult Anopheles stephensi mosquitos via standard membrane feeding. Lethality was assessed 1, 2 or 8 days after exposure. FIG. 2A represents a plot of viable Anopheles stephensi versus fluralaner concentration at each day of assessment. FIG. 2B represents a plot of viable Anopheles stephensi versus afoxolaner concentration at each day of assessment. Both figures show that the isoxazoline compounds were effective at killing the mosquitos in a dose-dependent manner. The IC50 values of each compound for Anopheles death were determined by logistic regression using a least squares method to find the best fit and are shown in Table 1. IC50 values were calculated by applying a four-parameter logistic regression model using a least-squares method to find the best fit using the Graphpad Prism 5.0 software package.

TABLE 1
Inhibitory concentration of isoxazoline compounds for Anopheles.

| | IC50 (nM) | | |
|---|---|---|---|
| | 1 day | 2 days | 8 days |
| Fluralaner | 54 | 42 | 37 |
| Afoxolaner | 107 | 34 | 14 |

This experiment was repeated and lethality was assessed 8 hours, and 1, 2, 3, 4, 5, 6, and 7 days after exposure to various concentrations of fluralaner, afoxolaner, (S)- and (R)-enantiomers of afoxolaner, and (S)- and (R)-enantiomers of 4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide. FIG. 2C represents a plot of viable Anopheles stephensi versus fluralaner concentration at each time of assessment. FIG. 2D represents a plot of viable Anopheles stephensi versus afoxolaner concentration at each time of assessment. FIG. 2E represents plots of viable Anopheles stephensi versus concentration of (S)- and (R)-enantiomers of afoxolaner at each time of assessment. FIG. 2F represents plots of viable Anopheles stephensi versus concentration of (S)- and (R)-enantiomers of 4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide at each time of assessment. The IC50 (M) concentrations are also shown for FIGS. 2C and 2D.

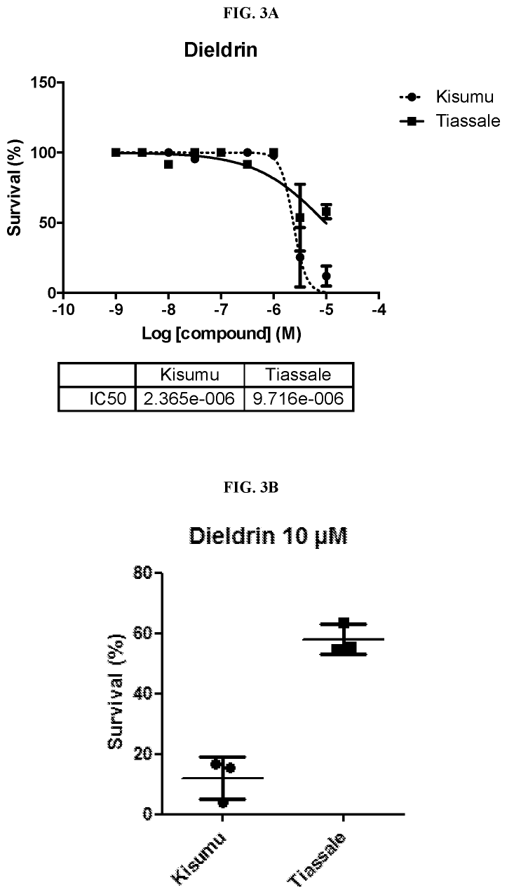

The membrane feeding experiment described was repeated using the insecticide dieldrin on two strains of Anopheles gambiae: Kisumu and Tiassale. The Tiassale strain has a mutation in the resistance to dieldrin (rdl) locus, which codes for γ-aminobutyric acid (GABA) receptor. The Tiassale strain was resistant to pyrethroids, carbamate, DDT and dieldrin (knockdown resistance (kdr), Ace and rdl mutations). The Kisumu strain was susceptible to killing by all tested insecticides. FIG. 3A represents a plot of percentage survival of Kisumu and Tiassale strains of Anopheles gambiae versus dieldrin concentration. FIG. 3B represents a plot of survival of the Kisumu and Tiassale strains at 10 μM dieldrin. As shown in FIG. 3A and FIG. 3B, the Tiassale strain confers resistance to killing by dieldrin. Both figures indicate that the dose-response in systemic feed experiment performed here in allows for identification of a resistance profile.

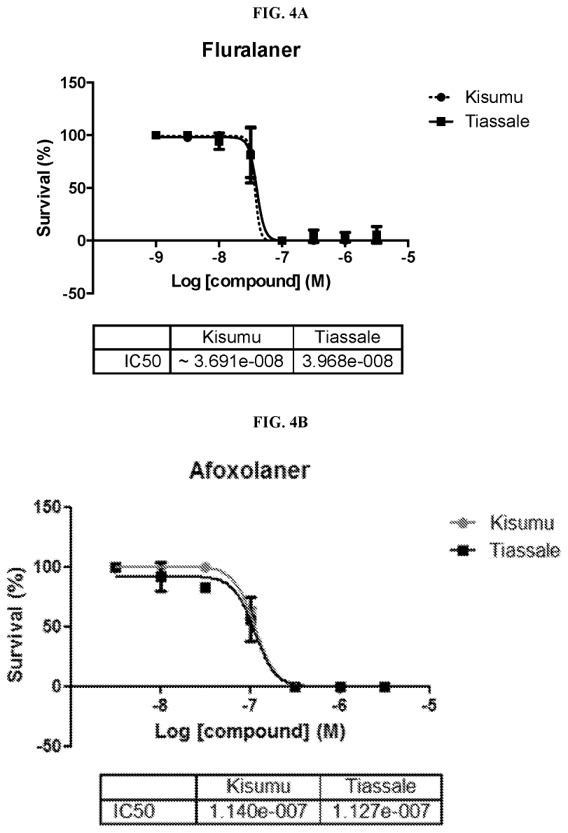

The feed experiment in Anopheles gambiae was repeated using the isoxazoline compounds fluralaner and afoxolaner to determine if the rdl mutation confers resistance to the isoxazoline insecticides. Isoxazoline insecticides target Rdl, but in a location thought to be different from dieldrin. The mosquitos were fed fluralaner or afoxolaner at various concentrations and lethality was assessed after 24 hours. FIG. 4A represents a plot of percentage survival of Kisumu and Tiassale strains of Anopheles gambiae versus fluralaner concentration. FIG. 4B represents a plot of percentage survival of Kisumu and Tiassale strains of Anopheles gambiae versus afoxolaner concentration. Both figures indicate that the rdl mutation in Anopheles gambiae does not lead to resistance to the isoxazoline compounds.

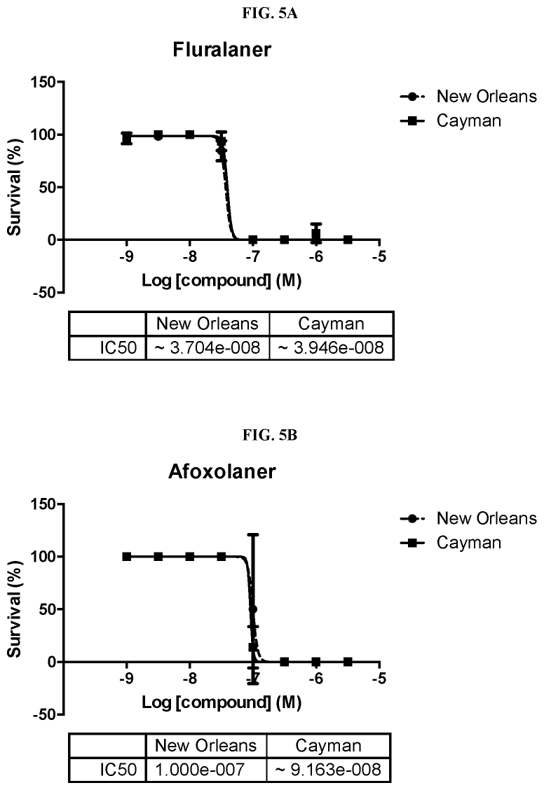

The feed experiment was repeated in Aedes aegypti with the isoxazoline compounds fluralaner and afoxolaner. Two strains of Aedes aegypti were fed as described previously, the New Orleans strain and the Cayman strain. The New Orleans strain was susceptible to all tested insecticides except carbamate (Ace mutation). The Cayman strain was resistant to pyrethroids, carbamate, and DDT (kdr and Ace mutation). No rdl mutation was identified in either colony of the New Orleans or Cayman strains. FIG. 5A represents a plot of percentage survival of New Orleans and Cayman strains of Aedes aegypti versus fluralaner concentration. FIG. 5B represents a plot of percentage survival of New Orleans and Cayman strains of Aedes aegypti versus afoxolaner concentration. Both figures indicate that neither strain of Aedes aegypti were resistant to the isoxazoline compounds tested. The full dose-response shown indicates equipotency of the isoxazoline compounds against Anopheles and Aedes mosquitos.

The IC50 (nM) data is shown in Table 2, with 95% confidence interval (CI) shown in brackets [ ]. Twenty-four hours after the blood meal, fluralaner showed IC50 values in the range of 33 to 56 nM against all strains tested, and afoxolaner showed IC50 values ranging from 90 to 107 nM.

TABLE 2
Inhibitory concentration of isoxazoline compounds for various Anopheles and Aedes mosquitos.

| | IC50 (nM) [95% CI] | | | | |
|---|---|---|---|---|---|
| | | Anopheles | Anopheles | | |
| | Anopheles | gambiae | gambiae | Aedes aegypti | Aedes aegypti |
| | stephensi | (Kisumu) | (Tissalé) | (New Orleans) | (Cayman) |
| Fluralaner | 55.99 [55-57] | 33.31 [33-34] | 33.16 [32-34] | 34.18 [33-35] | 35.80 [34-37] |
| Afoxolaner | 106.8 [102-118] | 101.4 [100-103] | 100.7 [99-102] | 100.0 [93-107] | 90.23 [87-94] |

Without being bound by theory, isoxazolines occupy a binding site that is distinct from the targets of known modulators of ionotropic GABA receptors. In line with this notion, fluralaner and afoxolaner were fully active against the Anopheles gambiae Tiassalé strain that carries the resistance-to-dieldrin (rdl) mutation in the GABA receptor. In addition, they were equipotent against pyrethroid- and carbamate-resistant strains carrying mutations in the kdr sodium channel and acetylcholine esterase (Ace-1) genes respectively. Table 3 shows characterization of the insecticide resistance in different strains of Anopheles and Aedes mosquitoes housed at the Liverpool Insect Testing Establishment using the standard WHO paper contact assay following exposure to the drugs indicated in the table (test kits and insecticide impregnated papers supplied by Universiti Sains Malaysia (USM), Penang). The results are shown as percentage of survival (on over 100 mosquitoes for each case), the bold and underlined numbers indicate values that fall within the criteria for resistance.

TABLE 3
Characterization of insecticide resistance in different strains of Anopheles and Aedes mosquitos.

| | Target | Sodium channel | | | Acetylcholine esterase | | GABACL |
|---|---|---|---|---|---|---|---|
| | Mutation | kdr | | | Ace | | rdl |
| | type | | | | | | |
| | Family | pyrethroid | | | carbamate | organo- | |
| | | | | | | phosphate | |
| | Molecule | 0.75% | 0.05% | 4% DDT | 0.1% | 1.0% | 4% |
| | | Permethrin | Delta- | | Bendiocarb | Fenitrothion | Dieldrin |
| | | | methrin | | | | |
| Anopheles | Kisumu | 100 | 100 | 100 | 100 | 100 | 96 |
| gambiae | Tiassale 13 | 3 | 10 | 3 | 11 | 96 | 21 |
| Aedes | New | 100 | 100 | 100 | 27 | 100 | 100 |
| aegypti | Orleans | | | | | | |
| | Cayman | 7 | 36 | 0 | 20 | 100 | 95 |

The membrane feeding experiment was repeated in Anopheles with test compounds afoxolaner racemate, (S)-afoxolaner, and (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide. At day 7, the potency of each compound was evaluated by calculating the EC₉₀ concentrations, as shown in Table 4.

TABLE 4
Effective concentrations of isoxazoline compounds in Anopheles
membrane feeding assay.

| | EC₉₀ (nM) |
|---|---|
| afoxolaner racemate | 8.1 |
| (S)-afoxolaner | 8.0 |
| (S)-4-(5-(3,5-dichlorophenyl)-5-(trifluoromethyl)-4,5- | 8.0 |
| dihydroisoxazol-3-yl)-N-(2-oxo-2-((2,2,2- | |
| trifluoroethyl)amino)ethyl)-1-naphthamide | |

### Example 3. Modeling the Mosquitocidal Impact of an Isoxazoline Compound in a MDA

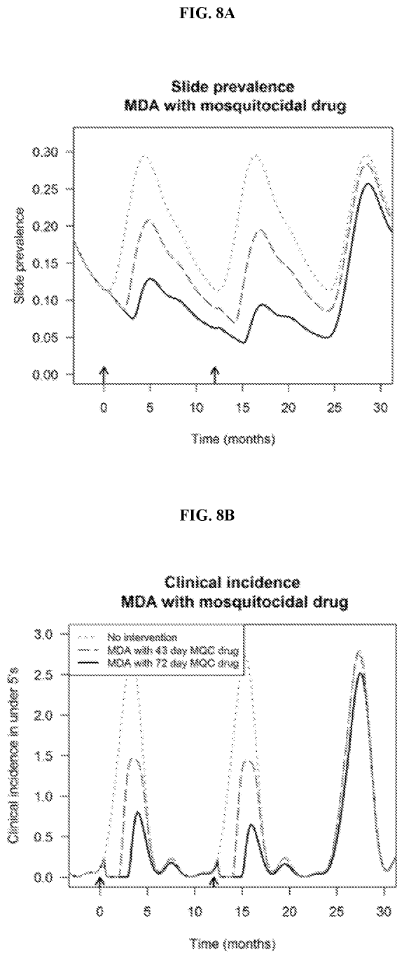

A first model of a mass drug administration (MDA) scenario was designed with an isoxazoline for a 43 or 72 day mosquitocidal duration in a seasonal transmission setting. The modeling algorithm was derived from Slater et al., The Journal of Infectious Diseases, 210: 1972-1980. In this model, the compound is given to 80% of the population (over the age of 5) once at the start of the rainy season. This administration is repeated for 2 years. The impact of isoxazoline versus no intervention on prevalence by microscopy as modeled is shown in FIG. 8A. The impact of isoxazoline versus no intervention on clinical incidence as modeled is shown in FIG. 8B. For the isoxazoline compound with a 43 day effective duration, there is a calculated 52% reduction in clinical cases as compared to no intervention. For the isoxazoline compound with a 72 day effective duration, there is a calculated 81% reduction in clinical cases as compared to no intervention.

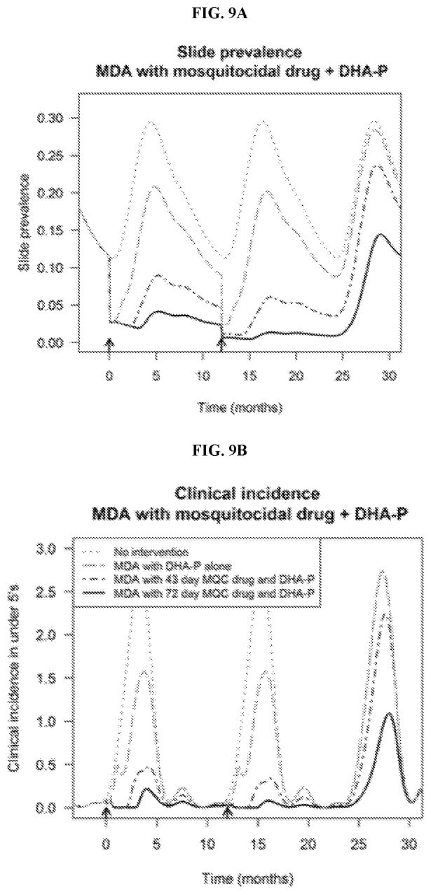

A second model of a mass drug administration (MDA) scenario was designed with an isoxazoline and dihydroartemisinin-piperaquine (DHA-P) for a 43 or 72 day effective mosquitocidal duration in a seasonal transmission setting. The compounds are given to 80% of the population (over the age of 5) once at the start of the rainy season. This administration is repeated for 2 years. The impact of isoxazoline and DHA-P versus no intervention on prevalence by microscopy as modeled is shown in FIG. 9A. The impact of isoxazoline and DHA-P versus no intervention on clinical incidence as modeled is shown in FIG. 9B. For the isoxazoline compound with a 43 day effective duration in combination with DHA-P, there is a calculated 84% reduction in clinical cases as compared to no intervention. For the isoxazoline compound with a 72 day effective duration in combination with DHA-P, there is a calculated 95% reduction in clinical cases as compared to no intervention. For the 43 day duration isoxazoline compound with DHA-P, there is a calculated additional 75% reduction in clinical cases as compared to DHA-P alone. For the 72 day duration isoxazoline compound with DHA-P, there is a calculated additional 92% reduction in clinical cases as compared to DHA-P alone.

### Example 4. Mass Drug Administration of an Isoxazoline Compound

MDA studies with ivermectin have shown about an 80% reduction in infectious Anopheles gambiae and an estimated 16% reduction in new malaria cases in children under 5 (Trends Parasitol. 2011 October; 27(10): 423-428). Co-treatment of ivermectin and artemether-lumefantrine have shown an estimated 35% reduction in transmission potential, though the effect was short-lived (Clin Infect Dis. (2015) 60 (3): 357-365). One method being pursued to overcome the transient nature of these effects is to produce long-acting formulations of ivermectin, for example, intramuscular depot injections. A simpler solution that is likely to be lower cost and meet with better compliance is to provide an oral isoxazoline compound. A model was produced that indicates the potential for an 80% reduction in clinical malaria cases when an isoxazoline compound is used as a standalone drug in MDA, or a 95% reduction when the isoxazoline compound is used in combination with DHA-piperaquine. Table 5 provides exemplary regimens of MDA programs.

TABLE 5
Mass drug administration of afoxolaner.

| | Impact on | Impact on |
|---|---|---|
| | Clinical Cases** | Clinical Cases** (w/ |
| | (alone) | DHA-P) |
| Regimen (MDA to 80% of | Relative to no | Relative to |
| population) | intervention | DHA-P alone |
| Afoxalaner (4 mg/kg/day for one | 81% reduction | 92% reduction |
| day) | | |
| Ivermectin (0.15 mg/kg/day for | 11% reduction | 26% reduction |
| three days) | | |
| Ivermectin (0.60 mg/kg/day for | ~20% reduction | ~50% reduction |
| three days) | | |

**Assumes a fairly high transmission setting: 25% prevalence by microscopy and 50% by PCR

### Example 5. Cytochrome Inhibition

Reversible inhibition of cytochrome P450s (CYPs) in human liver microsomes was tested using prototypical, isoform-selective activity assays for individual CYPs in a pool of human liver microsomes (i.e., testosterone for CYP 3A4, phenacetin for CYP 1A2, bufuralol for CYP 2D6, S-mephenytoin for CYP 2C19, and diclofenac for CYP 2C9). The assay provides 8-point IC₅₀ determinations in the range of 0.010 to 50 μM drug concentrations for major isoforms of CYP450. These assays were used to evaluate the activity of isoxazoline compounds, as shown in Table 6.

TABLE 6
Cytochrome inhibition assay.

| | | | (S)-4-(5-(3,5- |
|---|---|---|---|
| | | | dichlorophenyl)-5- |
| | | | (trifluoromethyl)-4,5- |
| | | | dihydroisoxazol-3-yl)-N-(2- |
| | | | oxo-2-((2,2,2- |
| | | | trifluoroethyl)amino)ethyl)- |
| | afoxolaner racemate | (S)-afoxolaner | 1-naphthamide |
| Cyp microsome (5 | >50 uM (3A4, 1A2, 2D6); | >50 uM (3A4, 1A2); | >50 uM (3A4, 1A2); 2C19 = |
| isoforms) IC₅₀ | 2C19 = 3.1 uM; | 2C19 = 1.1 uM; | 0.6 uM; |
| | 2C9 = 2.9 uM | 2C9 = 4.2 uM; | 2C9 = 1.9 uM; |
| | | 2D6 = 19 | 2D6 = 17.2 |

### Example 6. Tissue Binding Assay of Isoxazoline Compounds

The percentages of compounds bound to brain homogenate were evaluated for (S)-afoxolaner and (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide, as shown in Table 7. Afoxolaner racemate was not determined (ND).

Briefly, frozen tissue homogenate (homogenized with buffer at a certain volume ratio) was used as a test matrix. The tissue homogenate was purchased from Bioreclamation (Hicksville, N.Y., USA) or prepared by WuXi AppTec.

Isoxazoline compounds were spiked into blank tissue homogenate at the test concentration. Triplicate samples were used.

An appropriate volume of spiked tissue sample was removed before incubation for recovery calculation. An aliquot (e.g., 150 μL) of matrix sample was added to the donor side of the chamber (donor chamber) in a dialyzer plate (HTD dialysis device or RED device) and a certain amount of dialysis buffer was loaded to the other side of the chamber (receiver chamber). Triplicate incubations were performed (or other replicate number according to specific requirement).

The dialyzer plate was placed into a humidified incubator with 5% CO2 and incubated at 37° C. for appointed time (the general incubation time is 4-6 h).

After incubation, samples were removed from the donor side as well as the receiver side.

The samples were matched with appropriate amount of the opposite blank matrix (tissue homogenate or buffer).

The matrix-matched samples were quenched with stop solution containing internal standard (IS).

The samples were analyzed by LC-MS/MS. Test compound concentrations in matrix and buffer samples were assessed based on the peak area ratios (PAR) of analyte/internal standard (no standard curve).

TABLE 7
Brain homogenate binding assay

| | | | (S)-4-(5-(3,5- |
|---|---|---|---|
| | | | dichlorophenyl)-5- |
| | | | (trifluoromethyl)-4,5- |
| | | | dihydroisoxazol-3-yl)-N-(2- |
| | | | oxo-2-((2,2,2- |
| | afoxolaner | | trifluoroethyl)amino)ethyl)- |
| | racemate | (S)-afoxolaner | 1-naphthamide |
| Brain | ND | >99.9% | >99.9% |
| homogenate | | | |
| % binding | | | |

### Example 7. In Vitro Metabolic Stability of Isoxazoline Compounds in cd-1 Mouse, sd Rat, Beagle Dog, Cynomolgus Monkey and Human Cryopreserved Hepatocytes

Isoxazoline compounds (at 1 μM) were incubated with cryopreserved hepatocytes (0.5×10⁶ cells per mL) in duplicates (n=2) at 37° C. using 96-well plate format.

Time points were 0, 15, 30, 60 and 90 minutes in separate plates and medium control samples without cells at 0 and 90 minutes were also incubated. At each time point the reaction was stopped by adding organic solution containing internal standard (IS).

Positive controls 7-ethoxycoumarin and 7-hydroxycoumarin were included in parallel.

The samples were analyzed by LC-MS/MS. Disappearance of the test compound was assessed based on peak area ratios of analyte/IS (no standard curve).

Intrinsic clearance and t½ values were then calculated.

The half-life of afoxolaner racemate, (S)-afoxolaner, and (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide in hepatocytes from mouse (ms), rat (r), dog (d), cynomolgus monkey (cy), and human (hu) is shown in Table 8.

TABLE 8
Hepatocyte stability assay

| | | | (S)-4-(5-(3,5- |
|---|---|---|---|
| | | | dichlorophenyl)-5- |
| | | | (trifluoromethyl)-4,5- |
| | | | dihydroisoxazol-3-yl)-N-(2- |
| | | | oxo-2-((2,2,2- |
| | afoxolaner | | trifluoroethyl)amino)ethyl)- |
| | racemate | (S)-afoxolaner | 1-naphthamide |
| Hepatocyte | >217 (ms), | 64 (ms), | 59 (ms), |
| stability | >217 (r), | 60 (r), | 77 (r), |
| (t½) (min) | >217 (d), | 32 (d), | 41 (d), |
| | >217 (cy), | 50 (cy), | 42 (cy), |
| | >217 (hu) | 66 (hu) | 63 (hu) |

In a further assay, isoxazolines (fluralaner and afoxolaner) and control compounds (7-Ethoxycoumarin and 7-Hydroxycoumarin [Sigma]) were dissolved in DMSO at 10 and 30 mM respectively, then diluted first 20-fold with 45% methanol in water and a further 10-fold in pre-warmed Williams' Medium E. Cryopreserved human, dog and rat hepatocytes (In Vitro Technologies) were thawed, isolated by percoll gradient and suspended in Williams' Medium E. Cells were then dispensed into the wells of 96-well plates containing 10 μL of diluted compounds, to reach a final concentration of 0.5×10⁶ cells/mL and 1 μM of isoxazolines or 3 μM of control. After an incubation at 37° C. of 0, 15, 30, 60 or 90 minutes, the reaction was stopped with acetonitrile. The samples were then shaken for 10 minutes at 500 rpm and centrifuged at 3220 g for 20 minutes. Supernatants were transferred and stored at 4° C. until LC-MS-MS analysis. The results are shown in Table 9. Neither fluralaner nor afoxolaner showed measurable metabolization when incubated with human hepatocytes, suggesting the low intrinsic clearance observed in dogs would translate to humans.

TABLE 9
In vitro degradation of fluralaner, afoxolaner, ethoxycoumarin and
hydroxycoumarin by human, dog and rat primary hepatocytes.
The results are shown as half-life of each compound (t1/2) and in vitro
clearance (Cl int). The results are based on 5-point time courses.

| | Human hepatocytes | | Dog hepatocytes | | Rat hepatocytes | |
|---|---|---|---|---|---|---|
| | | Cl int | | Cl int | | Cl int |
| | | (μL/min/10\hat{ }6 | | (μL/min/10\hat{ }6 | | (μL/min/10\hat{ }6 |
| | t1/2 (min) | cells) | t1/2 (min) | cells) | t1/2 (min) | cells) |
| Fluralaner | >216.8 | <6.4 | >216.8 | <6.4 | >216.8 | <6.4 |
| Afoxolaner | >216.8 | <6.4 | >216.8 | <6.4 | >216.8 | <6.4 |
| 7- | 20.1 | 69.1 | 14.0 | 99.3 | 61.8 | 22.4 |
| Ethoxycoumarin | | | | | | |
| 7- | 14.7 | 94.1 | 20.4 | 68.1 | 4.8 | 290.5 |
| Hydroxycoumarin | | | | | | |

### Example 8. Plasma Protein Binding Assay with Isoxazoline Compounds

Frozen plasma (EDTA-K2 as anticoagulant) pooled from multiple individuals of various species was used as a test matrix. The plasma was purchased from commercial vendors or prepared in house from animals. Warfarin was used as a positive control.

Isoxazoline compounds were spiked into the blank matrix at the final concentration of 2 μM.

A 150 μL aliquot of matrix sample was added to one side of the chamber in a 96-well equilibrium dialyzer plate (HTD dialysis) and an equal volume of dialysis buffer was added to the other side of the chamber. An aliquot of matrix sample was harvested before the incubation and used as T₀ samples for recovery calculation. Triplicate incubations were performed.

The dialyzer plate was placed in a humidified incubator and rotated slowly for 4 hours at 37° C. After incubation, samples were taken from the matrix side as well as the buffer side. The plasma sample was matched with equal volume of blank buffer; and buffer samples were matched with equal volume of blank plasma. The matrix-matched samples were quenched with a stop solution containing internal standard.

The samples were analyzed by LC-MS/MS. The concentrations of isoxazoline compounds in matrix and buffer samples were expressed as peak area ratios (PAR) of analyte/internal standard (no standard curve).

Then, % bound fraction at 4 hours (% bound=1−% unbound) and % recovery in incubates at 4 hours were calculated.

The percentages of compounds bound to mouse (ms), rat (r), dog (d), and human (hu) plasma are shown in Table 7. The stability of compounds in mouse (ms), rat (r), dog (d), cynomolgus monkey (cy), and human (hu) plasma is shown in Table 10 by the percentages of compounds remaining at two hours. Plasma stability at two hours was not determined (ND) for (S)-afoxolaner and (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide.

TABLE 10
Plasma protein binding and stability

| | | | (S)-4-(5-(3,5- |
|---|---|---|---|
| | | | dichlorophenyl)-5- |
| | | | (trifluoromethyl)-4,5- |
| | | | dihydroisoxazol-3-yl)-N-(2- |
| | | | oxo-2-((2,2,2- |
| | | | trifluoroethyl)amino)ethyl)- |
| | afoxolaner racemate | (S)-afoxolaner | 1-naphthamide |
| Plasma protein % binding | 97.8 (ms), | >99.9 (ms, r, hu), | >99.9% (ms, r, d, hu) |
| | 97.3 (r), | 99.5 (d) | |
| | >99.9 (d, hu) | | |
| Plasma stability % remaining | 83% (ms); | ND | ND |
| @ 2 h | 61% (r); | | |
| | 100% (d); | | |
| | 84% (cy); | | |
| | 84% (hu) | | |

### Example 9. hERG Channel In Vitro Patch Clamp Assay of Isoxazoline Compounds

Manual patch clamp assays were performed to determine inhibitory concentrations of afoxolaner racemate, (S)-afoxolaner, and (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide against hERG.

Cells: Stable CHO-K1 cells expressing hERG channels (from AVIVA Biosciences) were used in the assay.

Patch Clamp recording: The recordings were performed on conventional patch clamp (Axon Multiclamp 700B, Digidata 1440, pCLAMP 10, Molecular Devices Corporation or HEKA EPC10/Patchmaster system, HEKA Elektronik Corporation) at room temperature, using the whole-cell patch clamp techniques. The composition of external solution was (mM): HEPES 10, NaCl 145, KCl 4, CaCl2 2, MgCl2 1, Glucose 10, pH to 7.4 with 1N NaOH, osmolarity to 290-300 mOsm. Filtered and kept at 4° C. The composition of internal solution was (mM): KOH 31.25, KCl 120, CaCl2 5.374, MgCl2 1.75, EGTA 10, HEPES 10, Na2-ATP 4, pH to 7.2 with 1N KOH, osmolarity to 280-290 mOsm. Filtered and kept at −20° C.

For conventional patch clamp, 35 mm culture dishes or recording chambers containing CHO-hERG cells were placed on the stage of an inverted/upright microscope and continuously perfused with external solutions from a perfusion system (RSC-160 Rapid solution Changer, BioLogic or OctaFlow I, ALA Scientific Instruments Inc). Micropipettes were filled with the internal solutions and had a resistance of 2-5 MΩ.

Test compounds were dissolved in 100% DMSO to make stock solutions for each test concentration, and then diluted into external solutions to achieve final concentration for testing. Final DMSO concentration was not more than 0.3%.

Voltage command protocol: From the holding potential of −80 mV, the voltage was first stepped to 60 mV for 850 ms to open hERG channels. Then the voltage was stepped back down to −50 mV for 1275 ms, causing a “rebound” or tail current, which was measured and collected for data analysis. Finally, the voltage was stepped back to the holding potential (−80 mV). This voltage command protocol was repeated every 15000 msec. This command protocol was performed continuously during the test (vehicle control, test compounds).

Compound effect (% inhibition) was determined by difference in current amplitude before and after the application of test compounds. IC50 values were determined from concentration-response curves that were obtained with Hill fitting.

Results from the manual patch clamp assay are shown in Table 11.

TABLE 11
hERG channel assay

| | | | (S)-4-(5-(3,5- |
|---|---|---|---|
| | | | dichlorophenyl)-5- |
| | | | (trifluoromethyl)-4,5- |
| | | | dihydroisoxazol-3-yl)-N-(2- |
| | | | oxo-2-((2,2,2- |
| | afoxolaner | | trifluoroethyl)amino)ethyl)- |
| | racemate | (S)-afoxolaner | 1-naphthamide |
| Ion channels | 0.9% ± 2.5% | >30 | 19.9 |
| IC50 | inhibition | | |
| (Manual | at 5 uM | | |
| patch) - | | | |
| hERG | | | |

### Example 10. Bidirectional Permeability in Caco-2 Cells

The suitability of the compound for oral dosing was evaluated via a Caco-2 permeability assay to predict human intestinal permeability and to investigate drug efflux. Afoxolaner racemate was evaluated for P-glycoprotein (PGP) inhibition in a MDCK permeability assay. The permeability data is shown in Table 12.

Caco-2 Cells (obtained from ATCC) were seeded onto PET membranes of 96-well Insert Plates for 21-28 days for confluent cell monolayer formation. The integrity of the monolayer was verified by performing Lucifer yellow rejection assay. The quality of the monolayer was also verified by measuring the unidirectional (A→B) permeability of fenoterol/nadolol (low permeability marker), propranolol/metoprolol (high permeability marker) and Bi-directional permeability of Digoxin (a P-glycoprotein substrate marker) in duplicate wells.

Standard assay conditions are as follows: each test compound was tested at 2 μM (DMSO≤1%) with two replicates; bi-directional transport was evaluated (A→B and B→A); incubation was for 2 hours with a single time point; the transport buffer contained HBSS with 10 mM HEPES at pH7.40±0.05; and incubation occurred at 37±1° C., 5% CO2 and relatively saturated humidity. The dosing solution was spiked and mixed with transport buffer and Stop Solution (containing an appropriate internal standard (IS)) to provide the T0 sample. At the end of incubation, sample solutions from both donor and receiver wells were immediately mixed with Stop Solution. All samples, including T0 samples, donor samples, and receiver samples, were analyzed using LC/MS/MS. Concentrations of test compound are expressed as peak area ratio of analytes versus IS without a standard curve.

TABLE 12
Cell permeability

| | | | (S)-4-(5-(3,5- |
|---|---|---|---|
| | | | dichlorophenyl)-5- |
| | | | (trifluoromethyl)-4,5- |
| | | | dihydroisoxazol-3-yl)-N-(2- |
| | | | oxo-2-((2,2,2- |
| | afoxolaner | | trifluoroethyl)amino)ethyl)- |
| | racemate | (S)-afoxolaner | 1-naphthamide |
| Caco2 A-B/ | <0.0003/ | <0.20/0.7 | <0.29/0.56 |
| B-A | 0.24 | | |
| (10⁻⁶ cm/sec) | | | |
| PGP | >50 | ND | ND |
| inhibition in | | | |
| MDCK | | | |
| (mM) | | | |

### Example 11. Further Characterization of Isoxazoline Compounds

Isoxazoline compounds: afoxolaner racemate, (S)-afoxolaner, and (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide were further profiled using standard compound profiling assays.

The lipophilicities of isoxazoline compounds were evaluated at pH 7.4, with A Log D values shown in Table 13.

The solubility of each compound was determined at pH 6.8, as shown in Table 13.

A study was performed to evaluate pharmacokinetics of a single oral dose of afoxolaner racemate in dog, as shown in Table 13.

The profiling data described in this and previous examples provides for calculation of a projected single oral dose of afoxolaner racemate in humans that would allow for 90 day coverage. As an example, a human is orally administered 450 mg of afoxolaner racemate and the afoxolaner racemate is effective in killing a vector exposed to the afoxolaner racemate during a bite or blood meal with the human, for up to and including 90 days after drug administration.

TABLE 13
Isoxazoline profiling data.

| | | | (S)-4-(5-(3,5- |
|---|---|---|---|
| | | | dichlorophenyl)-5- |
| | | | (trifluoromethyl)-4,5- |
| | | | dihydroisoxazol-3-yl)-N-(2- |
| | | | oxo-2-((2,2,2- |
| | | | trifluoroethyl)amino)ethyl)- |
| | afoxolaner racemate | (S)-afoxolaner | 1-naphthamide |
| ALogD (pH 7.4) | 6.1 | 6.1 | 5.8 |
| Solubility (pH 6.8) (μM) | <1 | <1 | <1 |
| Dog PK (PO) | ~600 nM at Day 45 | ND | ND |
| Projected human dose (90 | ~450 mg | ND | ND |
| day coverage with single | | | |
| PO dose) | | | |

### Example 12. Model of Fluralaner and Afoxolaner in Humans

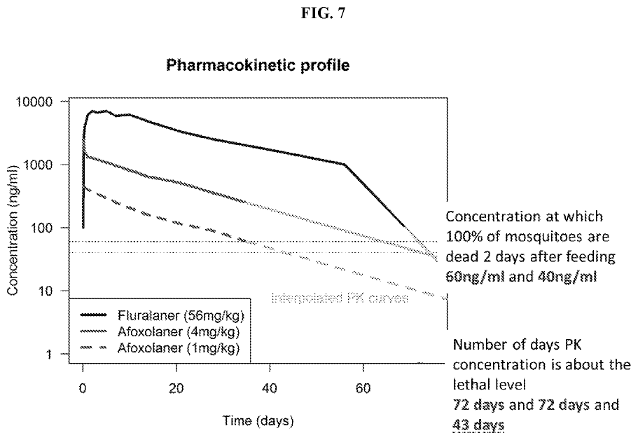

The lethal duration of fluralaner and afoxolaner was estimated by extrapolating pharmacokinetic curves from published studies in Beagle dogs to the killing concentrations determined by the membrane feeding studies described in Example 2. Data for fluralaner in Beagle dogs was obtained from Walther et al., Parasites & Vectors 8:508. Data from afoxolaner in Beagle dogs was obtained from Letendre et al., Veterinary Parasitology, 201, 190-197. A mathematical model was used to estimate the impact of these compounds on Anopheles stephensi mosquito survival. FIG. 7 shows the modeled pharmacokinetic curves. This model indicates that at a dose of 56 mg/kg, the concentration of fluralaner is above the level that is lethal to Anopheles mosquitos for 72 days after feeding, and the concentration of afoxolaner is above lethal level for 43 days (1 mg/kg) or 72 days (4 mg/kg) after feeding.

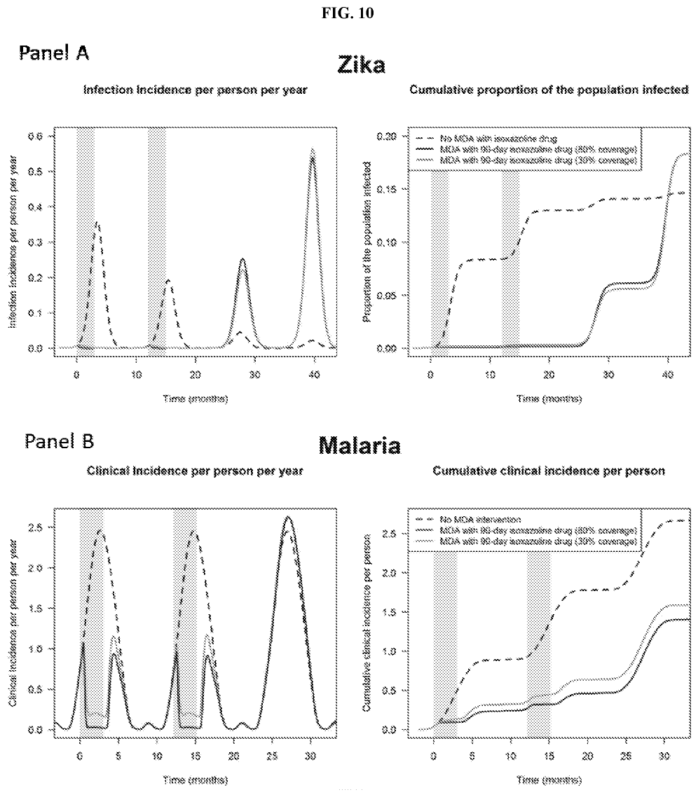

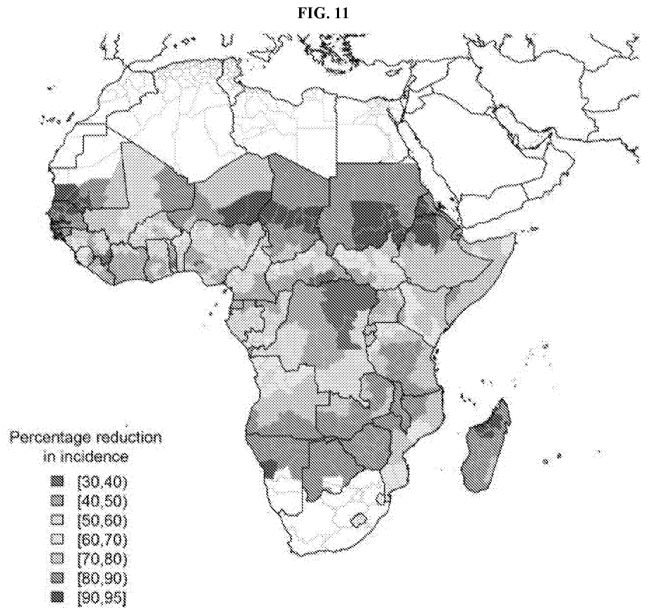

It was estimated that a single total human dose of 450 mg and 750 mg for afoxolaner and fluralaner, respectively, to a 70 kg subject, will result in circulating drug concentrations above the mosquitocidal IC99 for 90 days (Table 14). Since these doses are reasonable amounts to be formulated and delivered in a single-dose mass drug administration, this 90-day period of efficacy was used to model the potential effect on two mosquito-borne diseases. Zika is an immunizing infection, meaning that once an individual has been infected, they are no longer susceptible to new infections. The effect was modeled of an intervention in a population with historical exposure to Zika at a moment in time where herd immunity had declined to a degree that permits a new epidemic. Administration of an isoxazoline drug once a year is predicted to prevent nearly all clinical cases during the years of administration, even if only 30% of the population is being treated (FIG. 10). As soon as the intervention ceases, transmission restarts. This rebound, explained by a reduction in the herd immunity and an increase in the susceptible population (due to births) during the years of intervention, may result in the total number of cases over the 3 years being higher in treated than in untreated populations. Thus, mass drug administration of a mosquitocidal drug may be a very efficient in delaying Zika transmission in populations but may need to be repeated to sustainably prevent outbreaks.

Modeling of malaria incidence: An existing transmission model describing the impact of another mosquitocidal drug, ivermectin, on malaria was extended to simulate the impact of afoxolaner or fluralaner with a 90-day efficacy period, with a 2-year intervention scheme (1 intervention per year at the beginning of the transmission period). The model assumes that mosquitoes taking a bloodmeal containing either drug on any day during the 90 day efficacy window will experience reduced survival—mean lifespan is reduced from 7.6 days pre-intervention to 2 days during the intervention. This translates in less than 1% of the mosquitoes being able to survive until complete sporogony. It was assumed that a proportion (c) of the human population over the age of 5 are treated, and only bloodmeals taken on these individuals results in reduced survivorship. The infectious state of the mosquito (susceptible, latently infected or infectious) was tracked to link the increase in vector mortality to a reduction in the infectious vector density and thus the rate of infections in humans. The impact of afoxolaner/fluralaner was estimated in all malaria endemic areas in Africa. Each 1″ administrative unit (top-level regional divisions) has a specific transmission intensity (based on a prevalence-incidence relationship in the Imperial College malaria model and the Malaria Atlas Project estimates for 2015 prevalence) and seasonality profile based on rainfall data. Mass drug administration (MDA) with a mosquitocidal drug efficacious for 90 days and a coverage of 80% of the population over the age of five was simulated in each 1^{st} administrative unit. The MDA was conducted at an optimal time based on the specific seasonality profile of each administrative unit. The map presented in FIG. 11 is a simplified and illustrative approach to estimating the true impact of this intervention across Africa—in some cases a far wider range of complexities would need to be considered, such as the vector species in each location (currently assumed to be all Anopheles gambiae-like), movement of individuals between locations, each individual country's national malaria strategy (in terms of planned increases in current interventions such as LLIN distribution and access to treatment) and true achievable coverage and compliance in each area.

Modeling of Zika incidence: An existing Zika transmission model was adapted to include an increased rate of vector mortality during the 90 day efficacy period of the drug of 0.5cp K/day where 0.5 translates into a mean lifespan of 2 days for mosquitoes biting a treated subject, c represents drug coverage in individuals over 5 years of age, p is the proportion of the population over 5 years of age (=0.908 for the demography assumed) and K is the biting rate per adult female mosquito (=0.5/day). Treatment occurring in one of twenty spatially coupled geographic regions (parameterized to represent Latin America) was simulated in a population with historical prior exposure to Zika but at a point in time where herd immunity has declined to the point where a new epidemic is able to occur. Treatment started within 2 months of the start of the new epidemic and is repeated exactly one year later. The annualized incidence of infection, and the cumulative infection incidence since the start of the epidemic is tracked.

Total plasma clearance and volume of distribution measured in dog have been scaled to human using single species allometry with typical exponents of 0.75 for clearance and 1.0 for volume assuming a bodyweight of 11 kg for dog and 70 kg for human. As the predicted clearance in man and the measured clearance in dog is much lower than hepatic blood flow, negligible first pass extraction by the liver is expected and oral bioavailability will be a function of absorption. Considering that the reported bioavailability for fluralaner was moderate-low in dogs and non-linear with dose, a bioavailability of 25% was assumed in man for this compound. By contrast, the bioavailability of afoxolaner in dog being relatively high and dose-proportional, the same value of 74% was used for man. Predictions of exposure in man have been made using these estimated parameters (reported in Table 14) and a single compartment PK model assuming first order rate of absorption at a rate of 1 h⁻¹. The doses reported herein are predicted to achieve a total plasma concentration above the whole blood _{IC99} (233 nM=146 ng/mL for afoxolaner and 119 nM=66 ng/mL for fluralaner) for 90 days following a single administration.

TABLE 14
Estimation of pharmacokinetics parameters in human based on the
published values found in dog. CL stands for plasma clearance and
V for volume of distribution.

| | | Dog | Predicted human |
|---|---|---|---|
| | | pharmacokinetics | pharmacokinetics |
| Fluralaner | Oral bioavailability | 11-34% | 25% |
| | CL (mL/min/kg) | 0.0972 | 0.0612 |
| | V (L/kg) | 3.1 | 3.1 |
| | Half-life (days_) | 15.0 | 24.4 |
| Afoxolaner | Oral bioavailability | 74% | 74% |
| | CL (mL/min/kg) | 0.0825 | 0.0519 |
| | V (L/kg) | 2.68 | 2.68 |
| | Half-life (days_) | 15.5 | 24.4 |

Naturally acquired immunity to malaria is mainly non-sterilizing but reduces the severity of infections, and any infectious mosquito bite could potentially cause a new infection. Therefore a transient intervention such as afoxolaner/fluralaner administration would result in a temporary reduction in malaria incidence and a large reduction in cumulative incidence over a specified time period. For example, in a transmission setting with 17% malaria parasite prevalence by microscopy and a short transmission season (−5-6 months), 80% coverage of the population with the drug would result in a 74% reduction in malaria cases in the intervention year. In the same conditions, 64% reduction in cases is achieved with population coverage of only 30%. To further study the impact of prevalence and seasonality, the reduction in clinical malaria cases for the malaria endemic areas of sub-Saharan Africa was estimated based on previously used parameterization of malaria transmission heterogeneity. The results show that the intervention has the highest impact (>80% reduction in clinical cases) in areas with low and very seasonal transmission, such as Senegal, Sudan, Ethiopia, Madagascar, Namibia, Botswana and Zimbabwe (FIG. 11). In the part of the continent with higher transmission and perennial transmission, isoxazoline administration is predicted to lead to a reduction in the number of clinical cases of at least 30 percent. From this simplified and illustrative approach, a single-dose intervention is predicted to have a significant impact on malaria transmission with the greatest efficacy in areas with low transmission and a short transmission season. In some cases, in areas with longer rainfall seasons, repeated doses may be required to fully cover the transmission period and achieve greater efficacy.

Anticipated single doses of fluralaner (750 mg) and afoxolaner (450 mg) in humans are comparable or lower than equivalent doses considered to be no-adverse effect levels (NOAEL) based on acute and repeat-dose toxicity studies in rats and dogs. Both fluralaner and afoxolaner were negative in genotoxicity studies and embryo-fetal development in rats was similarly unaffected. The veterinary application of these drugs provides therefore a first assessment of their safety. Interestingly, veterinary formulations of afoxolaner and fluralaner are based on racemic mixtures, whereas the S-enantiomer has been shown herein to be the active component against ectoparasites. Therefore profiling of the active enantiomer may indicate a 50%-reduction in the required dose.

### Example 13. Activity of Isoxazoline Compounds Against L. longipalpis, P. argentipes, and B. pahangi

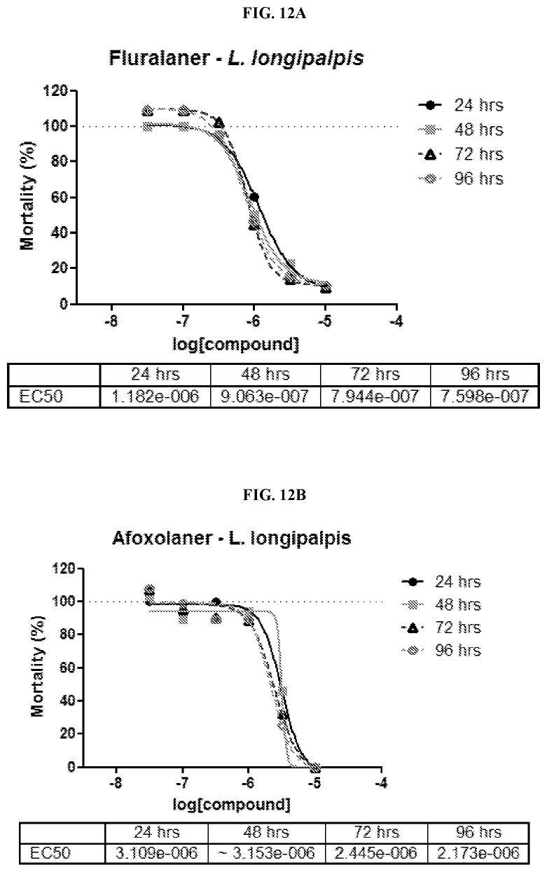

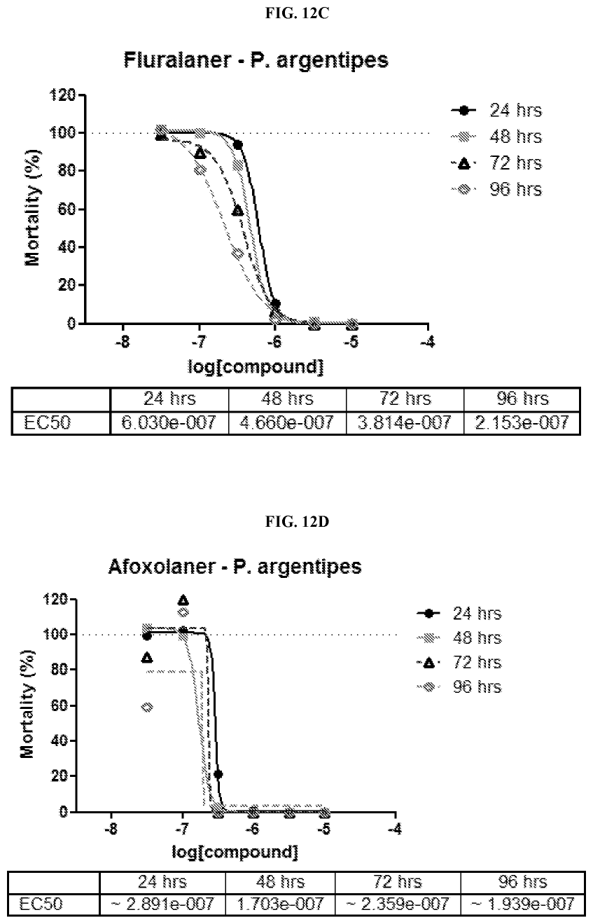

Isoxazoline compounds fluralaner or afoxolaner were reconstituted in 50% human serum and 50% red blood cells and administered to sand flies Lutzomyia longipalpis and Phlebotomus argentipes via standard membrane feeding. These san fly species are vectors for visceral leishmaniasis. FIG. 12A represents a plot of viable Lutzomyia longipalpis versus fluralaner concentration at various times of assessment. FIG. 12B represents a plot of viable Lutzomyia longipalpis versus afoxolaner concentration at various times of assessment. FIG. 12C represents a plot of viable Phlebotomus argentipes versus fluralaner concentration at various times of assessment. FIG. 12D represents a plot of viable Phlebotomus argentipes versus afoxolaner concentration at various times of assessment. The figures show that the isoxazoline compounds were effective at killing the sand flies in a dose-dependent manner. The EC50 values of each compound for sand fly death were determined by logistic regression using a least squares method to find the best fit and are shown in the figures.

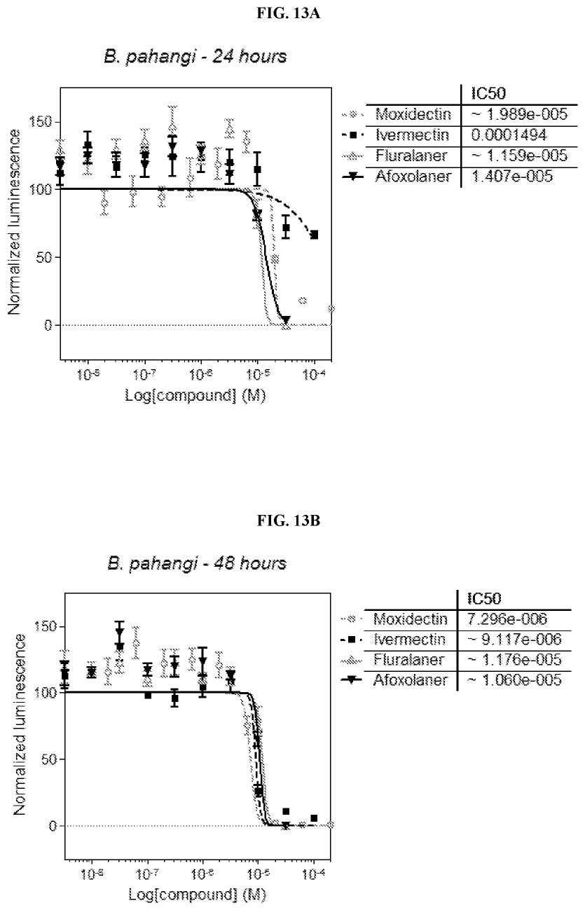

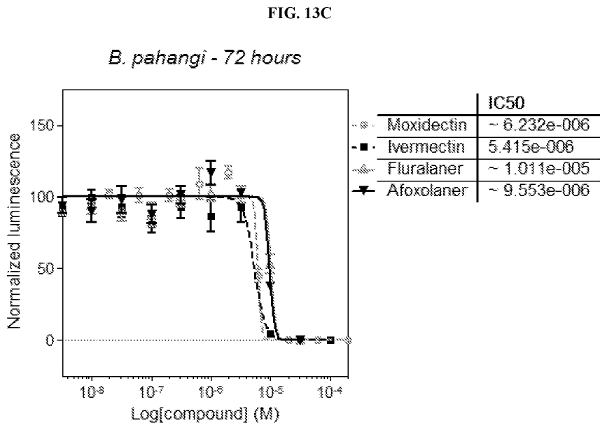

Fluralaner and afozolaner were also effective in killing Brugia pahangi, a nematode causing filariasis in cats and considered a model organism for human filariasis and other nematode diseases such as Onchocerciasis. Microfilariae of Brugia pahangi were were kept in 75 cm² flasks in RPMI-1640 with fetal bovine serum as described previously (Plos Negl. Trop. Dis. 2012, e1494). To test viability, 384-well plates were filled with 250 microfilariae per well. Serial dilutions of isoxazolines, ivermectin, moxidectin and equivalent vehicle concentration (0.5% DMSO) were added to the wells. Seventy two hours after treatment, viability was assessed with the Cell-Titer Glo reagent (Promega). FIG. 13A represents a plot of viable Brugia pahangi versus fluralaner, afoxolaner, or control compound (moxidectin and ivermectin) concentration as assessed at 24 hours. FIG. 13B represents a plot of viable Brugia pahangi versus fluralaner, afoxolaner, or control compound (moxidectin and ivermectin) concentration as assessed at 48 hours. FIG. 13C represents a plot of viable Brugia pahangi versus fluralaner, afoxolaner, or control compound (moxidectin and ivermectin) concentration as assessed at 72 hours. IC50 values are shown in the figures. As the isoxazolines are as active as moxidectin and ivermectin, these results suggest that the isoxazolines may be used for treatment of worm infections.

### Further Embodiments

1. A method of vector control comprising administering an insecticide to a human; wherein the insecticide is lethal to a vector exposed to the administered insecticide during a bite or blood meal with the human.
2. The method of embodiment 1, wherein the insecticide is lethal to the vector within 8, 7, 6, 5, 4, 3, 2 or 1 days of exposure.
3. The method of embodiment 1 or embodiment 2, wherein the insecticide is an ectoparasiticide.
4. The method of any of embodiments 1-3, wherein the insecticide is an isoxazoline compound.
5. The method of embodiment 4, wherein the isoxazoline compound has Formula (I), or pharmaceutically acceptable salt or solvate thereof:

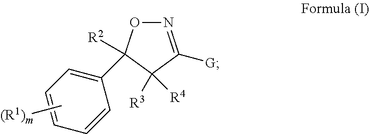

wherein:
each R¹ is independently selected from -D, —OR⁵, —SR⁵, —N(R⁶)(R⁷), —F, —Cl, —Br, —I, —C(O)R⁵, —CO₂R⁵, —CN, —NO₂, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
   each R⁵ is independently selected from —H, substituted or unsubstituted C₁-C₆alkyl, substituted or unsubstituted C₃-C₇cycloalkyl, substituted or unsubstituted aryl, and substituted or unsubstituted heteroaryl;
   each R⁶ and R⁷ are independently selected from —H, substituted or unsubstituted C₁-C₇alkyl, substituted or unsubstituted C₁-C₇fluoroalkyl, and substituted or unsubstituted C₁-C₇heteroalkyl;
   R⁶ and R⁷ can optionally be taken together with the N-atom to which they are attached to form a N-containing heterocycle;
R² is —H, —F, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, substituted or unsubstituted benzyl, or substituted or unsubstituted heteroaryl;
each R³ and R⁴ are independently selected from —H, —F, substituted or unsubstituted C₁-C₇alkyl, substituted or unsubstituted C₁-C₇fluoroalkyl, and substituted or unsubstituted C₁-C₇heteroalkyl; substituted or unsubstituted C₃-C₇cycloalkyl, substituted or unsubstituted C₂-C₆heterocycloalkyl, substituted or unsubstituted aryl, and substituted or unsubstituted heteroaryl;
m is 0, 1, 2, 3, 4, or 5; and
G is substituted or unsubstituted aryl or substituted or unsubstituted heteroaryl.
6. The method of embodiment 5, wherein G is

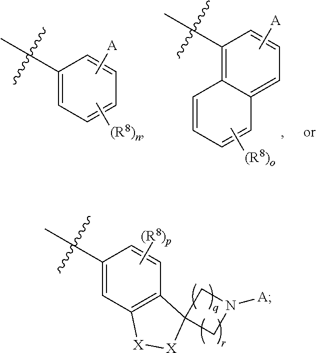

each R⁸ is independently selected from -D, —OR⁵, —SR⁵, —N(R⁶)(R⁷), —F, —Cl, —Br, —I, substituted or unsubstituted C₁-C₇alkyl, substituted or unsubstituted C₂-C₇alkenyl, substituted or unsubstituted C₂-C₇alkynyl, substituted or unsubstituted C₁-C₇fluoroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
two R⁸ groups can optionally be taken together with the adjacent carbon atoms to which they are attached to form aromatic or partially saturated carbocycle or heterocycle;
each X is independently selected from —O—, —S—, —S(═O)—, —S(═O)₂—, —NR⁶—, —C(═O)—, and —(CR⁹R¹⁰)ₛ—, wherein each R⁹ and R¹⁰ are independently selected from —H, -D, —F, —OR⁵, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl; s is 1, 2, or 3;
n is 0, 1, 2, 3, or 4;
o is 0, 1, 2, 3, 4, 5, or 6;
p is 0, 1, 2, or 3;
q is 0, 1, or 2;
r is 0, 1, or 2;
A is

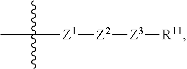

wherein
Z¹, Z², and Z³ are independently absent or selected from —(CR¹²R¹³)ᵤ—, —NR⁶—, —C(═O)—, —S(═O)—, —S(═O)₂—, —C(═O)(CR¹²R¹³)ᵤ—, —(CR¹²R¹³)ᵤC(═O)—, —C(═O)NR⁶—, —NR⁶C(═O)—, —C(═O)O—, —OC(═O)—, —OC(═O)NR⁶—, —NR⁶C(═O)O—, —NR⁶C(═O)NR⁶—, —C(═O)NR⁶(CR¹²R¹³)ᵤ—, —NR⁶C(═O)(CR¹²R¹³)ᵤ—, —(CR¹²R¹³)C(═O)NR⁶—, and —(CR¹²R¹³)ᵤNR⁶C(═O)—;
   each R¹² and R¹³ are independently selected from —H, -D, —F, —OR⁵, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl;
   u is 1, 2, 3, or 4; and
R¹¹ is substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, or substituted or unsubstituted heteroaryl.
7. The method of embodiment 6, wherein

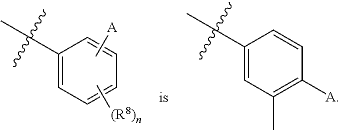

8. The method of embodiment 6, wherein

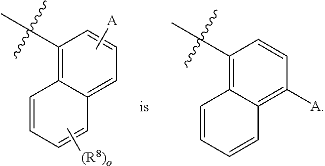

9. The method of embodiment 6, wherein

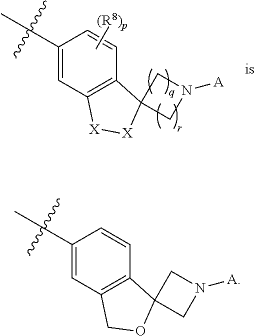

10. The method of any of embodiments 6-9, wherein A is

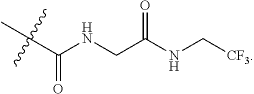 [SMILES: *C(=O)NCC(=O)NCC(F)(F)F]

11. The method of any of embodiments 6-9, wherein A is

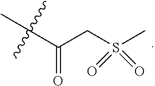 [SMILES: *C(=O)CS(C)(=O)=O]

12. The method of embodiment 10, wherein the compound of Formula (I) is fluralaner,

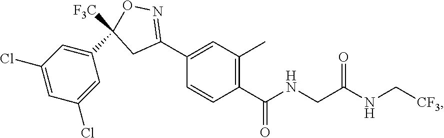 [SMILES: Cc1cc(C2=NO[C@](c3cc(Cl)cc(Cl)c3)(C(F)(F)F)C2)ccc1C(=O)NCC(=O)NCC(F)(F)F]

or pharmaceutically acceptable salt or solvate thereof.
13. The method of embodiment 10, wherein the compound of Formula (I) is afoxolaner,

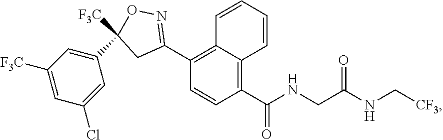 [SMILES: O=C(CNC(=O)c1ccc(C2=NO[C@](c3cc(Cl)cc(C(F)(F)F)c3)(C(F)(F)F)C2)c2ccccc12)NCC(F)(F)F]

or pharmaceutically acceptable salt or solvate thereof.
14. The method of embodiment 10, wherein the compound of Formula (I) is (S)-afoxolaner,

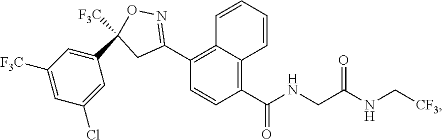 [SMILES: O=C(CNC(=O)c1ccc(C2=NO[C@@](c3cc(Cl)cc(C(F)(F)F)c3)(C(F)(F)F)C2)c2ccccc12)NCC(F)(F)F]

or pharmaceutically acceptable salt or solvate thereof.
15. The method of embodiment 10, wherein the compound of Formula (I) is (R)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide,

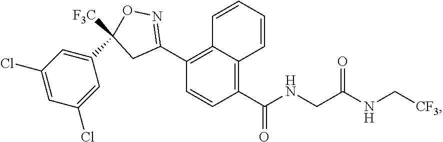 [SMILES: O=C(CNC(=O)c1ccc(C2=NO[C@](c3cc(Cl)cc(Cl)c3)(C(F)(F)F)C2)c2ccccc12)NCC(F)(F)F]

or pharmaceutically acceptable salt or solvate thereof.
16. The method of embodiment 10, wherein the compound of Formula (I) is (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide,

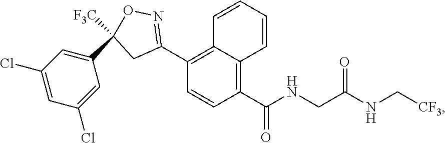 [SMILES: O=C(CNC(=O)c1ccc(C2=NO[C@@](c3cc(Cl)cc(Cl)c3)(C(F)(F)F)C2)c2ccccc12)NCC(F)(F)F]

or pharmaceutically acceptable salt or solvate thereof.
17. The method of embodiment 11, wherein the compound of Formula (I) is sarolaner,

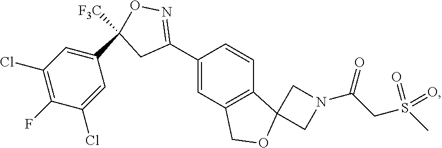 [SMILES: CS(=O)(=O)CC(=O)N1CC2(C1)OCc1cc(C3=NO[C@@](c4cc(Cl)c(F)c(Cl)c4)(C(F)(F)F)C3)ccc12]

or pharmaceutically acceptable salt or solvate thereof.
18. The method of embodiment 1, wherein the insecticide comprises fluralaner, afoxolaner, sarolaner, allethrin, resmethrin, phenothrin, etofenprox, permethrin, imidacloprid, fipronil, methoprene, fenoxycarb, pyriproxyfen, lufenuron, diflubenzuron, amitraz, selamectin, nitenpyram, dinotefuran, spinosad, or a pharmaceutically acceptable salt or derivative thereof.
19. The method of any of embodiments 1-18, wherein the insecticide targets the glutamate gated chloride channel
20. The method of any of embodiments 1-18, wherein the insecticide targets γ-aminobutyric acid (GABA)-gated chloride channel (GABAC1).
21. The method of embodiment 20, wherein the insecticide targets the γ-aminobutyric acid (GABA)-gated chloride channel in a location distinct from dieldrin.
22. The method of any of embodiments 19-21, wherein the vector has a mutation in the rdl locus conferring resistance to a cyclodiene, lindane, picrotoxinin, other convulsant, or a combination thereof.
23. The method of any of embodiments 19-21, wherein the vector has a mutation in the rdl locus conferring partial resistance to fipronil.
24. The method of embodiment 22, wherein the cyclodiene is dieldrin.
25. The method of embodiment 22, wherein the other convulsant comprises BIDN (3,3-bis(trifluoromethyl)bicyclo[2,2,1]heptane-2,2-dicarbonitrile), EBOB (ethynylbicycloorthobenzoate), or a combination thereof.
26. The method of any of embodiments 1-25, wherein the vector is an insect vector.
27. The method of embodiment 26, wherein the insect vector is selected from a mosquito, triatomine bug, tsetse fly, and black fly.
28. The method of embodiment 27, wherein the insect vector is a mosquito of a genus selected from Aedes, Anopheles, Culex, and Phlebotomus.
29. The method of embodiment 27 or embodiment 28, wherein the insect vector is a mosquito capable of transmitting a virus selected from a flavivirus, bunyavirus and a togavirus.
30. The method of embodiment 29, wherein the flavivirus is selected from zika virus, Japanese encephalitis, dengue virus, yellow fever virus, Powassan virus and usutu virus.
31. The method of embodiment 29, wherein the bunyavirus is selected from Rift Valley fever, Punta Toro virus, La Crosse virus, Maporal virus, Heartland virus, and Severe Fever thrombocytopenia syndrome virus.
32. The method of embodiment 29, wherein the togavirus is selected from Venezuelan equine encephalitis virus, Eastern equine encephalitis virus, Western equine encephalitis virus, and chikungunya virus.
33. The method of embodiment 28, wherein the insect vector is the Anopheles mosquito and the Anopheles mosquito is capable of transmitting o′nyong-nyong virus.
34. The method of embodiment 28, wherein the insect vector is the Anopheles mosquito and the Anopheles mosquito is capable of transmitting a Plasmodium parasite.
35. The method of embodiment 34, wherein the Plasmodium parasite is selected from P. falciparum, P. malariae, P. ovale, P. vivax and P. knowlesi.
36. The method of embodiment 34 or embodiment 35, wherein the Plasmodium parasite causes malaria.
37. The method of embodiment 28, wherein the insect vector is the Culex mosquito and the Culex mosquito is capable of transmitting a virus selected from Japanese encephalitis virus and West Nile virus.
38. The method of embodiment 28, wherein the insect vector is the Culex mosquito and the Culex mosquito is capable of transmitting a parasitic nematode.
39. The method of embodiment 38, wherein the parasitic nematode is Wuchereria bancrofti and/or a Brugia parasitic nematode.
40. The method of embodiment 28, wherein the insect vector is the Phlebotomus sandfly and the Phlebotomus sandfly is capable of transmitting a Leishmania parasite.
41. The method of embodiment 28, wherein the insect vector is the Phlebotomus sandfly and the Phlebotomus sandfly is capable of transmitting a virus within the Phlebovirus genus of the Bunyaviridae family.
42. The method of embodiment 27, wherein the insect vector is the triatomine bug and the triatomine bug is capable of transmitting a Trypanosoma cruzi parasite.
43. The method of embodiment 27, wherein the insect vector is the tsetse fly and the tsetse fly is capable of transmitting a Trypanosoma brucei parasite.
44. The method of embodiment 27, wherein the insect vector is the black fly and the black fly is capable of transmitting an Onchocerca volvulus parasite.
45. The method of any of embodiments 1-25, wherein the vector is an ectoparasite.
46. The method of embodiment 45, wherein the ectoparasite is selected from a tick and a flea.
47. The method of embodiment 45 or embodiment 46, wherein the ectoparasite is the tick and the tick is capable of transmitting a virus selected from Crimean-Congo haemorrhagic fever (CCHF) virus and tick-borne encephalitis virus.
48. The method of embodiment 45 or embodiment 46, wherein the ectoparasite is the tick and the tick is capable of transmitting a bacterium selected from Borrelia burgdorferi, Borrelia spirochetes, Anaplasma phagocytophilum, Ehrlichia chaffeensis, Ehrlichia muris, Ehrlichia ewingii, Neoehrlichia mikurensis, Rickettsia aeschlimannii, Rickettsia africae, Rickettsia australis, Rickettsia conorii, Rickettsia heilong-jiangensis, Rickettsia helvetica, Rickettsia honei, Rickettsia japonica, Rickettsia massiliae, Rickettsia monacensis, Rickettsia parkeri, Rickettsia raoultii, Rickettsia rickettsii, Rickettsia sibirica, Rickettsia sibiricamongolotimonae, Rickettsia slovaca, and Francisella tularensis.
49. The method of embodiment 45 or embodiment 46, wherein the ectoparasite is the flea and the flea is capable of transmitting a bacterium selected from Yersinia pestis, Rickettsia felis, and Rickettsia typhi.
50. The method of any of embodiments 1-49, wherein the insecticide is administered in an oral dosage form.
51. The method of any of embodiments 1-50, wherein a dose of between 1 mg/kg and 50 mg/kg of the insecticide is administered to the human.
52. The method of any of embodiments 1-51, wherein the insecticide is administered in a single dose, and the single dose is optionally repeated no more than every 3 months.
53. The method of embodiment 52, wherein the single dose is repeated no more than every 9-12 months.
54. The method of embodiment 52, wherein the single dose is administered once yearly for a period of 1, 2, 3, 4, 5, 6, 7, 8, 9 or 10 years.
55. The method of any of embodiments 52-54, wherein the single dose is effective in killing the exposed vector at least about 30, 40, 50, 60, 70, 80, 90, 100, 110, 120, 130, 140, 150, 160, 170, 180, 240, or 360 days after administration.
56. The method of any of embodiments 1-50, wherein the insecticide is administered in a single regimen comprising administration of a plurality of doses over a period of 1 week or less.
57. The method of embodiment 56, wherein the total dose of the insecticide administered in the single regimen is between 1 mg/kg and 50 mg/kg.
58. The method of embodiment 56 or 57, wherein the plurality of doses is 2, 3, 4 or 5 doses.
59. The method of any of embodiments 56-58, wherein the insecticide is administered over a period of 2, 3, 4, 5, 6 or 7 days.
60. The method of any of embodiments 56-59, wherein the single regimen is effective in killing the exposed vector at least about 30, 40, 50, 60, 70, 80, 90, 100, 110, 120, 130, 140, 150, 160, 170, 180, 240, or 360 days after administration.
61. The method of any of embodiments 1-60, wherein the vector is capable of transmitting a vector-borne disease to the human during a transmission season.
62. The method of embodiment 61, wherein the insecticide is administered within 30, 20, 10, 5, 4, 3, 2 or 1 day of the beginning of the transmission season.
63. The method of embodiment 61 or embodiment 62, wherein the transmission season correlates to a season having collectively an average daily rain fall that is higher than the average daily rain fall for an entire year.
64. The method of embodiment 61 or embodiment 62, wherein the transmission season is dependent on rainfall patterns, temperature and/or humidity.
65. The method of any of embodiments 61-64, wherein a calendar year comprises 1 transmission season.
66. The method of any of embodiments 61-65, wherein a calendar year comprises 2 or more transmission seasons.
67. The method of any of embodiments 61-66, wherein the transmission season comprises at least 30, 45, 60, 75, 90 or 120 days.
68. The method of any of embodiments 61-66, wherein the transmission season is less than or equal to about 180, 150, 120, 90, 75 or 60 days.
69. The method of any of embodiment 1-68, wherein the human is administered the insecticide prior to travel or deployment to a region comprising the vector.
70. The method of any of embodiments 1-68, wherein the human resides, works or is deployed within a region comprising the vector.
71. The method of embodiment 69 or embodiment 70, wherein there is a risk of the human acquiring a vector-borne disease after the bite or blood meal.
72. The method of embodiment 71, wherein the risk is described in the 2016, 2018, or a current edition of CDC Health Information for International Travel book as a high or moderate risk.
73. The method of any of embodiments 69-72, wherein the human is deployed to the region.
74. The method of embodiment 73, wherein the human is a military member.
75. The method of embodiment 73, wherein the human is a civilian.
76. The method of any of embodiments 1-75, wherein the human is about 5 years or older in age.
77. The method of any of embodiments 1-76, wherein the human is about 18 years or older in age.
78. The method of any of embodiments 1-77, wherein the human is one of a plurality of individuals in a human population and the plurality of individuals is administered the insecticide within an administration period.
79. The method of embodiment 78, wherein the plurality of individuals excludes those from the human population having an existing condition, contraindication to the insecticide, pregnant women, children under the age of 5, or a combination thereof.
80. The method of embodiment 78 or embodiment 79, wherein the plurality of individuals comprises at least about 50%, 60%, 70%, 80%, 85%, 90% or 95% of the human population.
81. The method of any of embodiments 78-80, provided that the plurality of individuals comprises at least 50% of the human population, the number of clinically identified disease transmissions between the administration period and about 3 months following the administration period is less than about 50% of the number of clinically identified transmissions for the same 3 month time period in one or more of the previous 10 years.
82. The method of any of embodiments 78-80, provided that the plurality of individuals comprises at least 50% of the human population, the number of vectors identified between the administration period and about 3 months following the administration period is less than about 50% of the number of vectors identified for the same 3 month time period in one or more of the previous 10 years.
83. The method of any of embodiments 1-82, further comprising administering to the human dihydroartemisinin-piperaquine; artemether and lumefantrine; artesunate and amodiaquine; artesunate and mefloquine; artesunate and sulfadoxine-pyrimethamine; primaquine; quinine and clindamycin; chloroquine; atovoquone/proguanil; or a combination thereof.
84. The method of any of embodiments 1-83, further comprising administering to the human ivermectin; albendazole; diethylcarbamazine citrate; ribavirin; pentavalent antimonials; ampthotericin B deoxycholate; paromycin; pentamidine isethionate; miltefosine; azoles medicines; pentamidine; suramin; melarsoprol; elfornithine; nifurtimox; antibiotic; or a combination thereof.
85. The method of embodiment 84, wherein the antibiotic is doxycycline.
86. The method of any of embodiments 1-85, wherein the human uses a bed-net to avoid the bite or blood meal with the vector.
87. The method of embodiment 86, wherein the bed-net comprises or is applied with a pyrethroid.
88. The method of any of embodiments 1-87, further comprising applying to a region in which the human resides, works or is deployed with an additional insecticide.
89. The method of embodiment 88, wherein the additional insecticide is a pyrethroid, an organochlorine, an organophosphate, a carbamate, a phenylpyrazole, a pyrrole, a macrocylcic lactone or a meta-diamide.
90. A method of preventing transmission of a disease-causing organism from a vector to a human population, the method comprising administering to each of a plurality of individuals of the population an insecticide in a single dose or single regimen over the course of less than or equal to 7 days; wherein the insecticide is present in one or more of the plurality of individuals at a concentration that is lethal to the vector within 30 days of the vector biting or engaging in a blood meal with the one or more of the plurality of individuals.
91. The method of embodiment 90, wherein the insecticide is present in the one or more of the plurality of individuals at a concentration that is lethal to the vector within 60 days of the vector biting or engaging in a blood meal with the one or more of the plurality of individuals.
92. The method of embodiment 90 or embodiment 91, wherein the insecticide is present in the one or more of the plurality of individuals at a concentration that is lethal to the vector within 90 days of the vector biting or engaging in a blood meal with the one or more of the plurality of individuals.
93. The method of any of embodiments 90-92, wherein the human population resides, works, travels, and/or is deployed within a region comprising the vector.
94. The method of embodiment 93, wherein at least one of the plurality of individuals is administered the insecticide prior to travel or deployment to the region.
95. The method of any of embodiments 90-94, wherein the plurality of individuals excludes those from the human population having an existing condition that is adverse to the insecticide.
96. The method of any of embodiments 90-95, wherein the plurality of individuals excludes those from the human population that are pregnant.
97. The method of any of embodiments 90-96, wherein the plurality of individuals excludes those from the human population that are nursing.
98. The method of any of embodiments 90-97, wherein the plurality of individuals excludes those from the human population that are children under the age of about 5.
99. The method of any of embodiments 90-98, wherein the plurality of individuals comprises at least about 50%, 60%, 70%, 80%, 85%, 90% or 95% of the human population.
100. The method of any of embodiments 90-99, wherein the plurality of individuals is administered the insecticide within an administration period.
101. The method of embodiment 100, wherein the administration period is between about 1 day and about 1 month.
102. The method of embodiment 100 or embodiment 101, wherein the number of clinically identified disease transmissions within the population that occur between the administration period and about 3 months following the administration period is less than about 50% of the number of clinically identified disease transmissions for the same 3 month time period in one or more of the previous 10 years.
103. The method of any of embodiments 100 or embodiment 101, wherein the number of vectors identified between the administration period and about 3 months following the administration period is less than about 50% of the number of vectors identified for the same 3 month time period in one or more of the previous 10 years.
104. The method of any of embodiments 90-103, wherein the single dose or single regimen comprises oral administration.
105. The method of any of embodiments 90-104, wherein between 1 mg/kg and 50 mg/kg of the insecticide is administered to the human in the single dose or single regimen.
106. The method of any of embodiments 90-105, wherein the insecticide is administered in the single dose, and the single dose is optionally repeated no more than every 3 months.
107. The method of embodiment 106, wherein the single dose is repeated no more than every 9-12 months.
108. The method of embodiment 106, wherein the single dose is administered once yearly for a period of 1, 2, 3, 4, 5, 6, 7, 8, 9 or 10 years.
109. The method of any of embodiments 90-105, wherein the insecticide is administered in the single regimen comprising a plurality of doses.
110. The method of embodiment 109, wherein the total dose of the insecticide administered in the single regimen is between 1 mg/kg and 50 mg/kg.
111. The method of embodiment 109 or embodiment 110, wherein the plurality of doses is 2, 3, 4 or 5 doses.
112. The method of any of embodiments 109-111, wherein the insecticide is administered over a period of 2, 3, 4, 5, 6 or 7 days.
113. The method of any of embodiments 90-112, wherein the vector is capable of transmitting the disease-causing organism to the human population during a transmission season.
114. The method of embodiment 113, wherein the insecticide is administered within 30, 20, 10, 5, 4, 3, 2 or 1 day of the beginning of the transmission season.
115. The method of embodiment 113 or embodiment 114, wherein the transmission season correlates to a season having collectively an average daily rain fall that is higher than the average daily rain fall for an entire year.
116. The method of embodiment 113 or embodiment 114, wherein the transmission season is dependent on rainfall patterns, temperature and/or humidity.
117. The method of any of embodiments 113-116, wherein a calendar year comprises 1 transmission season.
118. The method of any of embodiments 113-117, wherein a calendar year comprises 2 or more transmission seasons.
119. The method of any of embodiments 113-118, wherein the transmission season comprises at least 30, 45, 60, 75, 90, or 120 days.
120. The method of any of embodiments 113-118, wherein the transmission season is less than or equal to about 180, 150, 120, 90, 75 or 60 days.
121. The method of any of embodiments 90-120, wherein the insecticide is an ectoparasiticide.
122. The method of any of embodiments 90-121, wherein the insecticide is an isoxazoline compound.
123. The method of embodiment 122, wherein the isoxazoline compound has Formula (I), or pharmaceutically acceptable salt or solvate thereof:

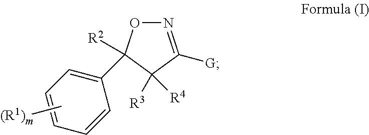

wherein:
each R¹ is independently selected from -D, —OR⁵, —SR⁵, —N(R⁶)(R⁷), —F, —Cl, —Br, —I, —C(O)R⁵, —CO₂R⁵, —CN, —NO₂, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
   each R⁵ is independently selected from —H, substituted or unsubstituted C₁-C₆alkyl, substituted or unsubstituted C₃-C₇cycloalkyl, substituted or unsubstituted aryl, and substituted or unsubstituted heteroaryl;
   each R⁶ and R⁷ are independently selected from —H, substituted or unsubstituted C₁-C₇alkyl, substituted or unsubstituted C₁-C₇fluoroalkyl, and substituted or unsubstituted C₁-C₇heteroalkyl;
   R⁶ and R⁷ can optionally be taken together with the N-atom to which they are attached to form a N-containing heterocycle;
R² is —H, —F, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, substituted or unsubstituted benzyl, or substituted or unsubstituted heteroaryl;
each R³ and R⁴ are independently selected from —H, —F, substituted or unsubstituted C₁-C₇alkyl, substituted or unsubstituted C₁-C₇fluoroalkyl, and substituted or unsubstituted C₁-C₇heteroalkyl; substituted or unsubstituted C₃-C₇cycloalkyl, substituted or unsubstituted C₂-C₆heterocycloalkyl, substituted or unsubstituted aryl, and substituted or unsubstituted heteroaryl;
m is 0, 1, 2, 3, 4, or 5; and
G is substituted or unsubstituted aryl or substituted or unsubstituted heteroaryl.
124. The method of embodiment 123, wherein G is

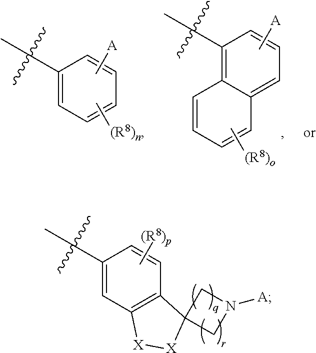

each R⁸ is independently selected from -D, —OR⁵, —SR⁵, —N(R⁶)(R⁷), —F, —Cl, —Br, —I, substituted or unsubstituted C₁-C₇alkyl, substituted or unsubstituted C₂-C₇alkenyl, substituted or unsubstituted C₂-C₇alkynyl, substituted or unsubstituted C₁-C₇fluoroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
two R⁸ groups can optionally be taken together with the adjacent carbon atoms to which they are attached to form aromatic or partially saturated carbocycle or heterocycle;
each X is independently selected from —O—, —S—, —S(═O)—, —S(═O)₂—, —C(═O)—, and —(CR⁹R¹⁰)ₛ—, wherein each R⁹ and R¹⁰ are independently selected from H, D, —F, —OR⁵, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl; s is 1, 2, or 3;
n is 0, 1, 2, 3, or 4;
o is 0, 1, 2, 3, 4, 5, or 6;
p is 0, 1, 2, or 3;
q is 0, 1, or 2;
r is 0, 1, or 2;
A is

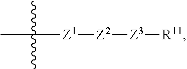

wherein
Z¹, Z², and Z³ are independently absent or selected from —(CR¹²R¹³)ᵤ—, —NR⁶—, —C(═O)—, —S(═O)—, —S(═O)₂—, —C(═O)(CR¹²R¹³)ᵤ—, —(CR¹²R¹³)ᵤC(═O)—, —C(═O)NR⁶—, —NR⁶C(═O)—, —C(═O)O—, —OC(═O)—, —OC(═O)NR⁶—, —NR⁶C(═O)O—, —NR⁶C(═O)NR⁶—, —C(═O)NR⁶(CR¹²R¹³)ᵤ—, —NR⁶C(═O)(CR¹²R¹³)ᵤ—, —(CR¹²R¹³)C(═O)NR⁶—, and —(CR¹²R¹³)ᵤNR⁶C(═O)—;
   each R¹² and R¹³ are independently selected from —H, -D, —F, —OR⁵, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl;
   u is 1, 2, 3, or 4; and
R¹¹ is substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, or substituted or unsubstituted heteroaryl.
125. The method of embodiment 124, wherein

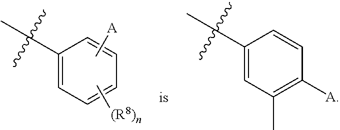

126. The method of embodiment 124, wherein

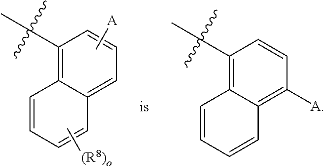

127. The method of embodiment 124, wherein

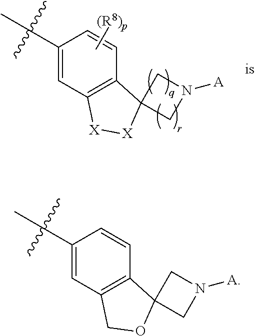

128. The method of any of embodiments 124-127, wherein A is

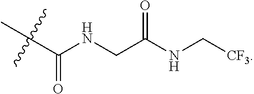 [SMILES: *C(=O)NCC(=O)NCC(F)(F)F]

129. The method of any of embodiments 124-127, wherein A is

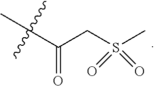 [SMILES: *C(=O)CS(C)(=O)=O]

130. The method of embodiment 128, wherein the compound of Formula (I) is fluralaner,

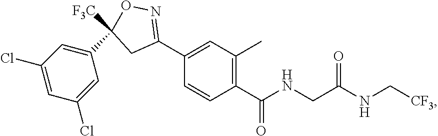 [SMILES: Cc1cc(C2=NO[C@](c3cc(Cl)cc(Cl)c3)(C(F)(F)F)C2)ccc1C(=O)NCC(=O)NCC(F)(F)F]

or pharmaceutically acceptable salt or solvate thereof.
131. The method of embodiment 128, wherein the compound of Formula (I) is afoxolaner,

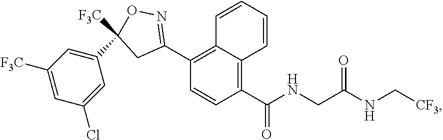 [SMILES: O=C(CNC(=O)c1ccc(C2=NO[C@](c3cc(Cl)cc(C(F)(F)F)c3)(C(F)(F)F)C2)c2ccccc12)NCC(F)(F)F]

or pharmaceutically acceptable salt or solvate thereof.
132. The method of embodiment 128, wherein the compound of Formula (I) is (S)-afoxolaner,

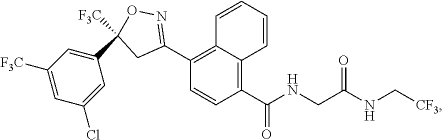 [SMILES: O=C(CNC(=O)c1ccc(C2=NO[C@@](c3cc(Cl)cc(C(F)(F)F)c3)(C(F)(F)F)C2)c2ccccc12)NCC(F)(F)F]

or pharmaceutically acceptable salt or solvate thereof.
133. The method of embodiment 128, wherein the compound of Formula (I) is (R)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide,

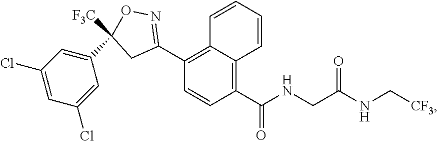 [SMILES: O=C(CNC(=O)c1ccc(C2=NO[C@](c3cc(Cl)cc(Cl)c3)(C(F)(F)F)C2)c2ccccc12)NCC(F)(F)F]

or pharmaceutically acceptable salt or solvate thereof.
134. The method of embodiment 128, wherein the compound of Formula (I) is (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide,

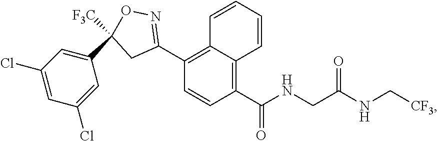 [SMILES: O=C(CNC(=O)c1ccc(C2=NO[C@@](c3cc(Cl)cc(Cl)c3)(C(F)(F)F)C2)c2ccccc12)NCC(F)(F)F]

or pharmaceutically acceptable salt or solvate thereof.
135. The method of embodiment 129, wherein the compound of Formula (I) is sarolaner,

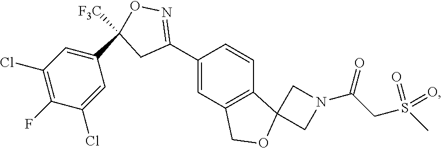 [SMILES: CS(=O)(=O)CC(=O)N1CC2(C1)OCc1cc(C3=NO[C@@](c4cc(Cl)c(F)c(Cl)c4)(C(F)(F)F)C3)ccc12]

or pharmaceutically acceptable salt or solvate thereof.
136. The method of any of embodiments 90-120, wherein the insecticide comprises fluralaner, afoxolaner, sarolaner, allethrin, resmethrin, phenothrin, etofenprox, permethrin, imidacloprid, fipronil, methoprene, fenoxycarb, pyriproxyfen, lufenuron, diflubenzuron, amitraz, selamectin, nitenpyram, dinotefuran, spinosad, or a pharmaceutically acceptable salt or derivative thereof.
137. The method of any of embodiments 90-121, wherein the insecticide targets the glutamate gated chloride channel
138. The method of any of embodiments 90-121, wherein the insecticide targets γ-aminobutyric acid (GABA)-gated chloride channel (GABAC1).
139. The method of embodiment 138, wherein the insecticide targets the γ-aminobutyric acid (GABA)-gated chloride channel in a location distinct from dieldrin.
140. The method of any of embodiments 137-139, wherein the vector has a mutation in the rdl locus conferring resistance to a cyclodiene, lindane, picrotoxinin, other convulsant, or a combination thereof.
141. The method of any of embodiments 137-140, wherein the vector has a mutation in the rdl locus conferring partial resistance to fipronil.
142. The method of embodiment 140, wherein the cyclodiene is dieldrin.
143. The method of embodiment 140, wherein the other convulsant comprises BIDN, EBOB, or a combination thereof.
144. The method of any of embodiments 90-143, wherein the vector is an insect vector.
145. The method of embodiment 144, wherein the insect vector is selected from a mosquito, triatomine bug, tsetse fly, and black fly.
146. The method of embodiment 145, wherein the insect vector is a mosquito of a genus selected from Aedes, Anopheles, Culex, and Phlebotomus.
147. The method of embodiment 145 or embodiment 146, wherein the insect vector is a mosquito capable of transmitting a virus selected from a flavivirus, bunyavirus and a togavirus.
148. The method of embodiment 147, wherein the flavivirus is selected from zika virus, Japanese encephalitis, dengue virus, yellow fever virus, Powassan virus and usutu virus.
149. The method of embodiment 147, wherein the bunyavirus is selected from Rift Valley fever, Punta Toro virus, La Crosse virus, Maporal virus, Heartland virus, and Severe Fever thrombocytopenia syndrome virus.
150. The method of embodiment 147, wherein the togavirus is selected from Venezuelan equine encephalitis virus, Eastern equine encephalitis virus, Western equine encephalitis virus, and chikungunya virus.
151. The method of embodiment 146, wherein the insect vector is the Anopheles mosquito and the Anopheles mosquito is capable of transmitting o′nyong-nyong virus.
152. The method of embodiment 146, wherein the insect vector is the Anopheles mosquito and the Anopheles mosquito is capable of transmitting a Plasmodium parasite.
153. The method of embodiment 152, wherein the Plasmodium parasite is selected from P. falciparum, P. malariae, P. ovale, P. vivax and P. knowlesi.
154. The method of embodiment 152 or embodiment 153, wherein the Plasmodium parasite causes malaria.
155. The method of embodiment 146, wherein the insect vector is the Culex mosquito and the Culex mosquito is capable of transmitting a virus selected from Japanese encephalitis virus and West Nile virus.
156. The method of embodiment 146, wherein the insect vector is the Culex mosquito and the Culex mosquito is capable of transmitting a parasitic nematode.
157. The method of embodiment 156, wherein the parasitic nematode is Wuchereria bancrofti and/or a Brugia parasitic nematode.
158. The method of embodiment 146, wherein the insect vector is the Phlebotomus sandfly and the Phlebotomus sandfly is capable of transmitting a Leishmania parasite.
159. The method of embodiment 146, wherein the insect vector is the Phlebotomus sandfly and the Phlebotomus sandfly is capable of transmitting a virus within the Phlebovirus genus of the Bunyaviridae family.
160. The method of embodiment 145, wherein the insect vector is the triatomine bug and the triatomine bug is capable of transmitting a Trypanosoma cruzi parasite.
161. The method of embodiment 145, wherein the insect vector is the tsetse fly and the tsetse fly is capable of transmitting a Trypanosoma brucei parasite.
162. The method of embodiment 145, wherein the insect vector is the black fly and the black fly is capable of transmitting an Onchocerca volvulus parasite.
163. The method of any of embodiments 90-143, wherein the vector is an ectoparasite.
164. The method of embodiment 163, wherein the ectoparasite is selected from a tick and a flea.
165. The method of embodiment 163 and embodiment 164, wherein the ectoparasite is the tick and the tick is capable of transmitting a virus selected from Crimean-Congo haemorrhagic fever (CCHF) virus and tick-borne encephalitis virus.
166. The method of embodiment 163 and embodiment 164, wherein the ectoparasite is the tick and the tick is capable of transmitting a bacterium selected from Borrelia burgdorferi, Borrelia spirochetes, Anaplasma phagocytophilum, Ehrlichia chaffeensis, Ehrlichia muris, Ehrlichia ewingii, Neoehrlichia mikurensis, Rickettsia aeschlimannii, Rickettsia africae, Rickettsia australis, Rickettsia conorii, Rickettsia heilong-jiangensis, Rickettsia helvetica, Rickettsia honei, Rickettsia japonica, Rickettsia massiliae, Rickettsia monacensis, Rickettsia parkeri, Rickettsia raoultii, Rickettsia rickettsii, Rickettsia sibirica, Rickettsia sibiricamongolotimonae, Rickettsia slovaca, and Francisella tularensis.
167. The method of embodiment 163 and embodiment 164, wherein the ectoparasite is the flea and the flea is capable of transmitting a bacterium selected from Yersinia pestis, Rickettsia felis, and Rickettsia typhi.
168. The method of any of embodiments 90-167, further comprising administering to the plurality of individuals dihydroartemisinin-piperaquine; artemether and lumefantrine; artesunate and amodiaquine; artesunate and mefloquine; artesunate and sulfadoxine-pyrimethamine; primaquine; quinine and clindamycin; chloroquine; atovoquone/proguanil; or a combination thereof.
169. The method of any of embodiments 90-168, further comprising administering to the human ivermectin; albendazole; diethylcarbamazine citrate; ribavirin; pentavalent antimonials; ampthotericin B deoxycholate; paromycin; pentamidine isethionate; miltefosine; azoles medicines; pentamidine; suramin; melarsoprol; elfornithine; nifurtimox; antibiotic; or a combination thereof.
170. The method of embodiment 169, wherein the antibiotic comprises doxycycline.
171. The method of any of embodiments 90-170, wherein one or more members of the human population uses a bed-net to avoid the bite or blood meal with the vector.
172. The method of embodiment 171, wherein the bed-net comprises or is applied with a pyrethroid.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the invention. Furthermore, all examples and conditional language recited herein are principally intended to aid the reader in understanding the principles of this disclosure and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. It is intended that the following claims define the scope of the disclosure and that methods and structures within the scope of these claims and their equivalents be covered thereby.

What is claimed is:

1. A method of vector control comprising administering an insecticide to a human; wherein the insecticide is lethal to a vector exposed to the administered insecticide during a bite or blood meal with the human.

2. The method of claim 1, wherein the human is administered the insecticide in: (a) a single dose or (b) a plurality of doses over a course of less than or equal to about 3 days; and wherein the single dose or the plurality of doses is administered once or not more frequently than every 3 months.

3. The method of claim 2, wherein the single dose or the plurality of doses is administered not more frequently than every 9 months.

4. The method of any of claims 1-3, wherein if the vector is exposed to the administered insecticide within about 30, 60, 90, or 120 days after administration, the administered insecticide is effective in killing the vector.

5. The method of any of claims 1-4, wherein the insecticide is lethal to the vector within about 8, 7, 6, 5, 4, 3, 2 or 1 days of exposure.

6. The method of any of claims 1-5, wherein the vector is an insect vector selected from a mosquito, triatomine bug, tsetse fly, sandfly, and black fly.

7. The method of claim 6, wherein the insect vector is a mosquito of a genus selected from Aedes, Anopheles, Culex, and Phlebotomus.

8. The method of claim 6 or claim 7, wherein the insect vector is a mosquito capable of transmitting a parasite.

9. The method of claim 8, wherein the parasite is of the Plasmodium genus.

10. The method of claim 7 or claim 8, wherein the insect vector is a mosquito capable of transmitting a virus selected from a flavivirus, bunyavirus, and a togavirus.

11. The method of any of claims 1-10, wherein the insecticide is an ectoparasiticide.

12. The method of any of claims 1-11, wherein the insecticide is an isoxazoline compound.

13. The method of any of claims 1-12, wherein the insecticide is a compound having Formula (I), or pharmaceutically acceptable salt or solvate thereof: 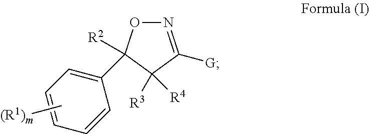
wherein:
each R¹ is independently selected from -D, —OR⁵, —SR⁵, —N(R⁶)(R⁷), —F, —Cl, —Br, —I, —C(O)R⁵, —CO₂R⁵, —CN, —NO₂, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
each R⁵ is independently selected from —H, substituted or unsubstituted C₁-C₆alkyl, substituted or unsubstituted C₃-C₇cycloalkyl, substituted or unsubstituted aryl, and substituted or unsubstituted heteroaryl;
each R⁶ and R⁷ are independently selected from —H, substituted or unsubstituted C₁-C₇alkyl, substituted or unsubstituted C₁-C₇fluoroalkyl, and substituted or unsubstituted C₁-C₇heteroalkyl;
R⁶ and R⁷ can optionally be taken together with the N-atom to which they are attached to form a N-containing heterocycle;
R² is —H, —F, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, substituted or unsubstituted benzyl, or substituted or unsubstituted heteroaryl;
each R³ and R⁴ are independently selected from —H, —F, substituted or unsubstituted C₁-C₇alkyl, substituted or unsubstituted C₁-C₇fluoroalkyl, and substituted or unsubstituted C₁-C₇heteroalkyl; substituted or unsubstituted C₃-C₇cycloalkyl, substituted or unsubstituted C₂-C₆heterocycloalkyl, substituted or unsubstituted aryl, and substituted or unsubstituted heteroaryl;
m is 0, 1, 2, 3, 4, or 5; and
G is substituted or unsubstituted aryl or substituted or unsubstituted heteroaryl.

14. The method of claim 13, wherein G is 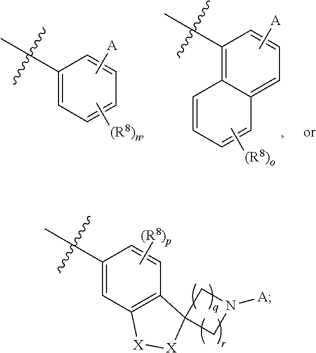
each R⁸ is independently selected from -D, —OR⁵, —SR⁵, —N(R⁶)(R⁷), —F, —Cl, —Br, —I, substituted or unsubstituted C₁-C₇alkyl, substituted or unsubstituted C₂-C₇alkenyl, substituted or unsubstituted C₂-C₇alkynyl, substituted or unsubstituted C₁-C₇fluoroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
two R⁸ groups can optionally be taken together with the adjacent carbon atoms to which they are attached to form aromatic or partially saturated carbocycle or heterocycle;
each X is independently selected from —O—, —S—, —S(═O)—, —S(═O)₂—, —NR⁶—, —C(═O)—, and —(CR⁹R¹⁰)ₛ—, wherein each R⁹ and R¹⁰ are independently selected from —H, -D, —F, —OR⁵, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl; s is 1, 2, or 3;
n is 0, 1, 2, 3, or 4;
o is 0, 1, 2, 3, 4, 5, or 6;
p is 0, 1, 2, or 3;
q is 0, 1, or 2;
r is 0, 1, or 2;
A is 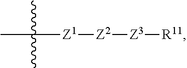
wherein
Z¹, Z², and Z³ are independently absent or selected from —(CR¹²R¹³)ᵤ—, —NR⁶—, —C(═O)—, —S(═O)—, —S(═O)₂—, —C(═O)(CR¹²R¹³)ᵤ—, —(CR¹²R¹³)ᵤC(═O)—, —C(═O)NR⁶—, —NR⁶C(═O)—, —C(═O)O—, —OC(═O)—, —OC(═O)NR⁶—, —NR⁶C(═O)O—, —NR⁶C(═O)NR⁶—, —C(═O)NR⁶(CR¹²R¹³)ᵤ—, —NR⁶C(═O)(CR¹²R¹³)ᵤ—, —(CR¹²R¹³)ᵤC(═O)NR⁶—, and —(CR¹²R¹³)ᵤNR⁶C(═O)—;
each R¹² and R¹³ are independently selected from —H, -D, —F, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl;
u is 1, 2, 3, or 4; and
R¹¹ is substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, or substituted or unsubstituted heteroaryl.

15. The method of claim 14, wherein

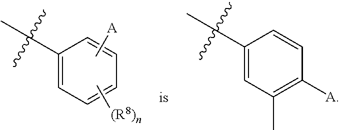

16. The method of claim 14, wherein

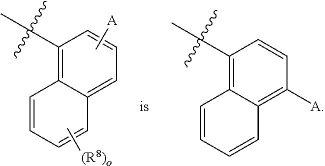

17. The method of claim 14, wherein

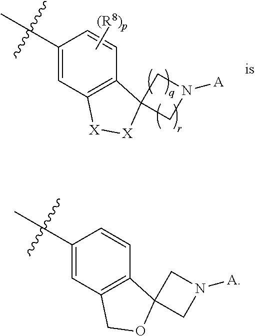

18. The method of any of claims 14-17, wherein A is

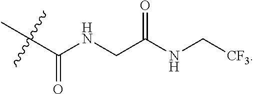 [SMILES: *C(=O)NCC(=O)NCC(F)(F)F]

19. The method of any of claims 14-17, wherein A is

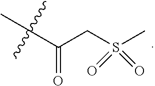 [SMILES: *C(=O)CS(C)(=O)=O]

20. The method of claim 18, wherein the compound of Formula (I) is fluralaner, 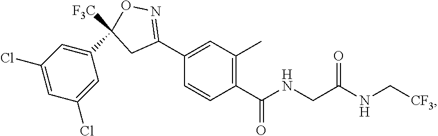 [SMILES: Cc1cc(C2=NO[C@](c3cc(Cl)cc(Cl)c3)(C(F)(F)F)C2)ccc1C(=O)NCC(=O)NCC(F)(F)F]
or pharmaceutically acceptable salt or solvate thereof.

21. The method of claim 18, wherein the compound of Formula (I) is (S)-fluralaner, 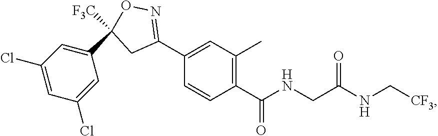 [SMILES: Cc1cc(C2=NO[C@@](c3cc(Cl)cc(Cl)c3)(C(F)(F)F)C2)ccc1C(=O)NCC(=O)NCC(F)(F)F]
or pharmaceutically acceptable salt or solvate thereof.

22. The method of claim 18, wherein the compound of Formula (I) is afoxolaner, 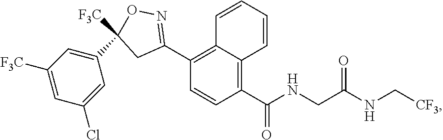 [SMILES: O=C(CNC(=O)c1ccc(C2=NO[C@](c3cc(Cl)cc(C(F)(F)F)c3)(C(F)(F)F)C2)c2ccccc12)NCC(F)(F)F]
or pharmaceutically acceptable salt or solvate thereof.

23. The method of claim 18, wherein the compound of Formula (I) is (S)-afoxolaner, 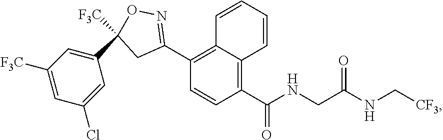 [SMILES: O=C(CNC(=O)c1ccc(C2=NO[C@@](c3cc(Cl)cc(C(F)(F)F)c3)(C(F)(F)F)C2)c2ccccc12)NCC(F)(F)F]
or pharmaceutically acceptable salt or solvate thereof.

24. The method of claim 18, wherein the compound of Formula (I) is (R)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide, 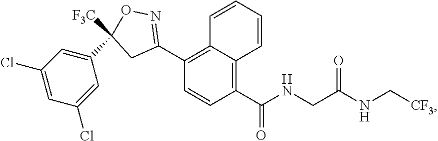 [SMILES: O=C(CNC(=O)c1ccc(C2=NO[C@](c3cc(Cl)cc(Cl)c3)(C(F)(F)F)C2)c2ccccc12)NCC(F)(F)F]
or pharmaceutically acceptable salt or solvate thereof.

25. The method of claim 18, wherein the compound of Formula (I) is (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide, 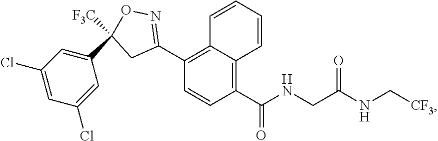 [SMILES: O=C(CNC(=O)c1ccc(C2=NO[C@@](c3cc(Cl)cc(Cl)c3)(C(F)(F)F)C2)c2ccccc12)NCC(F)(F)F]
or pharmaceutically acceptable salt or solvate thereof.

26. The method of claim 19, wherein the compound of Formula (I) is sarolaner, 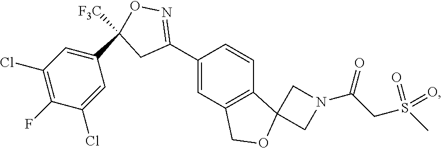 [SMILES: CS(=O)(=O)CC(=O)N1CC2(C1)OCc1cc(C3=NO[C@@](c4cc(Cl)c(F)c(Cl)c4)(C(F)(F)F)C3)ccc12]
or pharmaceutically acceptable salt or solvate thereof.

27. The method of any of claims 1-26, wherein the insecticide is administered in an oral dosage form.

28. The method of any of claims 1-27, wherein each dose of the insecticide administered to the human is between about 1 mg/kg and about 50 mg/kg.

29. The method of any of claims 1-27, wherein each dose of the insecticide administered to the human is between about 150 mg and about 750 mg.

30. A method of preventing transmission of a disease-causing organism from a vector to a human population, the method comprising administering to each of a plurality of individuals of the population an insecticide; wherein the vector is exposed to the administered insecticide during a bite or blood meal with a member of the plurality of individuals, and if the vector is exposed to the administered insecticide within about 30, 60, 90, or 120 days after administration, the administered insecticide is effective in killing the vector.

31. The method of claim 30, wherein the insecticide is administered to each of the plurality of individuals in a single dose, and the single dose is optionally repeated no more than every 3 months.

32. The method of claim 30, wherein the insecticide is administered to each of the plurality of individuals in a plurality of doses over a course of less than or equal to about 3 days, and the plurality of doses is optionally repeated no more than every 3 months.

33. The method of any of claims 30-32, wherein the vector is an insect vector selected from a mosquito, triatomine bug, tsetse fly, sandfly, and black fly.

34. The method of claim 33, wherein the insect vector is a mosquito of a genus selected from Aedes, Anopheles, Culex, and Phlebotomus.

35. The method of claim 33 or claim 34, wherein the insect vector is a mosquito capable of transmitting a parasite.

36. The method of claim 35, wherein the parasite is of the Plasmodium genus.

37. The method of claim 33 or claim 34, wherein the insect vector is a mosquito capable of transmitting a virus selected from a flavivirus, bunyavirus, and a togavirus.

38. The method of any of claims 30-37, wherein the insecticide is administered in an oral dosage form.

39. The method of any of claims 30-38, wherein each dose of the insecticide administered to the plurality of individuals is between about 1 mg/kg and about 50 mg/kg.

40. The method of any of claims 30-38, wherein each dose of the insecticide administered to the plurality of individuals is between about 150 mg and about 750 mg.

41. The method of any of claims 30-40, wherein the insecticide is an ectoparasiticide.

42. The method of any of claims 30-41, wherein the insecticide is an isoxazoline compound.

43. The method of any of claims 30-42, wherein the insecticide is a compound having Formula (I), or pharmaceutically acceptable salt or solvate thereof: 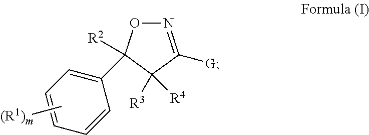
wherein:
each R¹ is independently selected from -D, —OR⁵, —SR⁵, —N(R⁶)(R⁷), —F, —Cl, —Br, —I, —C(O)R⁵, —CO₂R⁵, —CN, —NO₂, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
each R⁵ is independently selected from —H, substituted or unsubstituted C₁-C₆alkyl, substituted or unsubstituted C₃-C₇cycloalkyl, substituted or unsubstituted aryl, and substituted or unsubstituted heteroaryl;
each R⁶ and R⁷ are independently selected from —H, substituted or unsubstituted C₁-C₇alkyl, substituted or unsubstituted C₁-C₇fluoroalkyl, and substituted or unsubstituted C₁-C₇heteroalkyl;
R⁶ and R⁷ can optionally be taken together with the N-atom to which they are attached to form a N-containing heterocycle;
R² is —H, —F, substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, substituted or unsubstituted benzyl, or substituted or unsubstituted heteroaryl;
each R³ and R⁴ are independently selected from —H, —F, substituted or unsubstituted C₁-C₇alkyl, substituted or unsubstituted C₁-C₇fluoroalkyl, and substituted or unsubstituted C₁-C₇heteroalkyl; substituted or unsubstituted C₃-C₇cycloalkyl, substituted or unsubstituted C₂-C₆heterocycloalkyl, substituted or unsubstituted aryl, and substituted or unsubstituted heteroaryl;
m is 0, 1, 2, 3, 4, or 5; and
G is substituted or unsubstituted aryl or substituted or unsubstituted heteroaryl.

44. The method of claim 43, wherein G is 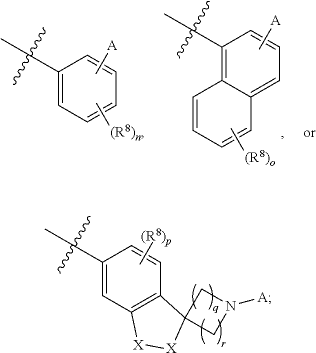
each R⁸ is independently selected from -D, —OR⁵, —SR⁵, —N(R⁶)(R⁷), —F, —Cl, —Br, —I, substituted or unsubstituted C₁-C₇alkyl, substituted or unsubstituted C₂-C₇alkenyl, substituted or unsubstituted C₂-C₇alkynyl, substituted or unsubstituted C₁-C₇fluoroalkyl, substituted or unsubstituted C₃-C₇cycloalkyl, substituted or unsubstituted C₂-C₆heterocycloalkyl, substituted or unsubstituted aryl, and substituted or unsubstituted heteroaryl;
two R⁸ groups can optionally be taken together with the adjacent carbon atoms to which they are attached to form aromatic or partially saturated carbocycle or heterocycle;
each X is independently selected from —O—, —S—, —S(═O)—, —S(═O)₂—, —NR⁶—, —C(═O)—, and —(CR⁹R¹⁰)ₛ—, wherein each R⁹ and R¹⁰ are independently selected from —H, -D, —F, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl; s is 1, 2, or 3;
n is 0, 1, 2, 3, or 4;
o is 0, 1, 2, 3, 4, 5, or 6;
p is 0, 1, 2, or 3;
q is 0, 1, or 2;
r is 0, 1, or 2;
A is 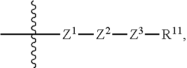
wherein
Z¹, Z², and Z³ are independently absent or selected from —(CR¹²R¹³)ᵤ—, —NR⁶—, —C(═O)—, —S(═O)—, —S(═O)₂—, —C(═O)(CR¹²R¹³)ᵤ—, —(CR¹²R¹³)ᵤC(═O)—, —C(═O)NR⁶—, —NR⁶C(═O)—, —C(═O)O—, —OC(═O)—, —OC(═O)NR⁶—, —NR⁶C(═O)O—, —NR⁶C(═O)NR⁶—, —C(═O)NR⁶(CR¹²R¹³)ᵤ—, —NR⁶C(═O)(CR¹²R¹³)ᵤ—, —(CR¹²R¹³)ᵤC(═O)NR⁶—, and —(CR¹²R¹³)ᵤNR⁶C(═O)—;
each R¹² and R¹³ are independently selected from —H, -D, —F, —OR⁵, —C(O)R⁵, substituted or unsubstituted C₁-C₇alkyl; substituted or unsubstituted C₃-C₇cycloalkyl, substituted or unsubstituted C₂-C₇heterocycloalkyl, substituted or unsubstituted aryl, and substituted or unsubstituted heteroaryl;
u is 1, 2, 3, or 4; and
R¹¹ is substituted or unsubstituted C₁-C₇alkyl, substituted or unsubstituted C₁-C₇fluoroalkyl, substituted or unsubstituted C₁-C₇heteroalkyl, substituted or unsubstituted C₃-C₇cycloalkyl, substituted or unsubstituted C₂-C₆heterocycloalkyl, substituted or unsubstituted aryl, or substituted or unsubstituted heteroaryl.

45. The method of claim 44, wherein

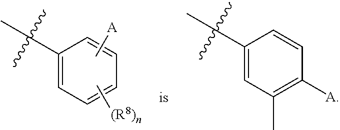

46. The method of claim 44, wherein

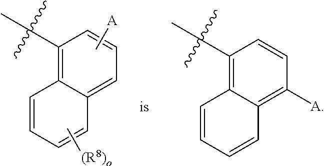

47. The method of claim 44, wherein

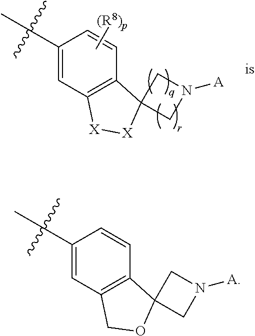

48. The method of any of claims 44-47, wherein A is

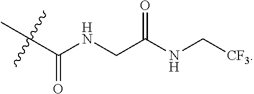 [SMILES: *C(=O)NCC(=O)NCC(F)(F)F]

49. The method of any of claims 44-47, wherein A is

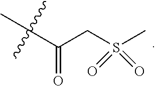 [SMILES: *C(=O)CS(C)(=O)=O]

50. The method of claim 48, wherein the compound of Formula (I) is fluralaner, 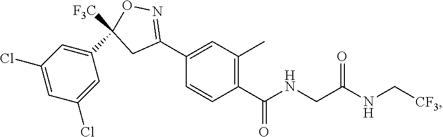 [SMILES: Cc1cc(C2=NO[C@](c3cc(Cl)cc(Cl)c3)(C(F)(F)F)C2)ccc1C(=O)NCC(=O)NCC(F)(F)F]
or pharmaceutically acceptable salt or solvate thereof.

51. The method of claim 48, wherein the compound of Formula (I) is (S)-fluralaner, 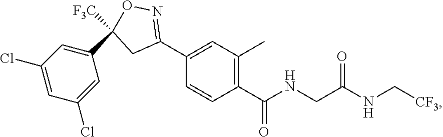 [SMILES: Cc1cc(C2=NO[C@@](c3cc(Cl)cc(Cl)c3)(C(F)(F)F)C2)ccc1C(=O)NCC(=O)NCC(F)(F)F]
or pharmaceutically acceptable salt or solvate thereof.

52. The method of claim 48, wherein the compound of Formula (I) is afoxolaner, 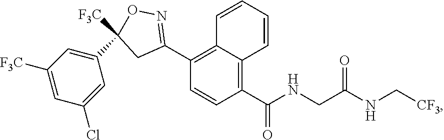 [SMILES: O=C(CNC(=O)c1ccc(C2=NO[C@](c3cc(Cl)cc(C(F)(F)F)c3)(C(F)(F)F)C2)c2ccccc12)NCC(F)(F)F]
or pharmaceutically acceptable salt or solvate thereof.

53. The method of claim 48, wherein the compound of Formula (I) is (S)-afoxolaner, 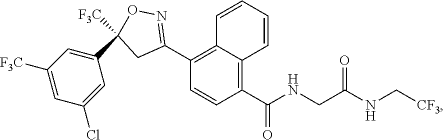 [SMILES: O=C(CNC(=O)c1ccc(C2=NO[C@@](c3cc(Cl)cc(C(F)(F)F)c3)(C(F)(F)F)C2)c2ccccc12)NCC(F)(F)F]
or pharmaceutically acceptable salt or solvate thereof.

54. The method of claim 48, wherein the compound of Formula (I) is (R)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide, 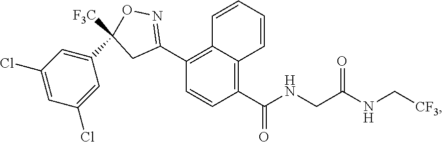 [SMILES: O=C(CNC(=O)c1ccc(C2=NO[C@](c3cc(Cl)cc(Cl)c3)(C(F)(F)F)C2)c2ccccc12)NCC(F)(F)F]
or pharmaceutically acceptable salt or solvate thereof.

55. The method of claim 48, wherein the compound of Formula (I) is (S)-4-(5-(3,5-dichlorophenyl)-5-(trifluoromethyl)-4,5-dihydroisoxazol-3-yl)-N-(2-oxo-2-((2,2,2-trifluoroethyl)amino)ethyl)-1-naphthamide, 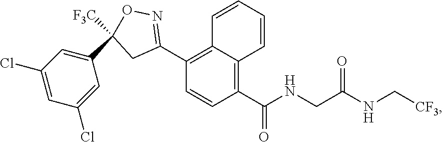 [SMILES: O=C(CNC(=O)c1ccc(C2=NO[C@@](c3cc(Cl)cc(Cl)c3)(C(F)(F)F)C2)c2ccccc12)NCC(F)(F)F]
or pharmaceutically acceptable salt or solvate thereof.

56. The method of claim 49, wherein the compound of Formula (I) is sarolaner, 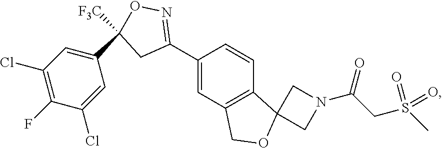 [SMILES: CS(=O)(=O)CC(=O)N1CC2(C1)OCc1cc(C3=NO[C@@](c4cc(Cl)c(F)c(Cl)c4)(C(F)(F)F)C3)ccc12]
or pharmaceutically acceptable salt or solvate thereof.
